(19) United States
(12) Patent Application Publication
(10) Pub. No.: US 2024/0164267 A1
(43) Pub. Date: May 23, 2024

(54) GENETIC MECHANISM TO ENHANCE HEALTHY STEROLS AND HARD ENDOSPERM IN SEEDS

(71) Applicant: Iowa State University Research Foundation, Inc., Ames, IA (US)

(72) Inventors: Walter P. SUZA, Ames, IA (US); Siddique Imran ABOOBUCKER, Ames, IA (US)

(21) Appl. No.: 18/501,470

(22) Filed: Nov. 3, 2023

Related U.S. Application Data
(60) Provisional application No. 63/382,252, filed on Nov. 3, 2022.

(51) Int. Cl. A01H 1/00; A01H 6/46

(52) U.S. Cl. CPC A01H 1/105; A01H 6/4684

(57) ABSTRACT

The disclosure relates to CYP710A genes associated with enhanced healthy sterols in plants. Also disclosed are plants comprising altered expression or activity of the CYP710A genes along with related methods for enhancing healthy sterols in plants. Also provided are oils and compositions containing altered sterol levels.

### CROSS REFERENCE TO RELATED APPLICATIONS

This application claims priority to provisional application U.S. Ser. No. 63/382,252, filed Nov. 3, 2022, which is incorporated herein by reference in its entirety.

### SEQUENCE LISTING XML

The instant application contains a sequence listing, which has been submitted in XML file format by electronic submission and is hereby incorporated by reference in its entirety. The XML file, created on Nov. 3, 2023, is named P14104US01.xml and is 7,641 bytes in size.

### TECHNICAL FIELD

The present disclosure relates to the field of biotechnology. More specifically, the present disclosure relates to compositions and methods for enhancing healthy sterols in plants, particularly in maize seed.

### BACKGROUND

Nutritional quality of grains and cereals is a complex trait. It is a key determinant of the market value as well as a major constraint in plant breeding. Quality is influenced by both genetic and environmental factors. There have been several attempts to genetically improve the nutritional quality of plants with varied success.

### SUMMARY

The present disclosure relates to modified plants and their progeny having improved nutritional characteristics. More particularly, the present disclosure relates to a novel approach to enhance healthy phytosterol and food grade quality maize seed. This disclosure further offers an advantage to increase healthy phytosterol in maize seed, thereby avoiding the need for external supplementation of phytosterols. In addition, the disclosure relates to a genetic mechanism to increase hard endosperm for food grade quality maize seed. Thus, the present disclosure provides compositions and methods for increasing healthy phytosterols (sitosterol) in plants, particularly in maize seeds, as well as increasing hard endosperm of seeds.

Modified plants with increased sitosterol or hard endosperm are provided, the plants comprise reduced expression or activity of an endogenous CYP710A gene relative to a corresponding unmodified plant. In certain embodiments, the modified plants comprise one or more nucleotide modifications introduced through a targeted DNA break at a genomic locus of a plant comprising the CYP710A gene. In certain embodiments, the modified plants comprise a silencing element that targets the endogenous CYP710A gene.

Progeny, plant parts, plant cells, seeds, and asexual propagates of the modified plants are also provided.

Methods for producing a plant with increased sitosterol or hard endosperm are provided, the methods comprise reducing expression or activity of an endogenous CYP710A gene in the plant.

In certain embodiments, the endogenous CYP710A gene encodes a polypeptide comprising an amino acid sequence having at least 80%, at least 90%, at least 95%, at least 99%, or 100% sequence identity to SEQ ID NO: 2. In certain embodiments, the endogenous CYP710A gene comprises a nucleotide sequence having at least 80%, at least 90%, at least 95%, or at least 99% sequence identity to SEQ ID NO: 1.

Expression constructs, vectors, biological samples, plants, plant parts, plant cells comprising the polynucleotides of the disclosure are also provided.

Commodity plant products prepared from the aforementioned plants, plant parts, and plant cells are provided. Methods for producing a commodity plant product comprising processing the aforementioned plants or plant parts to obtain the product are also provided.

While multiple embodiments are disclosed, still other embodiments of the present disclosure will become apparent based on the detailed description, which shows and describes illustrative embodiments of the disclosure. Accordingly, the figures and detailed description are to be regarded as illustrative in nature and not restrictive.

### BRIEF DESCRIPTION OF THE FIGURES

The following drawings form part of the disclosure and are included to further demonstrate certain embodiments. In some instances, embodiments can be best understood by referring to the accompanying figures in combination with the detailed description presented herein. The description and accompanying figures may highlight a certain specific example, or a certain embodiment. However, one skilled in the art will understand that portions of the example or embodiment may be used in combination with other examples or embodiments.

FIG. 1A-C shows stigmasterol biosynthesis and the phylogenetic relationship of CYP710A-like homologues. FIG. 1A shows the biosynthetic pathway of plant sterols leading to stigmasterol. Stigmasterol synthesis starts from acetate followed by a series of steps. Plants produce a mixture of sterols, in addition to stigmasterol, including campesterol (24-methyl) and sitosterol. The enzyme, sterol C-22 desaturase, catalyzes the conversion of sitosterol to stigmasterol. Campesterol is the preferred precursor of brassinosteroids (BR). Dashed arrows indicate multiple steps and solid arrows denote single step in the pathway. HMGS, 3-hydroxy-3-methylglutaryl-CoA synthase; HMGR, 3-hydroxy-3-methylglutaryl-CoA reductase; SQS, squalene synthase; SMT1, sterol methyltransferase 1; SMT2/3, sterol methyltransferase2/3; DWF1, C-24 sterol reductase; CYP710A, sterol C-22 desaturase. FIG. 1B is a cartoon representation of the mechanism of sterol C-22 desaturase mediated conversion of sitosterol to stigmasterol. The site of desaturation is marked in circles. FIG. 1C shows the phylogenetic relationship of the CYP710A-like homologues. Bootstrap values are shown as percentages (based on 1,000 replicates). Saccharomyces cerevisiae CYP61 was used to root the tree. Zm, Zea mays; Sb, Sorghum bicolor; Bradi, Brachypodium distachyon; Ta, Triticum aestivum; Hv, Hordeum vulgare; Os, Oryza sativa; Le, Lycopersicum esculentum; Sotub, Solanum tuberosum; Nt, Nicotiana tabacum; Medtr, Medicago truncatula; Phvul, Phaseolus vulgaris; Glyma, Glycine max; At, Arabidopsis thaliana; Sc, Saccharomyces cerevisiae.

FIG. 2A-C shows heterologous expression of ZmCYP710A8 in Arabidopsis increases stigmasterol at the expense of sitosterol. FIG. 2A is a schematic of the T-DNA region of the molecular construct used to overexpress the presumed coding region of ZmCYP710A8 sequence in Arabidopsis thaliana Col-0 background. LB, left border; RB, right border; 35S, cauliflower mosaic virus constitutive promoter; Bar, phosphinothricin N-acetyltransferase gene as selectable marker; VSP, vegetative storage protein terminator; NOS, nopaline synthase terminator. FIG. 2B shows free major sterol contents of transgenic Arabidopsis thaliana rosette leaves expressing the maize gene ZmCYP710A8 in Col-0 background. Also shown, in the bottom panel, is the quantification of ZmCYP710A8 mRNA in the rosette leaves relative to AtEF1α-A (used as reference gene for normalization). WT, Col-0 wild type; Empty, Col-0 transformed with empty vector; L4, L8, L11, L15 and L22 are five independent transgenic events. Tissue samples were pooled from rosette leaves of 10 individual plants (4-week-old) for each line, split to do both sterol and mRNA analysis. Sterol measurements are shown as means±SD from four technical replicates. mRNA measurements are shown as means±SD from three technical replicates. FIG. 2C shows images of 4-week-old wild type and a ZmCYP710A8 overexpressor (L22) Arabidopsis plants.

FIG. 3A-D shows expression of mRNAs for ZmCYP710A8 and sterol biosynthesis genes correlate with stigmasterol composition during maize development. FIG. 3A shows a representative B73 seedling at the V1 stage and the different organs used in the analysis. FIG. 3B shows the relative mRNA levels of different sterol biosynthesis genes. Tissue samples were pooled from 3 individual seedlings in one experiment. mRNA quantification was done in three replicates. ZmACTIN was used as reference gene for normalization. Data are means±SE from two independent experiments. FIG. 3C shows stigmasterol composition (% of total sterols) and quantification of ZmCYP710A8 mRNA in different maize genotypes. Tissue samples for mRNA and sterol analysis are as described in FIG. 3B. Root is as shown in FIG. 3A and shoot is a pool of all the above ground tissue shown in FIG. 3A. Data are means±SE from two independent experiments. FIG. 3D shows stigmasterol composition (% of total sterols) of the different organs of maize B73 at V1 stage. Tissue samples are as described in FIG. 3B. Sterols were measured in four technical replicates for each experiment. Data are means±SE from two independent experiments. One-way ANOVA was performed at a 95% confidence interval. Means that do not share a letter were significantly different.

FIG. 4 shows relative expression of ZmCYP710A8 mRNA in response to hormone treatments. Relative mRNA levels (log₂) of ZmCYP710A8 compared to mock in response to various hormone treatments for indicated hours after treatment. Leaf tissues (leaf 2) from three plants were pooled for one time point (as indicated) per treatment. mRNA levels were measured in triplicates with ZmACTIN as the reference gene for normalization. Data are means±SE from two independent experiments. Asterisks indicate statistical significance by Students t-test compared to mock samples at their respective time points. *P<0.05 ***P<0.001; ns, not significant.

FIG. 5A-F shows ZmCYP710A8 is the sole maize C-22 sterol desaturase to synthesize stigmasterol. FIG. 5A is a schematic of the ZmCYP710A8 gene structure with its 5′ and 3′ UTRs. The coding sequence is shown as a box with the start (ATG) and stop (TGA) codons marked. Inverted triangles show the site of transposable element insertions in Zmcyp710a8-1 and Zmcyp710a8-2 mutants. The primer binding sites used for genotyping the mutations are indicated with 103F, 200R and TIR6. FIG. 5B shows identification of transposon insertion in the Zmcyp710a8 mutants by PCR genotyping. The top panel shows PCR reactions amplified using one primer binding to the transposon and the other to the genome and the bottom panel shows amplicons from both primers binding to the genome. Transposon insertion would not lead to amplification of wild type band in mutants. Primer pairs are as shown in FIG. 5A. W22 is the wild type control. DNA ladder is also shown. FIG. 5C shows representative images of 11-day old wild type and mutant maize seedlings grown by cigar roll method. Scale bar=5 cm. FIG. 5D shows representative GC-MS total ion chromatogram traces of root tissues from Zmcyp710a8-1, Zmcyp710a8-2 mutants and wild type (W22). The three major sterols are indicated. FIG. 5E shows the composition (% of total sterols) of free major sterols in shoot and root of the two mutants and wild type. Tissue samples were pooled from 3 individual seedlings in one experiment. Sterol extraction was performed for two independent experiments and measured in four technical replicates for each experiment and the data shown are means±SD. Root is as shown in FIG. 5C and shoot is a pool of all the above ground tissue shown in FIG. 5C. FIG. 5F shows mass spectrometry images of sterols in roots of 3-day-old maize seedlings. Root tip end is on the left side of images. The monoisotopic mass for each sterol were monitored with both major isotopes of silver (¹⁰⁷Ag and ¹⁰⁹Ag). The MS images are of sitosterol silver adducts (m/z 523.2913 and 521.2913) and stigmasterol silver adducts (m/z 519.2856 and 517.2865) are presented. Respective bright field images are included as the special reference for the MSI data. MSI images were captured using a 50 μm raster size. The images are representative of two experiments. Scale bar=1 mm.

FIG. 6A-D shows the impact of exogenous stigmasterol on the expression of maize sterol biosynthesis genes. FIG. 6A-B shows relative mRNA quantification of ZmHMGR and ZmSMT2 genes in wild type (W22) in response to feeding 10 μM stigmasterol at different time points (FIG. 6A), and in response to 10 μM stigmasterol and 10 μM sitosterol feeding for 4 hours (FIG. 6B). FIG. 6C-D show relative mRNA quantification of ZmHMGR and ZmSMT2 genes in Zmcyp710a8-1 background in response to various sterols (10 μM) for 4 hours in roots (FIG. 6C) and in shoots (FIG. 6D). ZmACTIN served as the reference gene for normalization. Data are means±SD. Asterisks indicate statistical significance by Students t-test compared to 0 h in FIG. 6A and to Mock in FIG. 6B, FIG. 6C, and FIG. 6D. *P<0.05 **P<0.01 ***P<0.001; ns, not significant.

FIG. 7A-B shows stigmasterol modification impacts metabolite profile in root and shoot tissues of Zmcyp710a8 mutants. FIG. 7A shows non-targeted metabolite profile from root and shoot of wild type and the Zmcyp710a8 mutants. Relative metabolite levels in the two maize mutants compared to their wild type are shown as fold changes in Root and Shoot. FIG. 7B shows fold changes of selected metabolites in the root tissue of Zmcyp710a8-1 mutant with exogenous stigmasterol feeding. Asterisks indicate statistical significance by Students t-test compared to Zmcyp710a8-1. *P<0.05 **P<0.01 ***P<0.001.

FIG. 8A-C shows stigmasterol modification impacts metabolite profile in root and shoot tissues of Arabidopsis Atcyp710a1 expressing ZmCYP710A8. FIG. 8A shows relative quantification of ZmCYP710A8 mRNA in two independent Arabidopsis lines (L12 and L13) expressing the 35S:ZmCYP710A8 construct in Atcyp710a1 background. Root and shoot tissue samples were separately pooled from >100 seedlings (3-week-old) grown in magenta boxes. Data are means±SD of three replicates. AtEF1α-A was used as the internal control. ND, not detected. FIG. 8B shows the composition (% of total sterols) of stigmasterol in root and shoot of Col-0, Atcyp710a1 mutant and lines L12 and L13. Tissue samples were pooled from >100 seedlings (3-week-old) grown in magenta boxes. Roots and shoots were harvested separately from one box for a biological replicate. Data are means±SD from three biological replicates. FIG. 8C shows non-targeted metabolite profile from root and shoot of Col-0, Atcyp710a1 mutant and lines L12 and L13. Relative metabolite levels compared to wild type are shown as fold changes in root and shoot.

FIG. 9 shows healthy phytosterol (sitosterol) is increased by Zmcyp710a8 mutation in maize seeds. Free sterol content in seeds of the three indicated genotypes were measured using Gas Chromatography-Mass Spectrometry method. Total sterol content is increased in Zmcyp710a8 mutants compared to wild type (W22). Stigmasterol is absent in Zmcyp710a8-1 and is present only in trace amounts in Zmcyp710a8-2. Campesterol levels remain the same in all three genotypes. Data are means of three replicates.

FIG. 10A-D shows hard endosperm (improved food grade) of maize seeds is increased by Zmcyp710a8-1 mutation. FIG. 10A shows Zmcyp710a8-1 kernels display more hard endosperm than soft endosperm compared to W22 (wild type) kernels. FIG. 10B shows hard and soft endosperm in each kernel were quantified using ImageJ software. Percentage area of hard or soft endosperm relative to total kernel area is shown. Data are from 18 kernels for each genotype. FIG. 10C-D shows seed weight and density is reduced in Zmcyp710a8-1 kernels compared to wild type indicative of increased hard endosperm.

FIG. 10C shows seed weight in grams per 1,000 seeds is shown. Data are from two measurements. FIG. 10D shows density was calculated by Near Infrared Spectroscopy measurement using >500 kernels. Data are from two replicates.

### DETAILED DESCRIPTION

So that the present disclosure may be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments of the present disclosure without undue experimentation, the preferred materials and methods are described herein. In describing and claiming the embodiments of the present disclosure, the following terminology will be used in accordance with the definitions set out below.

It is to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Similarly, the word “or” is intended to include “and” unless the context clearly indicate otherwise. The word “or” means any one member of a particular list and also includes any combination of members of that list. Further, all units, prefixes, and symbols may be denoted in its SI accepted form.

Numeric ranges recited within the specification are inclusive of the numbers defining the range and include each integer within the defined range. Throughout this disclosure, various embodiments of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾. This applies regardless of the breadth of the range.

As used herein, the phrase “biological sample” refers to either intact or non-intact (e.g., milled seed or plant tissue, chopped plant tissue, lyophilized tissue) plant tissue. It may also be an extract comprising intact or non-intact seed or plant tissue. The biological sample can comprise flour, meal, flakes, syrup, oil, starch, and cereals manufactured in whole or in part to contain crop plant by-products. In certain embodiments, the biological sample is “non-regenerable” (i.e., incapable of being regenerated into a plant or plant part).

The term “cis-element” generally refers to transcriptional regulatory element that affects or modulates expression of an operably linked transcribable polynucleotide, where the transcribable polynucleotide is present in the same DNA sequence. A cis-element may function to bind transcription factors, which are trans-acting polypeptides that regulate transcription.

As used herein, the term “elite line” means any line that has resulted from breeding and selection for superior agronomic performance.

The term “endogenous” relates to any gene or nucleic acid sequence that is already present in a cell.

An “enhancer” element is any nucleic acid molecule that increases transcription of a nucleic acid molecule when functionally linked to a promoter regardless of its relative position.

By “essentially identical” or “essentially complementary” is meant that a polynucleotide (or at least one strand of a double-stranded polynucleotide) are designed to hybridize under physiological conditions in cells of a plant to an endogenous gene or to RNA transcribed from the endogenous gene to effect regulation or suppression of the endogenous gene.

The term “expression”, as used herein, generally refers to the production of a functional end-product e.g., an mRNA or a protein (precursor or mature).

As used herein, “gene” includes a nucleic acid fragment that expresses a functional molecule such as, but not limited to, a specific protein coding sequence and regulatory elements, such as those preceding (5′ non-coding sequences) and following (3′ non-coding sequences) the coding sequence.

A “genomic locus” as used herein generally refers to the location on a chromosome of the plant where a gene is found.

As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA).

An “intron” is an intervening sequence in a gene that is transcribed into RNA but is then excised in the process of generating the mature mRNA. The term is also used for the excised RNA sequences. An “exon” is a portion of the sequence of a gene that is transcribed and is found in the mature messenger RNA derived from the gene but is not necessarily a part of the sequence that encodes the final gene product.

The terms “knock-out”, “gene knock-out” and “genetic knock-out” are used interchangeably herein. A knock-out represents a DNA sequence of a cell that has been rendered partially or completely inoperative; such a DNA sequence prior to knock-out could have encoded an amino acid sequence, or could have had a regulatory function (e.g., promoter), for example. A knock-out may be produced by an indel (insertion or deletion of nucleotide bases in a target DNA sequence through NHEJ), or by specific removal of sequence that reduces or completely destroys the function of sequence at or near the targeting site.

As used herein, the term “maize” means Zea mays or corn and includes all plant varieties that can be bred with corn, including wild maize species.

As used herein, “modified”, in the context of plants, seeds, plant components, plant cells, and plant genomes, refers to a state containing changes or variations from their natural or native state. For instance, a “native transcript” of a gene refers to an RNA transcript that is generated from an unmodified gene. Typically, a native transcript is a sense transcript. Modified plants or seeds contain molecular changes in their genetic materials, including either genetic or epigenetic modifications. Typically, modified plants or seeds, or a parental or progenitor line thereof, have been subjected to mutagenesis, genome editing (e.g., without being limiting, via methods using site-specific nucleases), genetic transformation (e.g., without being limiting, via methods of Agrobacterium transformation or microprojectile bombardment), or a combination thereof. In one embodiment, a modified plant provided herein comprises no non-plant genetic material or sequences. In certain embodiments, a modified plant provided herein comprises no interspecies genetic material or sequences.

As used herein, the terms “nucleic acid,” “nucleic acid molecule,” “nucleotide sequence” and “polynucleotide” can be used interchangeably and encompass both RNA and DNA, including cDNA, genomic DNA, mRNA, synthetic (e.g., chemically synthesized) DNA or RNA and chimeras of RNA and DNA. The term polynucleotide, nucleotide sequence, or nucleic acid refers to a chain of nucleotides without regard to length of the chain. The nucleic acid can be double-stranded or single-stranded. Where single-stranded, the nucleic acid can be a sense strand or an antisense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases. The present disclosure further provides a nucleic acid that is the complement (which can be either a full complement or a partial complement) of a nucleic acid, nucleotide sequence, or polynucleotide.

As used herein, “plant” refers to a whole plant, any part thereof, or a cell or tissue culture derived from a plant, comprising any of: whole plants, plant components or organs (e.g., leaves, stems, roots, etc.), plant tissues, seeds, plant cells, and/or progeny of the same. A progeny plant can be from any filial generation, e.g., F1, F2, F3, F4, F5, F6, F7, etc. A plant cell is a biological cell of a plant, taken from a plant or derived through culture from a cell taken from a plant.

The terms “polypeptide,” “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

A “promoter” generally refers to a nucleic acid fragment capable of controlling transcription of another nucleic acid fragment. A promoter generally includes a core promoter (also known as minimal promoter) sequence that includes a minimal regulatory region to initiate transcription, that is a transcription start site. Generally, a core promoter includes a TATA box and a GC rich region associated with a CAAT box or a CCAAT box. These elements act to bind RNA polymerase II to the promoter and assist the polymerase in locating the RNA initiation site. Some promoters may not have a TATA box or CAAT box or a CCAAT box, but instead may contain an initiator element for the transcription initiation site. A core promoter is a minimal sequence required to direct transcription initiation and generally may not include enhancers or other UTRs.

A “regulatory element” generally refers to a transcriptional regulatory element involved in regulating the transcription of a nucleic acid molecule such as a gene or a target gene. The regulatory element is a nucleic acid and may include a promoter, an enhancer, an intron, a 5′-untranslated region (5′-UTR, also known as a leader sequence), or a 3′-UTR or a combination thereof. A regulatory element may act in “cis” or “trans”, and generally it acts in “cis”, i.e. it activates expression of genes located on the same nucleic acid molecule, e.g. a chromosome, where the regulatory element is located.

A “repressor” (also sometimes called herein silencer) is defined as any nucleic acid molecule which inhibits the transcription when functionally linked to a promoter regardless of relative position.

As used herein, the term “sterol” refers to unsaturated hydroxyl group-containing derivatives of a fused, reduced ring system, cyclopenta[c]-phenanthrene, comprising three fused cyclohexane rings (A, B and C) in a phenanthrene arrangement, and a terminal cyclopentane ring (D). The exemplary steroid below illustrates the numbering system employed herein in describing the location of groups and substituents.

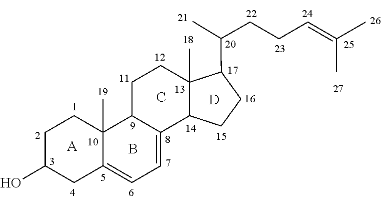

Sterols may or may not contain a C-5 to C-6 double bond, as this is a feature introduced late in the biosynthetic pathway. Sterols contain a C₈-C₁₀ side chain at the C-17 position, as shown above.

Sterol C-22 desaturase (EC 2.7.3.9) catalyzes the formation of a double bond at C-22 on the side chain. This formation of a double bond at C-22 on the side chain marks the end of the sterol compound biosynthetic pathway, and results in the formation of stigmasterol (Benveniste (1986) Annu. Rev. Plant Physiol. 37:275-308).

The term “phytosterol,” which applies to sterols found uniquely in plants, refers to a sterol containing a C-5, and in some cases a C-22, double bond. Phytosterols are further characterized by alkylation of the C-17 side-chain with a methyl or ethyl substituent at the C-24 position. Major phytosterols include, but are not limited to, sitosterol, stigmasterol, campesterol, brassicasterol, etc. Cholesterol, which lacks a C-24 methyl or ethyl side chain, is found in plants but is not unique thereto, and is not a “phytosterol”.

### Cytochrome P450 CYP710A Genes

CYP710A genes are provided that are associated with enhanced healthy sterols (sitosterol) and increased hard endosperm in plants. Such sequences include the amino acid sequence set forth in SEQ ID NO: 2. Also provided are polynucleotide sequences encoding such amino acid sequences, including SEQ ID NO: 1. In certain embodiments, the CYP710A gene encodes a polypeptide comprising an amino acid sequence having at least 80%, at least 85%, at least 90%, at least 91%, at least 92%, at least 93%, at least 94%, at least 95%, at least 96%, at least 97%, at least 98%, at least 99%, at least 99.5%, or at least 99.9% sequence identity to the full length or a fragment of the amino acid sequence of SEQ ID NO: 2. In certain embodiments, the CYP710A gene comprises a nucleotide sequence at least 80%, at least 85%, at least 90%, at least 91%, at least 92%, at least 93%, at least 94%, at least 95%, at least 96%, at least 97%, at least 98%, at least 99%, at least 99.5%, or at least 99.9% to the full length or a fragment of the nucleotide sequence of SEQ ID NO: 1.

Sequence alignments and percent identity calculations may be determined using a variety of comparison methods designed to detect similar or identical sequences including, but not limited to, the Megalign® program of the LASERGENE® bioinformatics computing suite (DNASTAR® Inc., Madison, Wis.). Unless stated otherwise, multiple alignment of the sequences provided herein were performed using the Clustal V method of alignment (Higgins and Sharp (1989) CAB/OS. 5:151-153) with the default parameters (GAP PENALTY=10, GAP LENGTH PENALTY=10). Default parameters for pairwise alignments and calculation of percent identity of protein sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner.

Alternatively, the Clustal W method of alignment may be used. The Clustal W method of alignment (described by Higgins and Sharp, CAB/OS. 5:151-153 (1989); Higgins, D. G. et al., Comput. Appl. Biosci. 8:189-191 (1992)) can be found in the MegAlign™ v6.1 program of the LASERGENE® bioinformatics computing suite (DNASTAR® Inc., Madison, Wis.). Default parameters for multiple alignment correspond to GAP PENALTY=10, GAP LENGTH PENALTY=0.2, Delay Divergent Sequences=30%, DNA Transition Weight=0.5, Protein Weight Matrix=Gonnet Series, DNA Weight Matrix=IUB. For pairwise alignments the default parameters are Alignment=Slow-Accurate, Gap Penalty=10.0, Gap Length=0.10, Protein Weight Matrix=Gonnet 250 and DNA Weight Matrix=IUB. After alignment of the sequences using the Clustal W program, it is possible to obtain “percent identity” and “divergence” values by viewing the “sequence distances” table in the same program. In one embodiment the % sequence identity is determined over the entire length of the molecule (nucleotide or amino acid).

Sequences that are homologous, i.e., that share significant sequence identity or similarity, to those provided herein are also part of the present disclosure. Homologous sequences can be derived from any plant including monocots and dicots and in particular agriculturally important plant species. In addition, homologous sequences may be derived from plants that are evolutionarily-related to crop plants, but which may not have yet been used as crop plants.

Homologous sequences can comprise orthologous or paralogous sequences. Several different methods are known by those of skill in the art for identifying and defining these functionally homologous sequences. Three general methods for defining orthologs and paralogs are described; an ortholog, paralog or homolog may be identified by one or more of the methods described below.

Orthologs and paralogs are evolutionarily related genes that have similar sequence and similar functions. Orthologs are structurally related genes in different species that are derived by a speciation event. Paralogs are structurally related genes within a single species that are derived by a duplication event.

Within a single plant species, gene duplication may result in two copies of a particular gene, giving rise to two or more genes with similar sequence and often similar function known as paralogs. A paralog is therefore a similar gene formed by duplication within the same species. Paralogs typically cluster together or in the same Glade (a group of similar genes) when a gene family phylogeny is analyzed using programs such as CLUSTAL (Thompson et al. (1994) Nucleic Acids Res. 22: 4673-4680; Higgins et al. (1996) Methods Enzymol. 266: 383-402). Groups of similar genes can also be identified with pair-wise BLAST analysis (Feng and Doolittle (1987) J. Mol. Evol. 25: 351-360). Analysis of groups of similar genes with similar function that fall within one Glade can yield sub-sequences that are particular to the Glade. These sub-sequences, known as consensus sequences, can not only be used to define the sequences within each Glade, but define the functions of these genes; genes within a Glade may contain paralogous sequences, or orthologous sequences that share the same function (see also, for example, Mount (2001), in Bioinformatics: Sequence and Genome Analysis Cold Spring Harbor Laboratory Press, Cold Spring Harbor, N.Y., page 543.)

Speciation, the production of new species from a parental species, can also give rise to two or more genes with similar sequence and similar function. These genes, termed orthologs, often have an identical function within their host plants and are often interchangeable between species without losing function. Because plants have common ancestors, many genes in any plant species will have a corresponding orthologous gene in another plant species. Once a phylogenic tree for a gene family of one species has been constructed using a program such as CLUSTAL (Thompson et al. (1994) Nucleic Acids Res. 22: 4673-4680; Higgins et al. (1996) supra) potential orthologous sequences can be placed into the phylogenetic tree and their relationship to genes from the species of interest can be determined. Orthologous sequences can also be identified by a reciprocal BLAST strategy. Once an orthologous sequence has been identified, the function of the ortholog can be deduced from the identified function of the reference sequence.

Orthologous genes from different organisms have highly conserved functions, and very often essentially identical functions (Lee et al. (2002) Genome Res. 12: 493-502; Remm et al. (2001) J. Mol. Biol. 314: 1041-1052). Paralogous genes, which have diverged through gene duplication, may retain similar functions of the encoded proteins. In such cases, paralogs can be used interchangeably with respect to certain embodiments of the disclosure.

Those skilled in the art may also find further candidate CYP710A genes based on genome synteny and sequence similarity. In one embodiment, additional gene candidates can be obtained by hybridization or PCR using sequences based on the CYP710A nucleotide or amino acid sequences disclosed herein.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art. See, for example, Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York).

In hybridization techniques, all or part of a known polynucleotide is used as a probe that selectively hybridizes to other corresponding polynucleotides present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, N.Y.).

By “hybridizing to” or “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes part I chapter 2 “Overview of principles of hybridization and the strategy of nucleic acid probe assays” Elsevier, New York. Generally, highly stringent hybridization and wash conditions are selected to be about 5° C. lower than the thermal melting point (T m) for the specific sequence at a defined ionic strength and pH. Typically, under “stringent conditions” a probe will hybridize to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

The following are examples of sets of hybridization/wash conditions that may be used to clone nucleotide sequences that are homologues of reference nucleotide sequences: a reference nucleotide sequence preferably hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C.

### Reducing Expression or Activity of a CYP710A Gene

Several embodiments of the disclosure relate to reducing expression of an endogenous CYP710A gene in a plant. As used herein “reduced,” “reduction,” or the like refers to any detectable decrease in an experimental group (e.g., maize plant with a targeted DNA modification or a silencing element described herein) as compared to a control group (e.g., wild-type maize plant that does not comprise the targeted DNA modification or the silencing element). Methods for reducing expression of genes or gene products are well documented in the art.

In certain embodiments of the present disclosure, the expression or activity of the target gene is decreased or eliminated by disrupting the gene encoding the polypeptide. The gene encoding the polypeptide may be disrupted by any method known in the art, for example, by genome editing, transposon tagging, or mutagenizing plants using random or targeted mutagenesis and selecting for plants that have decreased expression activity.

### Genome Editing

In certain embodiments, the CYP710A target gene is modified using genome editing technology. Targeted modification of plant genomes through the use of genome editing methods can be used to reduce expression of a CYP710A gene through modification of plant genomic DNA. Genome editing methods can enable targeted insertion of one or more nucleic acids of interest into a plant genome. Genome editing uses engineered nucleases such as RNA guided DNA endonucleases or nucleases composed of sequence specific DNA binding domains fused to a non-specific DNA cleavage module. These engineered nucleases enable efficient and precise genetic modifications by inducing targeted DNA double stranded breaks that stimulate the cell's endogenous cellular DNA repair mechanisms to repair the induced break. Such mechanisms include, for example, error prone non-homologous end joining (NHEJ) and homology directed repair (HDR).

“Targeted DNA modification” can be used synonymously with targeted DNA mutation and refers to the introduction of a site specification modification that alters or changes the nucleotide sequence at a specific genomic locus of the plant (e.g., maize).

The targeted DNA modification described herein may be any modification known in the art such as, for example, insertion, deletion, single nucleotide polymorphism (SNP), and or a polynucleotide modification. Additionally, the targeted DNA modification in the genomic locus may be located anywhere in the genomic locus, such as, for example, a coding region of the encoded polypeptide (e.g., exon), a non-coding region (e.g., intron), a regulatory element, or untranslated region.

The type and location of the targeted DNA modification of the polynucleotide is not particularly limited so long as the targeted DNA modification results in reduced expression or activity of the protein encoded by the polynucleotide. In certain embodiments the targeted DNA modification is a deletion of one or more nucleotides, preferably contiguous, of the genomic locus.

In certain embodiments, a reduction in the expression or activity of the protein encoded by the polynucleotide is due to a targeted DNA modification at a genomic locus of a plant that results in one or more of the following: (a) reduced expression of the polynucleotide; (b) reduced transcriptional activity of the protein encoded by the polynucleotide; (c) generation of one or more alternatively spliced transcripts of the polynucleotide; (d) frameshift mutation in one or more exons of the polynucleotide; (e) deletion of a substantial portion of the polynucleotide or deletion of the full open reading frame of the polynucleotide; (f) repression of an enhancer motif present within a regulatory region encoding the polynucleotide; or (g) modification of one or more nucleotides or deletion of a regulatory element operably linked to the expression of the polynucleotide wherein the regulatory element is present within a promoter, intron, 3′UTR, terminator or a combination thereof.

In certain embodiments, the genomic locus has more than one (e.g., 2, 3, 4, 5) targeted DNA modification. For example, the translated region and a regulatory element of a genomic locus may each comprise a targeted DNA modification. In certain embodiments, the plant may have targeted DNA modifications at more than one (e.g., 2, 3, 4, 5, 6, 7, 8, 9, or 10) genomic loci comprising a CYP710A gene.

The targeted DNA modification of the genomic locus may be done using any genome modification technique known in the art. In certain embodiments, the targeted DNA modification is through a genome modification technique selected from the group consisting of a polynucleotide-guided endonuclease, CRISPR-Cas endonucleases, base editing deaminases, zinc finger nuclease, a transcription activator-like effector nuclease (TALEN), engineered site-specific meganuclease, or Argonaute.

In certain embodiments, the genome modification may be facilitated through the induction of a double-stranded break (DSB) or single-strand break, in a defined position in the genome near the desired alteration. DSB s can be induced using any DSB-inducing agent available, including, but not limited to, TALENs, meganucleases, zinc finger nucleases, Cas9-gRNA systems (based on bacterial CRISPR-Cas systems), guided cpf1 endonuclease systems, and the like. In certain embodiments, the introduction of a DSB can be combined with the introduction of a polynucleotide modification template.

A polynucleotide modification template can be introduced into a cell by any method known in the art, such as, but not limited to, transient introduction methods, transfection, electroporation, microinjection, particle mediated delivery, topical application, whiskers mediated delivery, delivery via cell-penetrating peptides, or mesoporous silica nanoparticle (MSN)-mediated direct delivery.

The polynucleotide modification template can be introduced into a cell as a single stranded polynucleotide molecule, a double stranded polynucleotide molecule, or as part of a circular DNA (vector DNA). The polynucleotide modification template can also be tethered to the guide RNA and/or the Cas endonuclease. Tethered DNAs can allow for co-localizing target and template DNA, useful in genome editing and targeted genome regulation, and can also be useful in targeting post-mitotic cells where function of endogenous HR machinery is expected to be highly diminished (Mali et al. 2013 Nature Methods Vol. 10: 957-963.) The polynucleotide modification template may be present transiently in the cell or it can be introduced via a viral replicon.

A “modified nucleotide” or “edited nucleotide” refers to a nucleotide sequence of interest that comprises at least one alteration when compared to its non-modified nucleotide sequence. Such “alterations” include, for example: (i) replacement of at least one nucleotide, (ii) a deletion of at least one nucleotide, (iii) an insertion of at least one nucleotide, or (iv) any combination of (i)-(iii).

The term “polynucleotide modification template” includes a polynucleotide that comprises at least one nucleotide modification when compared to the nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

The process for editing a genomic sequence combining DSB and modification templates generally comprises: providing to a host cell, a DSB-inducing agent, or a nucleic acid encoding a DSB-inducing agent, that recognizes a target sequence in the chromosomal sequence and is able to induce a DSB in the genomic sequence, and at least one polynucleotide modification template comprising at least one nucleotide alteration when compared to the nucleotide sequence to be edited. The polynucleotide modification template can further comprise nucleotide sequences flanking the at least one nucleotide alteration, in which the flanking sequences are substantially homologous to the chromosomal region flanking the DSB.

The endonuclease can be provided to a cell by any method known in the art, for example, but not limited to, transient introduction methods, transfection, microinjection, and/or topical application or indirectly via recombination constructs. The endonuclease can be provided as a protein or as a guided polynucleotide complex directly to a cell or indirectly via recombination constructs. The endonuclease can be introduced into a cell transiently or can be incorporated into the genome of the host cell using any method known in the art. In the case of a CRISPR-Cas system, uptake of the endonuclease and/or the guided polynucleotide into the cell can be facilitated with a Cell Penetrating Peptide (CPP) as described in WO2016073433 published May 12, 2016.

As used herein, a “genomic region” is a segment of a chromosome in the genome of a cell that is present on either side of the target site or, alternatively, also comprises a portion of the target site. The genomic region can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800. 5-2900, 5-3000, 5-3100 or more bases such that the genomic region has sufficient homology to undergo homologous recombination with the corresponding region of homology.

TAL effector nucleases (TALEN) are a class of sequence-specific nucleases that can be used to make double-strand breaks at specific target sequences in the genome of a plant or other organism. (Miller et al. (2011) Nature Biotechnology 29:143-148).

A TALEN comprises a TAL effector DNA binding domain and an endonuclease domain. TAL effectors are proteins of plant pathogenic bacteria that are injected by the pathogen into the plant cell, where they travel to the nucleus and function as transcription factors to turn on specific plant genes. The primary amino acid sequence of a TAL effector dictates the nucleotide sequence to which it binds. Thus, target sites can be predicted for TAL effectors, and TAL effectors can be engineered and generated for the purpose of binding to particular nucleotide sequences.

Fused to the TAL effector-encoding nucleic acid sequences are sequences encoding a nuclease or a portion of a nuclease, typically a nonspecific cleavage domain from a type II restriction endonuclease such as FokI (Kim et al., 1996). Other useful endonucleases may include, for example, HhaI, HindIII, Nod, BbvCI, EcoRI, BgII, and AhvI. The fact that some endonucleases (e.g., FokI) only function as dimers can be capitalized upon to enhance the target specificity of the TAL effector. For example, in some cases each FokI monomer can be fused to a TAL effector sequence that recognizes a different DNA target sequence, and only when the two recognition sites are in close proximity do the inactive monomers come together to create a functional enzyme. By requiring DNA binding to activate the nuclease, a highly site-specific restriction enzyme can be created.

Endonucleases are enzymes that cleave the phosphodiester bond within a polynucleotide chain. Endonucleases include restriction endonucleases, which cleave DNA at specific sites without damaging the bases, and meganucleases, also known as homing endonucleases (HEases), which like restriction endonucleases, bind and cut at a specific recognition site, however the recognition sites for meganucleases are typically longer, about 18 bp or more (patent application PCT/US12/30061, filed on Mar. 22, 2012). Meganucleases have been classified into four families based on conserved sequence motifs, the families are the LAGLIDADG, GIY-YIG, H-N-H, and His-Cys box families. These motifs participate in the coordination of metal ions and hydrolysis of phosphodiester bonds. HEases are notable for their long recognition sites, and for tolerating some sequence polymorphisms in their DNA substrates. The naming convention for meganuclease is similar to the convention for other restriction endonuclease. Meganucleases are also characterized by prefix F-, I-, or Pl- for enzymes encoded by free-standing ORFs, introns, and inteins, respectively. One step in the recombination process involves polynucleotide cleavage at or near the recognition site. The cleaving activity can be used to produce a double-strand break. For reviews of site-specific recombinases and their recognition sites, see, Sauer (1994) Curr Op Biotechnol 5:521-7; and Sadowski (1993) FASEB 7:760-7. In some examples the recombinase is from the Integrase or Resolvase families.

Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered. Zinc finger domains are amenable for designing polypeptides which specifically bind a selected polynucleotide recognition sequence. ZFNs include an engineered DNA-binding zinc finger domain linked to a non-specific endonuclease domain, for example nuclease domain from a Type IIs endonuclease such as FokI. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3 finger domain recognized a sequence of 9 contiguous nucleotides, with a dimerization requirement of the nuclease, two sets of zinc finger triplets are used to bind an 18 nucleotide recognition sequence.

Genome editing using DSB-inducing agents, such as Cas9-gRNA complexes, has been described, for example in U.S. Patent Application US 2015-0082478 A1, published on Mar. 19, 2015, WO2015/026886 A1, published on Feb. 26, 2015, WO2016007347, published on Jan. 14, 2016, and WO201625131, published on Feb. 18, 2016, all of which are incorporated by reference herein.

The term “Cas gene” herein refers to a gene that is generally coupled, associated or close to, or in the vicinity of flanking CRISPR loci in bacterial systems. The terms “Cas gene”, “CRISPR-associated (Cas) gene” are used interchangeably herein. The term “Cas endonuclease” herein refers to a protein encoded by a Cas gene. A Cas endonuclease herein, when in complex with a suitable polynucleotide component, is capable of recognizing, binding to, and optionally nicking or cleaving all or part of a specific DNA target sequence. A Cas endonuclease described herein comprises one or more nuclease domains. Cas endonucleases of the disclosure includes those having a HNH or HNH-like nuclease domain and/or a RuvC or RuvC-like nuclease domain. A Cas endonuclease of the disclosure include, for example a Cas9 protein, a Cas12a protein, a Cas12b protein, or complexes of these.

As used herein, the terms “guide polynucleotide/Cas endonuclease complex”, “guide polynucleotide/Cas endonuclease system”, “guide polynucleotide/Cas complex”, “guide polynucleotide/Cas system”, “guided Cas system” are used interchangeably herein and refer to at least one guide polynucleotide and at least one Cas endonuclease that are capable of forming a complex, wherein said guide polynucleotide/Cas endonuclease complex can direct the Cas endonuclease to a DNA target site, enabling the Cas endonuclease to recognize, bind to, and optionally nick or cleave (introduce a single or double strand break) the DNA target site. A guide polynucleotide/Cas endonuclease complex herein can comprise Cas protein(s) and suitable polynucleotide component(s) of any of the four known CRISPR systems (Horvath and Barrangou, 2010, Science 327:167-170) such as a type I, II, or III CRISPR system. A Cas endonuclease unwinds the DNA duplex at the target sequence and optionally cleaves at least one DNA strand, as mediated by recognition of the target sequence by a polynucleotide (such as, but not limited to, a crRNA or guide RNA) that is in complex with the Cas protein. Such recognition and cutting of a target sequence by a Cas endonuclease typically occurs if the correct protospacer-adjacent motif (PAM) is located at or adjacent to the 3′ end of the DNA target sequence. Alternatively, a Cas protein herein may lack DNA cleavage or nicking activity, but can still specifically bind to a DNA target sequence when complexed with a suitable RNA component. (See also U.S. Patent Application US 2015-0082478 A1, published on Mar. 19, 2015 and US 2015-0059010 A1, published on Feb. 26, 2015, both are hereby incorporated in its entirety by reference).

A guide polynucleotide/Cas endonuclease complex can cleave one or both strands of a DNA target sequence. A guide polynucleotide/Cas endonuclease complex that can cleave both strands of a DNA target sequence typically comprise a Cas protein that has all of its endonuclease domains in a functional state (e.g., wild type endonuclease domains or variants thereof retaining some or all activity in each endonuclease domain). Non-limiting examples of Cas9 nickases suitable for use herein are disclosed in U.S. Patent Appl. Publ. No. 2014/0189896, which is incorporated herein by reference.

Other Cas endonuclease systems have been described in PCT patent applications PCT/US16/32073, filed May 12, 2016 and PCT/US16/32028 filed May 12, 2016, both applications incorporated herein by reference.

“Cas9” (formerly referred to as Cas5, Csn1, or Csx12) herein refers to a Cas endonuclease of a type II CRISPR system that forms a complex with a crNucleotide and a tracrNucleotide, or with a single guide polynucleotide, for specifically recognizing and cleaving all or part of a DNA target sequence. Cas9 protein comprises a RuvC nuclease domain and an HNH (H-N-H) nuclease domain, each of which can cleave a single DNA strand at a target sequence (the concerted action of both domains leads to DNA double-strand cleavage, whereas activity of one domain leads to a nick). In general, the RuvC domain comprises subdomains I, II and III, where domain I is located near the N-terminus of Cas9 and subdomains II and III are located in the middle of the protein, flanking the HNH domain (Hsu et al, Cell 157:1262-1278). A type II CRISPR system includes a DNA cleavage system utilizing a Cas9 endonuclease in complex with at least one polynucleotide component. For example, a Cas9 can be in complex with a CRISPR RNA (crRNA) and a trans-activating CRISPR RNA (tracrRNA). In another example, a Cas9 can be in complex with a single guide RNA.

Any guided endonuclease can be used in the methods disclosed herein. Such endonucleases include, but are not limited to Cas9, Cas12a, and Cas12b endonucleases. Many endonucleases have been described to date that can recognize specific PAM sequences (see for example—Jinek et al. (2012) Science 337 p 816-821, PCT patent applications PCT/US16/32073, filed May 12, 2016 and PCT/US16/32028 filed May 12, 2016 and Zetsche B et al. 2015. Cell 163, 1013) and cleave the target DNA at a specific position. It is understood that based on the methods and embodiments described herein utilizing a guided Cas system one can now tailor these methods such that they can utilize any guided endonuclease system.

The guide polynucleotide can also be a single molecule (also referred to as single guide polynucleotide) comprising a crRNA sequence linked to a tracrRNA sequence. The single guide polynucleotide comprises a first nucleotide sequence domain (referred to as Variable Targeting domain or VT domain) that can hybridize to a nucleotide sequence in a target DNA and a Cas endonuclease recognition domain (CER domain), that interacts with a Cas endonuclease polypeptide. By “domain” it is meant a contiguous stretch of nucleotides that can be RNA, DNA, and/or RNA-DNA-combination sequence. The VT domain and/or the CER domain of a single guide polynucleotide can comprise a RNA sequence, a DNA sequence, or an RNA-DNA-combination sequence. The single guide polynucleotide being comprised of sequences from the crRNA and the tracrRNA may be referred to as “single guide RNA” (when composed of a contiguous stretch of RNA nucleotides) or “single guide DNA” (when composed of a contiguous stretch of DNA nucleotides) or “single guide RNA-DNA” (when composed of a combination of RNA and DNA nucleotides). The single guide polynucleotide can form a complex with a Cas endonuclease, wherein said guide polynucleotide/Cas endonuclease complex (also referred to as a guide polynucleotide/Cas endonuclease system) can direct the Cas endonuclease to a genomic target site, enabling the Cas endonuclease to recognize, bind to, and optionally nick or cleave (introduce a single or double strand break) the target site. (See also U.S. Patent Application US 2015-0082478 A1, published on Mar. 19, 2015 and US 2015-0059010 A1, published on Feb. 26, 2015, both are hereby incorporated in its entirety by reference.)

The term “variable targeting domain” or “VT domain” is used interchangeably herein and includes a nucleotide sequence that can hybridize (is complementary) to one strand (nucleotide sequence) of a double strand DNA target site. In certain embodiments, the variable targeting domain comprises a contiguous stretch of 12 to 30 nucleotides. The variable targeting domain can be composed of a DNA sequence, a RNA sequence, a modified DNA sequence, a modified RNA sequence, or any combination thereof.

The terms “single guide RNA” and “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA). The single guide RNA can comprise a crRNA or crRNA fragment and a tracrRNA or tracrRNA fragment of the type II CRISPR/Cas system that can form a complex with a type II Cas endonuclease, wherein said guide RNA/Cas endonuclease complex can direct the Cas endonuclease to a DNA target site, enabling the Cas endonuclease to recognize, bind to, and optionally nick or cleave (introduce a single or double strand break) the DNA target site.

The terms “guide RNA/Cas endonuclease complex”, “guide RNA/Cas endonuclease system”, “guide RNA/Cas complex”, “guide RNA/Cas system”, “gRNA/Cas complex”, “gRNA/Cas system”, “RNA-guided endonuclease”, “RGEN” are used interchangeably herein and refer to at least one RNA component and at least one Cas endonuclease that are capable of forming a complex, wherein said guide RNA/Cas endonuclease complex can direct the Cas endonuclease to a DNA target site, enabling the Cas endonuclease to recognize, bind to, and optionally nick or cleave (introduce a single or double strand break) the DNA target site. A guide RNA/Cas endonuclease complex herein can comprise Cas protein(s) and suitable RNA component(s) of any of the four known CRISPR systems (Horvath and Barrangou, 2010, Science 327:167-170) such as a type I, II, or III CRISPR system. A guide RNA/Cas endonuclease complex can comprise a Type II Cas9 endonuclease and at least one RNA component (e.g., a crRNA and tracrRNA, or a gRNA). (See also U.S. Patent Application US 2015-0082478 A1, published on Mar. 19, 2015 and US 2015-0059010 A1, published on Feb. 26, 2015, both are hereby incorporated in its entirety by reference).

The guide polynucleotide of the methods and compositions described herein may be any polynucleotide sequence that targets the genomic loci of a plant cell comprising a polynucleotide that encodes an amino acid sequence that has at least 80%, at least 90%, at least 95%, at least 96%, at least 97%, at least 98%, or at least 99% identical to SEQ ID NO: 1. In certain embodiments, the guide polynucleotide is a guide RNA. The guide polynucleotide may also be present in a recombinant DNA construct.

The guide polynucleotide can be introduced into a cell transiently, as single stranded polynucleotide or a double stranded polynucleotide, using any method known in the art such as, but not limited to, particle bombardment, Agrobacterium transformation or topical applications. The guide polynucleotide can also be introduced indirectly into a cell by introducing a recombinant DNA molecule (via methods such as, but not limited to, particle bombardment or Agrobacterium transformation) comprising a heterologous nucleic acid fragment encoding a guide polynucleotide, operably linked to a specific promoter that is capable of transcribing the guide RNA in said cell. The specific promoter can be, but is not limited to, a RNA polymerase III promoter, which allow for transcription of RNA with precisely defined, unmodified, 5′- and 3′-ends (DiCarlo et al., Nucleic Acids Res. 41: 4336-4343; Ma et al., Mol. Ther. Nucleic Acids 3:e161) as described in WO2016025131, published on Feb. 18, 2016, incorporated herein in its entirety by reference.

The terms “target site”, “target sequence”, “target site sequence, “target DNA”, “target locus”, “genomic target site”, “genomic target sequence”, “genomic target locus” and “protospacer”, are used interchangeably herein and refer to a polynucleotide sequence such as, but not limited to, a nucleotide sequence on a chromosome, episome, or any other DNA molecule in the genome (including chromosomal, chloroplastic, mitochondrial DNA, plasmid DNA) of a cell, at which a guide polynucleotide/Cas endonuclease complex can recognize, bind to, and optionally nick or cleave. The target site can be an endogenous site in the genome of a cell, or alternatively, the target site can be heterologous to the cell and thereby not be naturally occurring in the genome of the cell, or the target site can be found in a heterologous genomic location compared to where it occurs in nature. As used herein, terms “endogenous target sequence” and “native target sequence” are used interchangeable herein to refer to a target sequence that is endogenous or native to the genome of a cell and is at the endogenous or native position of that target sequence in the genome of the cell. Cells include, but are not limited to, human, non-human, animal, bacterial, fungal, insect, yeast, non-conventional yeast, and plant cells as well as plants and seeds produced by the methods described herein. An “artificial target site” or “artificial target sequence” are used interchangeably herein and refer to a target sequence that has been introduced into the genome of a cell. Such an artificial target sequence can be identical in sequence to an endogenous or native target sequence in the genome of a cell but be located in a different position (i.e., a non-endogenous or non-native position) in the genome of a cell.

An “altered target site”, “altered target sequence”, “modified target site”, “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

Methods for “modifying a target site” and “altering a target site” are used interchangeably herein and refer to methods for producing an altered target site.

The length of the target DNA sequence (target site) can vary, and includes, for example, target sites that are at least 12, 13, 14, 15, 16, 17, 18, 19, 20, 21, 22, 23, 24, 25, 26, 27, 28, 29, 30 or more nucleotides in length. It is further possible that the target site can be palindromic, that is, the sequence on one strand reads the same in the opposite direction on the complementary strand. The nick/cleavage site can be within the target sequence or the nick/cleavage site could be outside of the target sequence. In another variation, the cleavage could occur at nucleotide positions immediately opposite each other to produce a blunt end cut or, in other cases, the incisions could be staggered to produce single-stranded overhangs, also called “sticky ends”, which can be either 5′ overhangs, or 3′ overhangs. Active variants of genomic target sites can also be used. Such active variants can comprise at least 65%, 70%, 75%, 80%, 85%, 90%, 91%, 92%, 93%, 94%, 95%, 96%, 97%, 98%, 99% or more sequence identity to the given target site, wherein the active variants retain biological activity and hence are capable of being recognized and cleaved by a Cas endonuclease. Assays to measure the single or double-strand break of a target site by an endonuclease are known in the art and generally measure the overall activity and specificity of the agent on DNA substrates containing recognition sites.

A “protospacer adjacent motif” (PAM) herein refers to a short nucleotide sequence adjacent to a target sequence (protospacer) that is recognized (targeted) by a guide polynucleotide/Cas endonuclease system described herein. The Cas endonuclease may not successfully recognize a target DNA sequence if the target DNA sequence is not followed by a PAM sequence. The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long.

The terms “targeting”, “gene targeting” and “DNA targeting” are used interchangeably herein. DNA targeting herein may be the specific introduction of a knock-out, edit, or knock-in at a particular DNA sequence, such as in a chromosome or plasmid of a cell. In general, DNA targeting can be performed herein by cleaving one or both strands at a specific DNA sequence in a cell with an endonuclease associated with a suitable polynucleotide component. Such DNA cleavage, if a double-strand break (DSB), can prompt NHEJ or HDR processes which can lead to modifications at the target site.

A targeting method herein can be performed in such a way that two or more DNA target sites are targeted in the method, for example. Such a method can optionally be characterized as a multiplex method. Two, three, four, five, six, seven, eight, nine, ten, or more target sites can be targeted at the same time in certain embodiments. A multiplex method is typically performed by a targeting method herein in which multiple different RNA components are provided, each designed to guide an guide polynucleotide/Cas endonuclease complex to a unique DNA target site.

The guide polynucleotide/Cas endonuclease system can be used in combination with a co-delivered polynucleotide modification template to allow for editing (modification) of a genomic nucleotide sequence of interest. (See also U.S. Patent Application US 2015-0082478 A1, published on Mar. 19, 2015 and WO2015/026886 A1, published on Feb. 26, 2015, both are hereby incorporated in its entirety by reference.)

Various methods and compositions can be employed to obtain a cell or organism having a polynucleotide of interest inserted in a target site. Such methods can employ homologous recombination to provide integration of the polynucleotide of Interest at the target site. In one method provided, a polynucleotide of interest is provided to the organism cell in a donor DNA construct. As used herein, “donor DNA” is a DNA construct that comprises a polynucleotide of Interest to be inserted into the target site. The donor DNA construct further comprises a first and a second region of homology that flank the polynucleotide of Interest. The first and second regions of homology of the donor DNA share homology to a first and a second genomic region, respectively, present in or flanking the target site of the cell or organism genome. By “homology” is meant DNA sequences that are similar. For example, a “region of homology to a genomic region” that is found on the donor DNA is a region of DNA that has a similar sequence to a given “genomic region” in the cell or organism genome. A region of homology can be of any length that is sufficient to promote homologous recombination at the cleaved target site. For example, the region of homology can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800, 5-2900, 5-3000, 5-3100 or more bases in length such that the region of homology has sufficient homology to undergo homologous recombination with the corresponding genomic region. “Sufficient homology” indicates that two polynucleotide sequences have sufficient structural similarity to act as substrates for a homologous recombination reaction. The structural similarity includes overall length of each polynucleotide fragment, as well as the sequence similarity of the polynucleotides. Sequence similarity can be described by the percent sequence identity over the whole length of the sequences, and/or by conserved regions comprising localized similarities such as contiguous nucleotides having 100% sequence identity, and percent sequence identity over a portion of the length of the sequences.

The amount of sequence identity shared by a target and a donor polynucleotide can vary and includes total lengths and/or regions having unit integral values in the ranges of about 1-20 bp, 20-50 bp, 50-100 bp, 75-150 bp, 100-250 bp, 150-300 bp, 200-400 bp, 250-500 bp, 300-600 bp, 350-750 bp, 400-800 bp, 450-900 bp, 500-1000 bp, 600-1250 bp, 700-1500 bp, 800-1750 bp, 900-2000 bp, 1-2.5 kb, 1.5-3 kb, 2-4 kb, 2.5-5 kb, 3-6 kb, 3.5-7 kb, 4-8 kb, 5-10 kb, or up to and including the total length of the target site. These ranges include every integer within the range, for example, the range of 1-20 bp includes 1, 2, 3, 4, 5, 6, 7, 8, 9, 10, 11, 12, 13, 14, 15, 16, 17, 18, 19 and 20 bps. The amount of homology can also be described by percent sequence identity over the full aligned length of the two polynucleotides which includes percent sequence identity of about at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Sufficient homology includes any combination of polynucleotide length, global percent sequence identity, and optionally conserved regions of contiguous nucleotides or local percent sequence identity, for example sufficient homology can be described as a region of 75-150 bp having at least 80% sequence identity to a region of the target locus. Sufficient homology can also be described by the predicted ability of two polynucleotides to specifically hybridize under high stringency conditions, see, for example, Sambrook et al., (1989) Molecular Cloning: A Laboratory Manual, (Cold Spring Harbor Laboratory Press, NY); Current Protocols in Molecular Biology, Ausubel et al., Eds (1994) Current Protocols, (Greene Publishing Associates, Inc. and John Wiley & Sons, Inc.); and, Tijssen (1993) Laboratory Techniques in Biochemistry and Molecular Biology-Hybridization with Nucleic Acid Probes, (Elsevier, New York).

The structural similarity between a given genomic region and the corresponding region of homology found on the donor DNA can be any degree of sequence identity that allows for homologous recombination to occur. For example, the amount of homology or sequence identity shared by the “region of homology” of the donor DNA and the “genomic region” of the organism genome can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination

The region of homology on the donor DNA can have homology to any sequence flanking the target site. While in certain embodiments the regions of homology share significant sequence homology to the genomic sequence immediately flanking the target site, it is recognized that the regions of homology can be designed to have sufficient homology to regions that may be further 5′ or 3′ to the target site. In still other embodiments, the regions of homology can also have homology with a fragment of the target site along with downstream genomic regions. In one embodiment, the first region of homology further comprises a first fragment of the target site and the second region of homology comprises a second fragment of the target site, wherein the first and second fragments are dissimilar.

As used herein, “homologous recombination” includes the exchange of DNA fragments between two DNA molecules at the sites of homology.

Further uses for guide RNA/Cas endonuclease systems have been described (See U.S. Patent Application US 2015-0082478 A1, published on Mar. 19, 2015, WO2015/026886 A1, published on Feb. 26, 2015, US 2015-0059010 A1, published on Feb. 26, 2015, U.S. application 62/023,246, filed on Jul. 7, 2014, and U.S. application 62/036,652, filed on Aug. 13, 2014, all of which are incorporated by reference herein) and include but are not limited to modifying or replacing nucleotide sequences of interest (such as a regulatory elements), insertion of polynucleotides of interest, gene knock-out, gene-knock in, modification of splicing sites and/or introducing alternate splicing sites, modifications of nucleotide sequences encoding a protein of interest, amino acid and/or protein fusions, and gene silencing by expressing an inverted repeat into a gene of interest.

### Transposon Tagging

In certain embodiments, transposon tagging is used to decrease or eliminate the activity of one or more polypeptides. Transposon tagging comprises inserting a transposon within an endogenous gene in the pathway to decrease or eliminate expression of the polypeptide.

In this embodiment, the expression of one or more polypeptides is decreased or eliminated by inserting a transposon within a regulatory region or coding region of the gene encoding the polypeptide. A transposon that is within an exon, intron, 5′ or 3′ untranslated sequence, a promoter or any other regulatory sequence of a gene may be used to decrease or eliminate the expression and/or activity of the encoded polypeptide.

Methods for the transposon tagging of specific genes in plants are well known in the art. See, e.g., Maes et al., (1999) Trends Plant Sci. 4:90-96; Dharmapuri & Sonti, (1999) FEMS Microbiol. Lett. 179:53-59; Meissner et al., (2000) Plant J. 22:265-74; Phogat et al., (2000) J. Biosci. 25:57-63; Walbot, (2000) Curr. Opin. Plant Biol. 2:103-07; Gai et al., (2000) Nucleic Acids Res. 28:94-96; Fitzmaurice et al., (1999) Genetics 153:1919-28. In addition, the TUSC process for selecting Mu insertions in selected genes has been described in Bensen et al., (1995) Plant Cell 7:75-84; Mena et al., (1996) Science 274:1537-40; and U.S. Pat. No. 5,962,764, each of which is herein incorporated by reference.

### Induced Mutagenesis

Additional methods for reducing or eliminating the expression of endogenous CYP710A genes in plants are also known in the art and may be similarly applied to the instant disclosure. These methods include other forms of mutagenesis, such as ethyl methanesulfonate-induced mutagenesis, deletion mutagenesis, and fast neutron deletion mutagenesis used in a reverse genetics sense (with PCR) to identify plant lines, in which the endogenous gene has been mutated or deleted. For examples of these methods see Ohshima et al., (1998) Virology 243:472-81; Okubara et al., (1994) Genetics 137:867-74; and Quesada et al., (2000) Genetics 154:421-36, each of which is herein incorporated by reference. In addition, a fast and automatable method for screening for chemically induced mutations, TILLING (Targeting Induced Local Lesions In Genomes), using denaturing HPLC or selective endonuclease digestion of selected PCR products is also applicable to the instant disclosure. See McCallum et al., (2000) Nat. Biotechnol. 18:455-57, herein incorporated by reference.

Mutations may impact gene expression or interfere with the activity of an encoded CYP710A protein. Insertional mutations in gene exons usually result in null-mutants. Mutations in conserved residues are particularly effective in inhibiting the activity of the encoded protein. Conserved residues of plant polypeptides suitable for mutagenesis with the goal to eliminate activity have been described. Such mutants may be isolated according to well-known procedures and mutations in different CYP710A target gene loci may be stacked by genetic crossing. See, e.g., Gruis et al., (2002) Plant Cell 14:2863-82.

In certain embodiments, dominant mutants are used to trigger RNA silencing due to gene inversion and recombination of a duplicated gene locus. See, e.g., Kusaba et al., (2003) Plant Cell 15:1455-67.

The disclosure encompasses additional methods for decreasing or eliminating the activity of one or more CYP710A target polypeptides. Examples of other methods for altering or mutating a genomic nucleotide sequence in a plant are known in the art and include, but are not limited to, the use of RNA:DNA vectors, RNA:DNA mutational vectors, RNA:DNA repair vectors, mixed-duplex oligonucleotides, self-complementary RNA:DNA oligonucleotides, and recombinogenic oligonucleobases. Such vectors and methods of use are known in the art. See, for example, U.S. Pat. Nos. 5,565,350; 5,731,181; 5,756,325; 5,760,012; 5,795,972; and 5,871,984, each of which are herein incorporated by reference. See also WO 1998/49350, WO 1999/07865, WO 1999/25821, and Beetham et al., (1999) Proc. Natl. Acad. Sci. USA 96:8774-78, each of which is herein incorporated by reference.

### Silencing Elements

As used herein, “silencing element” refers to a polynucleotide that is capable of reducing or eliminating the level or expression of a target polynucleotide or the polypeptide encoded thereby. The silencing element employed can reduce or eliminate the expression level of the target sequence by influencing the level of the target RNA transcript or, alternatively, by influencing translation and thereby affecting the level of the encoded polypeptide. A single polynucleotide employed in the methods can comprise one or more silencing elements to the same or different target polynucleotides. The silencing element can be produced in vivo (i.e., in a host cell such as a plant) or in vitro.

In certain embodiments, reducing the CYP710A target gene expression or activity comprises introducing into a plant or plant cell a silencing element, thereby reducing or eliminating the level or expression of a polynucleotide or a polypeptide encoded by the target gene.

Non-limiting examples of silencing elements include, a sense suppression element, an antisense suppression element, a double stranded RNA, a siRNA, an amiRNA, a miRNA, or a hairpin suppression element. Non-limiting examples of silencing elements that can be employed to decrease expression of these target sequences or additionally sequences targeting genes involved in recombination comprise fragments and variants of the sense or antisense sequence or consists of the sense or antisense sequence of wild type polynucleotide or polypeptide sequences, variant polynucleotides, variant polypeptides, cognate promoter sequences, ortholog sequences, variants or fragments thereof. The silencing element can further comprise additional sequences that advantageously effect transcription and/or the stability of a resulting transcript. For example, the silencing elements can comprise at least one thymine residue at the 3′ end. This can aid in stabilization. Thus, the silencing elements can have at least 1, 2, 3, 4, 5, 6, 7, 8, 9, 10 or more thymine residues at the 3′ end. Enhancer suppressor elements can also be employed in conjunction with the silencing elements.

In certain embodiments, introducing the silencing element reduces the polynucleotide level and/or the polypeptide level of the target sequence to less than 95%, less than 90%, less than 80%, less than 70%, less than 60%, less than 50%, less than 40%, less than 30%, less than 20%, less than 10%, or less than 5% of the polynucleotide level, or the level of the polypeptide encoded thereby, of the same target sequence in an appropriate control.

In certain embodiments, decreasing expression of the CYP710A target gene is obtained by sense suppression or cosuppression. For cosuppression, an expression cassette is designed to express an RNA molecule corresponding to all or part of a messenger RNA encoding a polypeptide in the “sense” orientation. Over expression of the RNA molecule may result in decreased expression of the native gene. Accordingly, multiple plant lines transformed with the cosuppression expression cassette are screened to identify those that show the desired degree of inhibition of polypeptide expression.

Typically, a sense suppression element has substantial sequence identity to the target polynucleotide, typically greater than about 65% sequence identity, greater than about 85% sequence identity, about 90%, 91%, 92%, 93%, 94%, 95%, 96%, 97%, 98% or 99% sequence identity. See, U.S. Pat. Nos. 5,283,184 and 5,034,323; herein incorporated by reference. The sense suppression element can be any length so long as it allows for the suppression of the targeted sequence. In other implementations, the sense suppression element is, for example, 15, 16, 17, 18 19, 20, 22, 25, 30, 50, 100, 150, 200, 250, 300, 350, 400, 450, 500, 600, 700, 900, 1000, 1100, 1200, 1300 nucleotides or longer of the target polynucleotides. In other implementations, the sense suppression element is, for example, about 15-25, 25-100, 100-150, 150-200, 200-250, 250-300, 300-350, 350-400, 450-500, 500-550, 550-600, 600-650, 650-700, 700-750, 750-800, 800-850, 850-900, 900-950, 950-1000, 1000-1050, 1050-1100, 1100-1200, 1200-1300, 1300-1400, 1400-1500, 1500-1600, 1600-1700, 1700-1800 nucleotides or longer of the target polynucleotides.

The polynucleotide used for cosuppression may correspond to all or part of the sequence encoding the polypeptide, all or part of the 5′ and/or 3′ untranslated region of a polypeptide transcript or all or part of both the coding sequence and the untranslated regions of a transcript encoding a polypeptide. In certain embodiments where the polynucleotide comprises all or part of the coding region for the polypeptide, the expression cassette is designed to eliminate the start codon of the polynucleotide so that no protein product will be translated.

Cosuppression has been used to inhibit the expression of plant genes to produce plants having undetectable protein levels for the proteins encoded by these genes (see, e.g., Broin et al., (2002) Plant Cell 14:1417-32), and to inhibit the expression of multiple proteins in the same plant (see, e.g., U.S. Pat. No. 5,942,657). Methods for using cosuppression to inhibit the expression of endogenous genes in plants are described in Flavell et al., (1994) Proc. Natl. Acad. Sci. USA 91:3490-96; Jorgensen et al., (1996) Plant Mol. Biol. 31:957-73; Johansen & Carrington, (2001) Plant Physiol. 126:930-38; Broin et al., (2002) Plant Cell 14:1417-32; Stoutj esdij k et al., (2002) Plant Physiol. 129:1723-31; Yu et al., (2003) Phytochemistry 63: 753-63; and U.S. Pat. Nos. 5,034,323, 5,283,184, and 5,942,657, each of which is herein incorporated by reference. The efficiency of cosuppression may be increased by including a poly-dT region in the expression cassette at a position 3′ to the sense sequence and 5′ of the polyadenylation signal. See, US Patent Application Publication No. 2002/0048814, herein incorporated by reference. Typically, such a nucleotide sequence has substantial sequence identity to the sequence of the transcript of the endogenous gene, optimally greater than about 65% sequence identity, more optimally greater than about 85% sequence identity, most optimally greater than about 95% sequence identity. See U.S. Pat. Nos. 5,283,184 and 5,034,323, herein incorporated by reference.

In certain embodiments, decreasing expression of the CYP710A target gene is obtained by antisense suppression. For antisense suppression, the expression cassette is designed to express an RNA molecule complementary to all or part of a messenger RNA encoding the polypeptide. Over expression of the antisense RNA molecule may result in decreased expression of the target gene. Accordingly, multiple plant lines transformed with the antisense suppression expression cassette are screened to identify those that show the desired degree of inhibition of polypeptide expression.

The polynucleotide for use in antisense suppression may correspond to all or part of the complement of the sequence encoding the polypeptide, all or part of the complement of the 5′ and/or 3′ untranslated region of the target transcript or all or part of the complement of both the coding sequence and the untranslated regions of a transcript encoding the polypeptide. In addition, the antisense polynucleotide may be fully complementary (i.e., 100% identical to the complement of the target sequence) or partially complementary (i.e., less than 100% identical to the complement of the target sequence) to the target sequence. In addition, the antisense suppression element may be fully complementary (i.e., 100% identical to the complement of the target sequence) or partially complementary (i.e., less than 100% identical to the complement of the target sequence) to the target polynucleotide. In specific embodiments, the antisense suppression element comprises at least 85%, 90%, 91%, 92%, 93%, 94%, 95%, 96%, 97%, 98%, or 99% sequence complementarity to the target polynucleotide. Antisense suppression may be used to inhibit the expression of multiple proteins in the same plant. See, for example, U.S. Pat. No. 5,942,657. Furthermore, the antisense suppression element can be complementary to a portion of the target polynucleotide. Generally, sequences of at least 15, 20, 22, 25, 50, 100, 200, 300, 400, 450 nucleotides or greater of the target gene sequence may be used. Methods for using antisense suppression to inhibit the expression of endogenous genes in plants are described, e.g., in Liu et al. (2002) Plant Physiol. 129:1732-43 and U.S. Pat. Nos. 5,759,829 and 5,942,657, each of which is herein incorporated by reference. Efficiency of antisense suppression may be increased by including a poly-dT region in the expression cassette at a position 3′ to the antisense sequence and 5′ of the polyadenylation signal. See, US Patent Application Publication No. 2002/0048814, herein incorporated by reference.

In certain embodiments, decreasing the expression of the CYP710A target gene is obtained by double-stranded RNA (dsRNA) interference. A “double stranded RNA silencing element” or “dsRNA” comprises at least one transcript that is capable of forming a dsRNA. Thus, a “dsRNA silencing element” includes a dsRNA, a transcript or polyribonucleotide capable of forming a dsRNA or more than one transcript or polyribonucleotide capable of forming a dsRNA. “Double stranded RNA” or “dsRNA” refers to a polyribonucleotide structure formed either by a single self-complementary RNA molecule or a polyribonucleotide structure formed by the expression of least two distinct RNA strands. The dsRNA molecule(s) employed in the methods and compositions mediate the decrease of expression of a target sequence, for example, by mediating RNA interference “RNAi” or gene silencing in a sequence-specific manner. The dsRNA is capable of decreasing or eliminating the level or expression of a target polynucleotide or the polypeptide, for example, CYP710A.

The dsRNA can decrease or eliminate the expression level of the target sequence by influencing the level of the target RNA transcript, by influencing translation and thereby affecting the level of the encoded polypeptide, or by influencing expression at the pre-transcriptional level (i.e., via the modulation of chromatin structure, methylation pattern, etc., to alter gene expression). See, e.g., Verdel et al., (2004) Science 303:672-76; Pal-Bhadra et al., (2004) Science 303:669-72; All shire (2002) Science 297:1818-19; Volpe et al., (2002) Science 297:1833-37; Jenuwein (2002) Science 297:2215-18; and Hall et al., (2002) Science 297:2232-37. As used herein, the term “dsRNA” is meant to encompass other terms used to describe nucleic acid molecules that are capable of mediating RNA interference or gene silencing, including, e.g., short-interfering RNA (siRNA), double-stranded RNA (dsRNA), micro-RNA (miRNA), hairpin RNA, short hairpin RNA (shRNA), post-transcriptional gene silencing RNA (ptgsRNA), etc.

For dsRNA interference, a sense RNA molecule like that described above for cosuppression and an antisense RNA molecule that is fully or partially complementary to the sense RNA molecule are expressed in the same cell, resulting in inhibition of the expression of the corresponding endogenous messenger RNA.

Expression of the sense and antisense molecules may be accomplished by designing the expression cassette to comprise both a sense sequence and an antisense sequence. Alternatively, separate expression cassettes may be used for the sense and antisense sequences. Multiple plant lines transformed with the dsRNA interference expression cassette or expression cassettes are then screened to identify plant lines that show the desired degree of inhibition of polypeptide expression. Methods for using dsRNA interference to inhibit the expression of endogenous plant genes are described in Waterhouse et al., (1998) Proc. Natl. Acad. Sci. USA 95:13959-64, Liu et al., (2002) Plant Physiol. 129:1732-43 and WO 1999/49029, WO 1999/53050, WO 1999/61631 and WO 2000/49035, each of which is herein incorporated by reference.

In certain embodiments, decreasing the expression of the CYP710A target gene is obtained by hairpin RNA (hpRNA) interference or intron-containing hairpin RNA (ihpRNA) interference. These methods are highly efficient at inhibiting the expression of endogenous genes. See, Waterhouse & Helliwell, (2003) Nat. Rev. Genet. 4:29-38, and the references cited therein.

For hpRNA interference, the expression cassette is designed to express an RNA molecule that hybridizes with itself to form a hairpin structure that comprises a single-stranded loop region and a base-paired stem. The base-paired stem region comprises a sense sequence corresponding to all or part of the endogenous messenger RNA encoding the gene whose expression is to be inhibited, and an antisense sequence that is fully or partially complementary to the sense sequence. Alternatively, the base-paired stem region may correspond to a portion of a promoter sequence controlling expression of the gene whose expression is to be inhibited. Thus, the base-paired stem region of the molecule generally determines the specificity of the RNA interference. hpRNA molecules are highly efficient at inhibiting the expression of endogenous genes and the RNA interference they induce is inherited by subsequent generations of plants. See, e.g., Chuang & Meyerowitz, (2000) Proc. Natl. Acad. Sci. USA 97:4985-90; Stoutjesdijk et al., (2002) Plant Physiol. 129:1723-31; and Waterhouse & Helliwell, (2003) Nat. Rev. Genet. 4:29-38. Methods for using hpRNA interference to inhibit or silence the expression of genes are described, e.g., in Chuang & Meyerowitz, (2000) Proc. Natl. Acad. Sci. USA 97:4985-90; Stoutjesdijk et al., (2002) Plant Physiol. 129:1723-31; Waterhouse &d Helliwell, (2003) Nat. Rev. Genet. 4:29-38; Pandolfini et al., BMC Biotechnology 3:7; and US Patent Application Publication No. 2003/0175965, each of which is herein incorporated by reference. A transient assay for the efficiency of hpRNA constructs to silence gene expression in vivo has been described by Panstruga et al., (2003) Mol. Biol. Rep. 30:135-40, herein incorporated by reference.

For ihpRNA, the interfering molecules have the same general structure as for hpRNA, but the RNA molecule additionally comprises an intron that is capable of being spliced in the cell in which the ihpRNA is expressed. The use of an intron minimizes the size of the loop in the hairpin RNA molecule following splicing, and this increases the efficiency of interference. See, e.g., Smith et al., (2000) Nature 407:319-20. In fact, Smith et al., show 100% suppression of endogenous gene expression using ihpRNA-mediated interference. Methods for using ihpRNA interference to inhibit the expression of endogenous plant genes are described, for example, in Smith et al., (2000) Nature 407:319-20; Wesley et al., (2001) Plant J. 27:581-90; Wang & Waterhouse, (2001) Curr. Opin. Plant Biol. 5:146-50; Waterhouse & Helliwell, (2003) Nat. Rev. Genet. 4:29-38; Helliwell & Waterhouse, (2003) Methods 30:289-95; and US Patent Application Publication No. 2003/0180945, each of which is herein incorporated by reference.

Any region of the target polynucleotide can be used to design the domain of the silencing element that shares sufficient sequence identity to allow expression of the hairpin transcript to decrease the level of the target polynucleotide. For instance, the domain can be designed to share sequence identity to the 5′ untranslated region of the target polynucleotide(s), the 3′ untranslated region of the target polynucleotide(s), exonic regions of the target polynucleotide(s), intronic regions of the target polynucleotide(s), and any combination thereof. In specific embodiments, a domain of the silencing element shares sufficient homology to at least about 15, 16, 17, 18, 19, 20, 22, 25 or 30 consecutive nucleotides from about nucleotides 1-50, 25-75, 75-125, 50-100, 125-175, 175-225, 100-150, 150-200, 200-250, 225-275, 275-325, 250-300, 325-375, 375-425, 300-350, 350-400, 425-475, 400-450, 475-525, 450-500, 525-575, 575-625, 550-600, 625-675, 675-725, 600-650, 625-675, 675-725, 650-700, 725-825, 825-875, 750-800, 875-925, 925-975, 850-900, 925-975, 975-1025, 950-1000, 1000-1050, 1025-1075, 1075-1125, 1050-1100, 1125-1175, 1100-1200, 1175-1225, 1225-1275, 1200-1300, 1325-1375, 1375-1425, 1300-1400, 1425-1475, 1475-1525, 1400-1500, 1525-1575, 1575-1625, 1625-1675, 1675-1725, 1725-1775, 1775-1825, 1825-1875, 1875-1925, 1925-1975, 1975-2025, 2025-2075, 2075-2125, 2125-2175, 2175-2225, 1500-1600, 1600-1700, 1700-1800, 1800-1900, 1900-2000 of the target sequence. In some instances, to optimize the siRNA sequences employed in the hairpin, the synthetic oligodeoxyribonucleotide/RNAse H method can be used to determine sites on the target mRNA that are in a conformation that is susceptible to RNA silencing. See, e.g., Vickers et al., (2003) J. Biol. Chem. 278:7108-18 and Yang et al., (2002) Proc. Natl. Acad. Sci. USA 99:9442-47, herein incorporated by reference. These studies indicate that there is a significant correlation between the RNase-H-sensitive sites and sites that promote efficient siRNA-directed mRNA degradation.

The expression cassette for hpRNA interference may also be designed such that the sense sequence and the antisense sequence do not correspond to an endogenous RNA. In this embodiment, the sense and antisense sequence flank a loop sequence that comprises a nucleotide sequence corresponding to all or part of the endogenous messenger RNA of the target gene. Thus, it is the loop region that determines the specificity of the RNA interference. See, e.g., WO 2002/00904; Mette et al., (2000) EMBO J 19:5194-201; Matzke et al., (2001) Curr. Opin. Genet. Devel. 11:221-27; Scheid et al., (2002) Proc. Natl. Acad. Sci. USA 99:13659-62; Aufsaftz et al., (2002) Proc. Natl. Acad. Sci. 99:16499-506; Sijen et al., Curr. Biol. (2001) 11:436-40, herein incorporated by reference.

In addition, transcriptional gene silencing (TGS) may be accomplished through use of a hairpin suppression element where the inverted repeat of the hairpin shares sequence identity with the promoter region of a target polynucleotide to be silenced. See, for example, Aufsatz et al., (2002) Proc. Natl. Acad. Sci. 99:16499-506 and Mette et al., (2000) EMBO J. 19:5194-201.

Amplicon expression cassettes comprise a plant virus-derived sequence that contains all or part of the target gene but generally not all of the genes of the native virus. The viral sequences present in the transcription product of the expression cassette allow the transcription product to direct its own replication. The transcripts produced by the amplicon may be either sense or antisense relative to the target sequence (i.e., the messenger RNA for the polypeptide). Methods of using amplicons to inhibit the expression of endogenous plant genes are described, for example, in Angell and Baulcombe, (1997) EMBO J. 16:3675-3684; Angell and Baulcombe, (1999) Plant 20:357-362; and U.S. Pat. No. 6,646,805, each of which is herein incorporated by reference.

In certain embodiments, the polynucleotide expressed by the expression cassette is a catalytic RNA or has ribozyme activity specific for the messenger RNA of the polypeptide. Thus, the polynucleotide causes the degradation of the endogenous messenger RNA, resulting in decreased expression of the polypeptide. This method is described, e.g., in U.S. Pat. No. 4,987,071, herein incorporated by reference.

In certain embodiments of the disclosure, decreasing the expression of the CYP710A target gene is obtained by RNA interference by expression of a gene encoding a micro RNA (miRNA) or short-interfering RNA (siRNA) (Meister & Tuschl (2004) Nature 431:343-49 and Bonetta et al., (2004) Nature Methods 1:79-86). miRNAs are regulatory agents consisting of about 22 ribonucleotides. miRNAs are highly efficient at inhibiting the expression of endogenous genes. See, e.g., Palatnik et al., (2003) Nature 425:257-63, herein incorporated by reference. The miRNA can be an “artificial miRNA” or “amiRNA” which comprises a miRNA sequence that is synthetically designed to silence a target sequence.

For miRNA interference, the expression cassette is designed to express an RNA molecule that is modeled on an endogenous miRNA gene. For example, the miRNA gene encodes an RNA that forms a hairpin structure containing a 22-nucleotide sequence that is complementary to another endogenous gene (target sequence). In certain embodiments, the 22-nucleotide sequence is selected from a transcript sequence from the target gene (e.g., SnRK1 and/or SnRK2) and contains 22 nucleotides of the target gene in sense orientation and 21 nucleotides of a corresponding antisense sequence that is complementary to the sense sequence. In certain embodiments, in addition to targeting the target gene directly, genes involved in recombination may also be targeted. Accordingly, in certain embodiments, the 22-nucleotide sequence is selected from a transcript sequence from a gene involved in recombination and contains 22 nucleotides of the gene involved in recombination in sense orientation and 21 nucleotides of a corresponding antisense sequence that is complementary to the sense sequence. miRNA molecules are highly efficient at inhibiting the expression of endogenous genes, and the RNA interference they induce is inherited by subsequent generations of plants.

The heterologous polynucleotide being expressed need not form the dsRNA by itself, but can interact with other sequences in the plant cell to allow the formation of the dsRNA. For example, a chimeric polynucleotide that can selectively silence the target polynucleotide can be generated by expressing a chimeric construct comprising the target sequence for a miRNA or siRNA to a sequence corresponding to all or part of the gene or genes to be silenced. In this embodiment, the dsRNA is “formed” when the target for the miRNA or siRNA interacts with the miRNA present in the cell. The resulting dsRNA can then decrease the level of expression of the gene or genes to be silenced. See, for example, US Application Publication 2007-0130653, entitled “Methods and Compositions for Gene Silencing”, herein incorporated by reference. The construct can be designed to have a target for an endogenous miRNA or, alternatively, a target for a heterologous and/or synthetic miRNA can be employed in the construct. If a heterologous and/or synthetic miRNA is employed, it can be introduced into the cell on the same nucleotide construct as the chimeric polynucleotide or on a separate construct. As discussed elsewhere herein, any method can be used to introduce the construct comprising the heterologous miRNA.

### Polypeptide-Based Inhibition of Gene Expression or Protein Activity

In certain embodiments, the polynucleotide encodes a zinc finger protein that binds to a gene encoding a polypeptide, resulting in decreased expression of the gene. In other embodiments, the zinc finger protein binds to a messenger RNA encoding a polypeptide and prevents its translation. Methods of selecting sites for targeting by zinc finger proteins have been described, for example, in U.S. Pat. No. 6,453,242, and methods for using zinc finger proteins to inhibit the expression of genes in plants are described, for example, in US Patent Application Publication No. 2003/0037355, each of which is herein incorporated by reference.

In certain embodiments of the disclosure, the polynucleotide encodes an antibody that binds to at least one polypeptide and decreases the activity of the polypeptide. In certain embodiments, the binding of the antibody results in increased turnover of the antibody complex by cellular quality control mechanisms. The expression of antibodies in plant cells and the inhibition of molecular pathways by expression and binding of antibodies to proteins in plant cells are well known in the art. See, for example, Conrad & Sonnewald, (2003) Nature Biotech. 21:35-36, incorporated herein by reference.

### Increasing Expression or Activity of a CYP710A Gene

Certain embodiments relate to increasing expression of a CYP710A gene in a plant. The term “increased expression” or “overexpression” as used herein means any form of expression that is additional to the original wild-type expression level. The original wild-type expression level might also be zero (absence of expression). Methods for increasing expression of genes or gene products are well documented in the art and include, for example, overexpression driven by appropriate promoters, the use of transcription enhancers or translation enhancers. Isolated nucleic acids which serve as promoter or enhancer elements may be introduced in an appropriate position (typically upstream) of a non-heterologous form of a polynucleotide so as to upregulate expression of a nucleic acid encoding the protein of interest. For example, endogenous promoters may be altered in vivo by mutation, deletion, and/or substitution (see, Kmiec, U.S. Pat. No. 5,565,350; Zarling et al., WO9322443), or isolated promoters may be introduced into a plant cell in the proper orientation and distance from a CYP710A gene so as to control the expression of the gene.

### Expression Constructs

Methods and compositions are provided to modify (e.g., reduce, increase) the level of expression or activity of a CYP710A target gene in the plant (e.g., a plant cell).

CYP710A polynucleotides as described herein can be provided in an expression construct. Expression constructs generally include regulatory elements that are functional in the intended host cell in which the expression construct is to be expressed. Thus, a person of ordinary skill in the art can select regulatory elements for use in bacterial host cells, yeast host cells, plant host cells, insect host cells, mammalian host cells, and human host cells. Regulatory elements include promoters, transcription termination sequences, translation termination sequences, enhancers, and polyadenylation elements. As used herein, the term “expression construct” refers to a combination of nucleic acid sequences that provides for transcription of an operably linked nucleic acid sequence.

An expression construct can comprise a promoter sequence operably linked to a polynucleotide sequence encoding a CYP710A polypeptide as described herein. Promoters can be incorporated into a polynucleotide using standard techniques known in the art. Multiple copies of promoters or multiple promoters can be used in an expression construct as described herein. In some embodiments, a promoter can be positioned about the same distance from the transcription start site in the expression construct as it is from the transcription start site in its natural genetic environment. Some variation in this distance is permitted without substantial decrease in promoter activity. A transcription start site is typically included in the expression construct.

The choice of promoter will vary depending on the temporal and spatial requirements for expression, and also depending on the host cell to be transformed. Thus, for example, expression of the nucleotide sequences can be in any plant and/or plant part, (e.g., in leaves, in stems, in inflorescences, in roots, seeds and/or seedlings, and the like). In many cases, however, expression in multiple tissues is desirable. Although many promoters from dicotyledons have been shown to be operational in monocotyledons and vice versa, a dicotyledonous promoter may be selected for expression in dicotyledons, and a monocotyledonous promoter for expression in monocotyledons. However, there is no restriction to the provenance of selected promoters; it is sufficient that they are operational in driving the expression of the nucleotide sequences in the desired cell.

If the expression construct is to be provided in or introduced into a plant cell, then plant viral promoters, such as, for example, a cauliflower mosaic virus (CaMV) 35S (including the enhanced CaMV 35S promoter (see, for example U.S. Pat. No. 5,106,739)) or a CaMV 19S promoter or a cassava vein mosaic can be used. Other promoters that can be used for expression constructs in plants include, for example, zein promoters including maize zein promoters, prolifera promoter, Ap3 promoter, heat shock promoters, T-DNA 1′- or 2′-promoter of A. tumefaciens, polygalacturonase promoter, chalcone synthase A (CHS-A) promoter from petunia, tobacco PR-1a promoter, ubiquitin promoter, actin promoter, alcA gene promoter, pin2 promoter (Xu et al., 1993), maize Wipl promoter, maize trpA gene promoter (U.S. Pat. No. 5,625,136), maize CDPK gene promoter, and RUBISCO SSU promoter (U.S. Pat. No. 5,034,322) can also be used. Constitutive promoters (such as the CaMV, ubiquitin, actin, or NOS promoter), developmentally-regulated promoters, and inducible promoters (such as those promoters than can be induced by heat, light, hormones, or chemicals) are also contemplated for use with polynucleotide expression constructs described herein. These various types of promoters are known in the art.

Examples of constitutive promoters include, but are not limited to, cestrum virus promoter (cmp) (U.S. Pat. No. 7,166,770), the rice actin 1 promoter (Wang et al. (1992) Mol. Cell. Biol. 12:3399-3406; as well as U.S. Pat. No. 5,641,876), CaMV 35S promoter (Odell et al. (1985) Nature 313:810-812), CaMV 19S promoter (Lawton et al. (1987) Plant Mol. Biol. 9:315-324), nos promoter (Ebert et al. (1987) Proc. Natl. Acad. Sci USA 84:5745-5749), Adh promoter (Walker et al. (1987) Proc. Natl. Acad. Sci. USA 84:6624-6629), sucrose synthase promoter (Yang & Russell (1990) Proc. Natl. Acad. Sci. USA 87:4144-4148), and the ubiquitin promoter. The constitutive promoter derived from ubiquitin accumulates in many cell types. Ubiquitin promoters have been cloned from several plant species for use in transgenic plants, for example, sunflower (Binet et al., 1991. Plant Science 79: 87-94), maize (Christensen et al., 1989. Plant Molec. Biol. 12: 619-632), and arabidopsis (Norris et al. 1993. Plant Molec. Biol. 21:895-906). The maize ubiquitin promoter (UbiP) has been developed in transgenic monocot systems and its sequence and vectors constructed for monocot transformation are disclosed in the patent publication EP 0 342 926. The ubiquitin promoter is suitable for the expression of the nucleotide sequences in transgenic plants, especially monocotyledons. Further, the promoter expression cassettes described by McElroy et al. (Mol. Gen. Genet. 231: 150-160 (1991)) can be easily modified for the expression of the nucleotide sequences and are particularly suitable for use in monocotyledonous hosts.

In some embodiments, tissue specific/tissue preferred promoters can be used. Tissue specific or preferred expression patterns include, but are not limited to, green tissue specific or preferred, root specific or preferred, stem specific or preferred, and flower specific or preferred. Promoters suitable for expression in green tissue include many that regulate genes involved in photosynthesis and many of these have been cloned from both monocotyledons and dicotyledons. Non-limiting examples of tissue-specific promoters include those associated with genes encoding the seed storage proteins (such as (3-conglycinin, cruciferin, napin and phaseolin), zein or oil body proteins (such as oleosin), or proteins involved in fatty acid biosynthesis (including acyl carrier protein, stearoyl-ACP desaturase and fatty acid desaturases (fad 2-1)), and other nucleic acids expressed during embryo development (such as Bce4, see, e.g., Kridl et al. (1991) Seed Sci. Res. 1:209-219; as well as EP Patent No. 255378). Tissue-specific or tissue-preferential promoters useful for the expression of the nucleotide sequences in plants, particularly maize, include but are not limited to those that direct expression in root, pith, leaf or pollen. Such promoters are disclosed, for example, in WO 93/07278, herein incorporated by reference in its entirety. Other non-limiting examples of tissue specific or tissue preferred promoters include the cotton rubisco promoter disclosed in U.S. Pat. No. 6,040,504; the rice sucrose synthase promoter disclosed in U.S. Pat. No. 5,604,121; the root specific promoter described by de Framond (FEBS 290:103-106 (1991); EP 0 452 269 to Ciba-Geigy); the stem specific promoter described in U.S. Pat. No. 5,625,136 (to Ciba-Geigy) and which drives expression of the maize trpA gene; and the cestrum yellow leaf curling virus promoter disclosed in WO 01/73087, all incorporated by reference.

Additional examples of tissue-specific/tissue preferred promoters include, but are not limited to, the root-specific promoters RCc3 (Jeong et al. Plant Physiol. 153:185-197 (2010)) and RB7 (U.S. Pat. No. 5,459,252), the lectin promoter (Lindstrom et al. (1990) Der. Genet. 11:160-167; and Vodkin (1983) Prog. Clin. Biol. Res. 138:87-98), corn alcohol dehydrogenase 1 promoter (Dennis et al. (1984) Nucleic Acids Res. 12:3983-4000), S-adenosyl-L-methionine synthetase (SAMS) (Vander Mijnsbrugge et al. (1996) Plant and Cell Physiology, 37(8):1108-1115), corn light harvesting complex promoter (Bansal et al. (1992) Proc. Natl. Acad. Sci. USA 89:3654-3658), corn heat shock protein promoter (O'Dell et al. (1985) EMBO J. 5:451-458; and Rochester et al. (1986) EMBO J. 5:451-458), pea small subunit RuBP carboxylase promoter (Cashmore, “Nuclear genes encoding the small subunit of ribulose-1,5-bisphosphate carboxylase” pp. 29-39 In: Genetic Engineering of Plants (Hollaender ed., Plenum Press 1983; and Poulsen et al. (1986) Mol. Gen. Genet. 205:193-200), Ti plasmid mannopine synthase promoter (Langridge et al. (1989) Proc. Natl. Acad. Sci. USA 86:3219-3223), Ti plasmid nopaline synthase promoter (Langridge et al. (1989), supra), petunia chalcone isomerase promoter (van Tunen et al. (1988) EMBO J. 7:1257-1263), bean glycine rich protein 1 promoter (Keller et al. (1989) Genes Dev. 3:1639-1646), truncated CaMV 35S promoter (O'Dell et al. (1985) Nature 313:810-812), potato patatin promoter (Wenzler et al. (1989) Plant Mol. Biol. 13:347-354), root cell promoter (Yamamoto et al. (1990) Nucleic Acids Res. 18:7449), maize zein promoter (Kriz et al. (1987) Mol. Gen. Genet. 207:90-98; Langridge et al. (1983) Cell 34:1015-1022; Reina et al. (1990) Nucleic Acids Res. 18:6425; Reina et al. (1990) Nucleic Acids Res. 18:7449; and Wandelt et al. (1989) Nucleic Acids Res. 17:2354), globulin-1 promoter (Belanger et al. (1991) Genetics 129:863-872), α-tubulin cab promoter (Sullivan et al. (1989) Mol. Gen. Genet. 215:431-440), PEPCase promoter (Hudspeth & Grula (1989) Plant Mol. Biol. 12:579-589), R gene complex-associated promoters (Chandler et al. (1989) Plant Cell 1:1175-1183), and chalcone synthase promoters (Franken et al. (1991) EMBO J. 10:2605-2612). In some particular embodiments, the nucleotide sequences are operatively associated with a root-preferred promoter.

Promoters useful for seed-specific expression include the pea vicilin promoter (Czako et al. (1992) Mol. Gen. Genet. 235:33-40; as well as the seed-specific promoters disclosed in U.S. Pat. No. 5,625,136. Useful promoters for expression in mature leaves are those that are switched on at the onset of senescence, such as the SAG promoter from Arabidopsis (Gan et al. (1995) Science 270:1986-1988).

In addition, promoters functional in plastids can be used. Non-limiting examples of such promoters include the bacteriophage T3 gene 9 5′ UTR and other promoters disclosed in U.S. Pat. No. 7,579,516. Other promoters include but are not limited to the S-E9 small subunit RuBP carboxylase promoter and the Kunitz trypsin inhibitor gene promoter (Kti3).

In some embodiments, inducible promoters can be used. Thus, for example, chemical-regulated promoters can be used to modulate the expression of a gene in a plant through the application of an exogenous chemical regulator. Regulation of the expression of nucleotide sequences via promoters that are chemically regulated enables the polypeptides to be synthesized only when the crop plants are treated with the inducing chemicals. Depending upon the objective, the promoter may be a chemical-inducible promoter, where application of a chemical induces gene expression, or a chemical-repressible promoter, where application of the chemical represses gene expression.

Chemical inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid (e.g., the PR1a system), steroid steroid-responsive promoters (see, e.g., the glucocorticoid-inducible promoter in Schena et al. (1991) Proc. Natl. Acad. Sci. USA 88, 10421-10425 and McNellis et al. (1998) Plant J. 14, 247-257) and tetracycline-inducible and tetracycline-repressible promoters (see, e.g., Gatz et al. (1991) Mol. Gen. Genet. 227, 229-237, and U.S. Pat. Nos. 5,814,618 and 5,789,156, Lac repressor system promoters, copper-inducible system promoters, salicylate-inducible system promoters (e.g., the PR1a system), glucocorticoid-inducible promoters (Aoyama et al. (1997) Plant J. 11:605-612), and ecdysone-inducible system promoters.

Other non-limiting examples of inducible promoters include ABA- and turgor-inducible promoters, the auxin-binding protein gene promoter (Schwob et al. (1993) Plant J. 4:423-432), the UDP glucose flavonoid glycosyl-transferase promoter (Ralston et al. (1988) Genetics 119:185-197), the MPI proteinase inhibitor promoter (Cordero et al. (1994) Plant J. 6:141-150), and the glyceraldehyde-3-phosphate dehydrogenase promoter (Kohler et al. (1995) Plant Mol. Biol. 29:1293-1298; Martinez et al. (1989) J. Mol. Biol. 208:551-565; and Quigley et al. (1989) J. Mol. Evol. 29:412-421). Also included are the benzene sulphonamide-inducible (U.S. Pat. No. 5,364,780) and alcohol-inducible (Int'l Patent Application Publication Nos. WO 97/06269 and WO 97/06268) systems and glutathione 5-transferase promoters. Likewise, one can use any of the inducible promoters described in Gatz (1996) Current Opinion Biotechnol. 7:168-172 and Gatz (1997) Annu. Rev. Plant Physiol. Plant Mol. Biol. 48:89-108. Other chemically inducible promoters useful for directing the expression of the nucleotide sequences in plants are disclosed in U.S. Pat. No. 5,614,395 herein incorporated by reference in its entirety. Chemical induction of gene expression is also detailed in the published application EP 0 332 104 (to Ciba-Geigy) and U.S. Pat. No. 5,614,395. In some embodiments, a promoter for chemical induction can be the tobacco PR-1a promoter.

A number of non-translated leader sequences derived from viruses are known to enhance gene expression. Specifically, leader sequences from Tobacco Mosaic Virus (TMV, the “ω-sequence”), Maize Chlorotic Mottle Virus (MCMV) and Alfalfa Mosaic Virus (AMV) have been shown to be effective in enhancing expression (Gallie et al. (1987) Nucleic Acids Res. 15:8693-8711; and Skuzeski et al. (1990) Plant Mol. Biol. 15:65-79). Other leader sequences known in the art include, but are not limited to, picornavirus leaders such as an encephalomyocarditis (EMCV) 5′ noncoding region leader (Elroy-Stein et al. (1989) Proc. Natl. Acad. Sci. USA 86:6126-6130); potyvirus leaders such as a Tobacco Etch Virus (TEV) leader (Allison et al. (1986) Virology 154:9-20); Maize Dwarf Mosaic Virus (MDMV) leader (Allison et al. (1986), supra); human immunoglobulin heavy-chain binding protein (BiP) leader (Macejak & Samow (1991) Nature 353:90-94); untranslated leader from the coat protein mRNA of AMV (AMV RNA 4; Jobling & Gehrke (1987) Nature 325:622-625); tobacco mosaic TMV leader (Gallie et al. (1989) Molecular Biology of RNA 237-256); and MCMV leader (Lommel et al. (1991) Virology 81:382-385). See also, Della-Cioppa et al. (1987) Plant Physiol. 84:965-968.

Expression constructs may optionally contain a transcription termination sequence, a translation termination sequence, a sequence encoding a signal peptide, and/or enhancer elements. Transcription termination regions can typically be obtained from the 3′ untranslated region of a eukaryotic or viral gene sequence. Transcription termination sequences can be positioned downstream of a coding sequence to provide for efficient termination. A signal peptide sequence is a short amino acid sequence typically present at the amino terminus of a protein that is responsible for the relocation of an operably linked mature polypeptide to a wide range of post-translational cellular destinations, ranging from a specific organelle compartment to sites of protein action and the extracellular environment. Targeting gene products to an intended cellular and/or extracellular destination through the use of an operably linked signal peptide sequence is contemplated for use with the polypeptides described herein. Classical enhancers are cis-acting elements that increase gene transcription and can also be included in the expression construct. Classical enhancer elements are known in the art, and include, but are not limited to, the CaMV 35S enhancer element, cytomegalovirus (CMV) early promoter enhancer element, and the SV40 enhancer element. Intron-mediated enhancer elements that enhance gene expression are also known in the art. These elements must be present within the transcribed region and are orientation dependent. Examples include the maize shrunken-1 enhancer element (Clancy and Hannah, 2002).

An expression construct can include a nucleotide sequence for a selectable marker, which can be used to select a transformed plant, plant part and/or plant cell. As used herein, “selectable marker” means a nucleotide sequence that when expressed imparts a distinct phenotype to the plant, plant part and/or plant cell expressing the marker and thus allows such transformed plants, plant parts and/or plant cells to be distinguished from those that do not have the marker. Such a nucleotide sequence may encode either a selectable or screenable marker, depending on whether the marker confers a trait that can be selected for by chemical means, such as by using a selective agent (e.g., an antibiotic, herbicide, or the like), or on whether the marker is simply a trait that one can identify through observation or testing, such as by screening. Of course, many examples of suitable selectable markers are known in the art and can be used in the expression cassettes described herein.

Examples of selectable markers include, but are not limited to, a nucleotide sequence encoding neo or nptII, which confers resistance to kanamycin, G418, and the like (Potrykus et al. (1985) Mol. Gen. Genet. 199:183-188); a nucleotide sequence encoding bar, which confers resistance to phosphinothricin; a nucleotide sequence encoding an altered 5-enolpyruvylshikimate-3-phosphate (EPSP) synthase, which confers resistance to glyphosate (Hinchee et al. (1988) Biotech. 6:915-922); a nucleotide sequence encoding a nitrilase such as bxn from Klebsiella ozaenae that confers resistance to bromoxynil (Stalker et al. (1988) Science 242:419-423); a nucleotide sequence encoding an altered acetolactate synthase (ALS) that confers resistance to imidazolinone, sulfonylurea or other ALS-inhibiting chemicals (EP Patent Application No. 154204); a nucleotide sequence encoding a methotrexate-resistant dihydrofolate reductase (DHFR) (Thillet et al. (1988) J. Biol. Chem. 263:12500-12508); a nucleotide sequence encoding a dalapon dehalogenase that confers resistance to dalapon; a nucleotide sequence encoding a mannose-6-phosphate isomerase (also referred to as phosphomannose isomerase (PMI)) that confers an ability to metabolize mannose (U.S. Pat. Nos. 5,767,378 and 5,994,629); a nucleotide sequence encoding an altered anthranilate synthase that confers resistance to 5-methyl tryptophan; and/or a nucleotide sequence encoding hph that confers resistance to hygromycin. One of skill in the art is capable of choosing a suitable selectable marker for use in an expression cassette.

Additional selectable markers include, but are not limited to, a nucleotide sequence encoding β-glucuronidase or uidA (GUS) that encodes an enzyme for which various chromogenic substrates are known; an R-locus nucleotide sequence that encodes a product that regulates the production of anthocyanin pigments (red color) in plant tissues (Dellaporta et al., “Molecular cloning of the maize R-nj allele by transposon-tagging with Ac,” pp. 263-282 In: Chromosome Structure and Function: Impact of New Concepts, 18th Stadler Genetics Symposium (Gustafson & Appels eds., Plenum Press 1988)); a nucleotide sequence encoding β-lactamase, an enzyme for which various chromogenic substrates are known (e.g., PADAC, a chromogenic cephalosporin) (Sutcliffe (1978) Proc. Natl. Acad. Sci. USA 75:3737-3741); a nucleotide sequence encoding xylE that encodes a catechol dioxygenase (Zukowsky et al. (1983) Proc. Natl. Acad. Sci. USA 80:1101-1105); a nucleotide sequence encoding tyrosinase, an enzyme capable of oxidizing tyrosine to DOPA and dopaquinone, which in turn condenses to form melanin (Katz et al. (1983) J. Gen. Microbiol. 129:2703-2714); a nucleotide sequence encoding β-galactosidase, an enzyme for which there are chromogenic substrates; a nucleotide sequence encoding luciferase (lux) that allows for bioluminescence detection (Ow et al. (1986) Science 234:856-859); a nucleotide sequence encoding aequorin, which may be employed in calcium-sensitive bioluminescence detection (Prasher et al. (1985) Biochem. Biophys. Res. Comm. 126:1259-1268); or a nucleotide sequence encoding green fluorescent protein (Niedz et al. (1995) Plant Cell Reports 14:403-406). One of skill in the art is capable of choosing a suitable selectable marker for use in an expression cassette.

Optionally the gene encoding the CYP710A polynucleotide is codon optimized to remove features inimical to expression and codon usage is optimized for expression in the particular crop (see, for example, U.S. Pat. No. 6,051,760; EP 0359472; EP 80385962; EP 0431829; and Perlak et al. (1991) PNAS USA 88:3324-3328; all of which are herein incorporated by reference).

It is further recognized that various expression constructs other than CYP710A polynucleotide constructs are described herein. For example, expression constructs encoding an RNA-guided endonuclease or other genome editing molecules are described herein. In certain embodiments, a plant cell is transformed with a DNA construct or expression cassette for expression of at least one silencing element. Such methods and compositions can employ an expression construct comprising an element that when expressed reduces target gene polynucleotide and/or target gene polypeptide expression level or activity and is operably linked to a promoter functional in a plant cell. One of skill will understand how to apply the above disclosure to any expression construct.

### Transformation Methods

Several embodiments relate to plant cells, plant tissues, plants, and seeds that comprise a recombinant DNA (e.g., a CYP710A polynucleotide, a silencing element, a genome editing molecule) as described herein.

Suitable methods for transformation of host plant cells include virtually any method by which DNA or RNA can be introduced into a cell (for example, where a recombinant DNA construct is stably integrated into a plant chromosome or where a recombinant DNA construct or an RNA is transiently provided to a plant cell) and are well known in the art. Two effective methods for cell transformation are Agrobacterium-mediated transformation and microprojectile bombardment-mediated transformation. Microprojectile bombardment methods are illustrated, for example, in U.S. Pat. Nos. 5,550,318; 5,538,880; 6,160,208; and 6,399,861. Agrobacterium-mediated transformation methods are described, for example in U.S. Pat. No. 5,591,616, which is incorporated herein by reference in its entirety. Transformation of plant material is practiced in tissue culture on nutrient media, for example a mixture of nutrients that allow cells to grow in vitro. Recipient cell targets include, but are not limited to, meristem cells, shoot tips, hypocotyls, calli, immature or mature embryos, and gametic cells such as microspores and pollen. Callus can be initiated from tissue sources including, but not limited to, immature or mature embryos, hypocotyls, seedling apical meristems, microspores and the like. Cells containing a transgenic nucleus are grown into transgenic plants.

In transformation, DNA is typically introduced into only a small percentage of target plant cells in any one transformation experiment. Marker genes are used to provide an efficient system for identification of those cells that are stably transformed by receiving and integrating a recombinant DNA molecule into their genomes. Preferred marker genes provide selective markers which confer resistance to a selective agent, such as an antibiotic or an herbicide. Potentially transformed cells are exposed to the selective agent. In the population of surviving cells are those cells where, generally, the resistance-conferring gene is integrated and expressed at sufficient levels to permit cell survival. Cells can be tested further to confirm stable integration of the exogenous DNA. Commonly used selective marker genes include those conferring resistance to antibiotics such as kanamycin and paromomycin (nptII), hygromycin B (aph IV), spectinomycin (aadA) and gentamycin (aac3 and aacC4) or resistance to herbicides such as glufosinate (bar or pat), dicamba (DMO) and glyphosate (aroA or EPSPS). Examples of such selectable markers are illustrated in U.S. Pat. Nos. 5,550,318; 5,633,435; 5,780,708 and 6,118,047. Markers which provide an ability to visually screen transformants can also be employed, for example, a gene expressing a colored or fluorescent protein such as a luciferase or green fluorescent protein (GFP) or a gene expressing a beta-glucuronidase or uidA gene (GUS) for which various chromogenic substrates are known.

Transformation of a cell may be stable or transient. Thus, in certain embodiments, a plant cell is stably transformed with a nucleic acid molecule. In other embodiments, a plant is transiently transformed with a nucleic acid molecule. “Transient transformation” in the context of a polynucleotide means that a polynucleotide is introduced into the cell and does not integrate into the genome of the cell. By “stably introducing” or “stably introduced” in the context of a polynucleotide introduced into a cell is intended the introduced polynucleotide is stably incorporated into the genome of the cell, and thus the cell is stably transformed with the polynucleotide.

“Stable transformation” or “stably transformed” as used herein means that a nucleic acid is introduced into a cell and integrates into the genome of the cell. As such, the integrated nucleic acid is capable of being inherited by the progeny thereof, more particularly, by the progeny of multiple successive generations. “Genome” as used herein also includes the nuclear and the plastid genome, and therefore includes integration of the nucleic acid into, for example, the chloroplast genome. Stable transformation as used herein can also refer to a transgene that is maintained extrachromasomally, for example, as a minichromosome.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a plant). Stable transformation of a cell can be detected by, for example, a Northern blot hybridization assay of RNA of the cell with nucleic acid sequences which specifically hybridize with a nucleotide sequence of a transgene introduced into a plant or other organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods Transformation can also be detected by direct sequencing and/or hybridization protocols well known in the art.

In certain embodiments, transformation of a cell comprises nuclear transformation. In other embodiments, transformation of a cell comprises plastid transformation (e.g., chloroplast transformation).

Procedures for transforming plants are well known and routine in the art and are described throughout the literature. Non-limiting examples of methods for transformation of plants include transformation via bacterial-mediated nucleic acid delivery (e.g., via Agrobacteria), viral-mediated nucleic acid delivery, silicon carbide or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) and/or biological mechanism that results in the introduction of nucleic acid into the plant cell, including any combination thereof. General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)).

Agrobacterium-mediated transformation is a commonly used method for transforming plants, in particular, dicot plants, because of its high efficiency of transformation and because of its broad utility with many different species. Agrobacterium-mediated transformation typically involves transfer of the binary vector carrying the foreign DNA of interest to an appropriate Agrobacterium strain that may depend on the complement of vir genes carried by the host Agrobacterium strain either on a co-resident Ti plasmid or chromosomally (Uknes et al. (1993) Plant Cell 5:159-169). The transfer of the recombinant binary vector to Agrobacterium can be accomplished by a triparental mating procedure using Escherichia coli carrying the recombinant binary vector, a helper E. coli strain that carries a plasmid that is able to mobilize the recombinant binary vector to the target Agrobacterium strain. Alternatively, the recombinant binary vector can be transferred to Agrobacterium by nucleic acid transformation (Hagen & Willmitzer (1988) Nucleic Acids Res. 16:9877).

Transformation of a plant by recombinant Agrobacterium usually involves co-cultivation of the Agrobacterium with explants from the plant and follows methods well known in the art. Transformed tissue is regenerated on selection medium carrying an antibiotic or herbicide resistance marker between the binary plasmid T-DNA borders.

Another method for transforming plants, plant parts and/or plant cells involves propelling inert or biologically active particles at plant tissues and cells. See, e.g., U.S. Pat. Nos. 4,945,050; 5,036,006 and 5,100,792. Generally, this method involves propelling inert or biologically active particles at the plant cells under conditions effective to penetrate the outer surface of the cell and afford incorporation within the interior thereof. When inert particles are utilized, the vector can be introduced into the cell by coating the particles with the vector containing the nucleic acid of interest. Alternatively, a cell or cells can be surrounded by the vector so that the vector is carried into the cell by the wake of the particle. Biologically active particles (e.g., dried yeast cells, dried bacterium or a bacteriophage, each containing one or more nucleic acids sought to be introduced) also can be propelled into plant tissue.

Thus, in particular embodiments, a plant cell can be transformed by any method known in the art and as described herein and intact plants can be regenerated from these transformed cells using any of a variety of known techniques. Plant regeneration from plant cells, plant tissue culture and/or cultured protoplasts is described, for example, in Evans et al. (Handbook of Plant Cell Cultures, Vol. 1, MacMilan Publishing Co. New York (1983)); and Vasil I. R. (ed.) (Cell Culture and Somatic Cell Genetics of Plants, Acad. Press, Orlando, Vol. I (1984), and Vol. II (1986)). Methods of selecting for transformed transgenic plants, plant cells and/or plant tissue culture are routine in the art and can be employed in the methods provided herein.

Likewise, the genetic properties engineered into the transgenic seeds and plants, plant parts, and/or plant cells described above can be passed on by sexual reproduction or vegetative growth and therefore can be maintained and propagated in progeny plants. Generally, maintenance and propagation make use of known agricultural methods developed to fit specific purposes such as harvesting, sowing or tilling.

A nucleotide sequence therefore can be introduced into the plant, plant part and/or plant cell in any number of ways that are well known in the art. The methods do not depend on a particular method for introducing one or more nucleotide sequences into a plant, only that they gain access to the interior of at least one cell of the plant. Where more than one nucleotide sequence is to be introduced, they can be assembled as part of a single nucleic acid construct, or as separate nucleic acid constructs, and can be located on the same or different nucleic acid constructs. Accordingly, the nucleotide sequences can be introduced into the cell of interest in a single transformation event, in separate transformation events, or, for example, in plants, as part of a breeding protocol.

Plants with Increased Sitosterol or Hard Endosperm

Several embodiments relate to plant cells, plant tissues, plants, and seeds that comprise reduced expression or activity an endogenous CYP710A gene. Plants may be monocots or dicots, and may include, for example, rice, wheat, barley, oats, rye, sorghum, maize, grape, tomato, potato, lettuce, broccoli, cucumber, peanut, melon, pepper, carrot, squash, onion, soybean, alfalfa, sunflower, cotton, canola, and sugar beet plants.

Plants that are useful in the methods of the present disclosure include all plants which belong to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amaranth, artichoke, asparagus, broccoli, Brussels sprouts, cabbage, canola, carrot, cauliflower, celery, collard greens, flax, kale, lentil, oilseed rape, okra, onion, potato, rice, soybean, strawberry, sugar beet, sugar cane, sunflower, tomato, squash, tea and algae, amongst others. In certain embodiments, the plant is a crop plant. Examples of crop plants include inter alia soybean, sunflower, canola, alfalfa, rapeseed, cotton, tomato, potato, or tobacco. In certain embodiments, the plant is a maize plant.

Certain embodiments encompass a progeny or a descendant of a plant with increased sitosterol or increased hard endosperm as well as seeds derived from the plants with increased sitosterol or hard endosperm and cells derived from the plants with increased sitosterol or hard endosperm as described herein.

In certain embodiments, plant cells of the present disclosure are capable of regenerating a plant or plant part. In other embodiments, plant cells are not capable of regenerating a plant or plant part. Examples of cells not capable of regenerating a plant include, but are not limited to, endosperm, seed coat (testa and pericarp), and root cap.

Besides seed, elevated levels of sterols, such as sitosterol, can be found in other parts of the plants encompassed herein. For example, elevated levels of sterols, phytosterols, etc., can be expected in fruits, as well as vegetable parts of plants other than seeds. Vegetable parts of plants include, for example, pollen, inflorescences, terminal buds, lateral buds, stems, leaves, tubers, and roots. Thus, the present disclosure also encompasses these and other parts of the plants disclosed herein that contain elevated levels of desirable phytosterol.

Several embodiments provide a commodity plant product prepared from the plants with increased sitosterol or increased hard endosperm. In certain embodiments, examples of plant products include, without limitation, grain, oil, and meal. In one embodiment, a commodity plant product is plant grain (e.g., grain suitable for use as feed or for processing), plant oil (e.g., oil suitable for use as food or biodiesel), or plant meal (e.g., meal suitable for use as feed). A preferred commodity plant product is fodder, seed meal, oil, or seed-treatment-coated seeds.

Of course, a significant effect of reducing expression or activity of an endogenous CYP710A gene disclosed herein will be on the content of phytosterols of seed oil. Therefore, additional aspects of the present disclosure include oil obtainable from the seed of the plants described herein, and methods for producing such plants and oil. Methods for extracting and processing seed oils are well known in the art. Oils produced by the cells, plants, and methods disclosed herein are superior in phytosterol composition to conventional oils in a variety of ways. For example, oil of the present disclosure can contain an elevated level of sitosterol.

The product may be produced at the site where the plant has been grown, the plants and/or parts thereof may be removed from the site where the plants have been grown to produce the product. Typically, the plant is grown, the desired harvestable parts are removed from the plant, if feasible in repeated cycles, and the product made from the harvestable parts of the plant. The step of growing the plant may be performed only once each time the method is performed, while allowing repeated times the steps of product production e.g. by repeated removal of harvestable parts of the plants of the disclosure and if necessary further processing of these parts to arrive at the product. It is also possible that the step of growing the plants is repeated and plants or harvestable parts are stored until the production of the product is then performed once for the accumulated plants or plant parts. Also, the steps of growing the plants and producing the product may be performed with an overlap in time, even simultaneously to a large extend or sequentially. Generally, the plants are grown for some time before the product is produced.

The plants of the disclosure may be used in a plant breeding program. The goal of plant breeding is to combine, in a single variety or hybrid, various desirable traits. For field crops, these traits may include, for example, resistance to diseases and insects, tolerance to heat and drought, tolerance to chilling or freezing, reduced time to crop maturity, greater yield and better agronomic quality. With mechanical harvesting of many crops, uniformity of plant characteristics such as germination and stand establishment, growth rate, maturity, and plant height is desirable. Traditional plant breeding is an important tool in developing new and improved commercial crops. This disclosure encompasses methods for producing a plant by crossing a first parent plant with a second parent plant wherein one or both of the parent plants is a plant displaying a phenotype as described herein.

Plant breeding techniques known in the art and used in a plant breeding program include, but are not limited to, recurrent selection, bulk selection, mass selection, backcrossing, pedigree breeding, open pollination breeding, restriction fragment length polymorphism enhanced selection, genetic marker enhanced selection, doubled haploids and transformation. Often combinations of these techniques are used.

The development of hybrids in a plant breeding program requires, in general, the development of homozygous inbred lines, the crossing of these lines and the evaluation of the crosses. There are many analytical methods available to evaluate the result of a cross. The oldest and most traditional method of analysis is the observation of phenotypic traits. Alternatively, the genotype of a plant can be examined.

A genetic trait which has been engineered into a particular plant using transformation techniques can be moved into another line using traditional breeding techniques that are well known in the plant breeding arts. For example, a backcrossing approach is commonly used to move a transgene from a transformed plant to an elite inbred line and the resulting progeny would then comprise the transgene(s). Also, if an inbred line was used for the transformation, then the transgenic plants could be crossed to a different inbred in order to produce a transgenic hybrid plant. As used herein, “crossing” can refer to a simple X by Y cross or the process of backcrossing, depending on the context.

The development of a hybrid in a plant breeding program involves three steps: (1) the selection of plants from various germplasm pools for initial breeding crosses; (2) the selfing of the selected plants from the breeding crosses for several generations to produce a series of inbred lines, which, while different from each other, breed true and are highly homozygous and (3) crossing the selected inbred lines with different inbred lines to produce the hybrids. During the inbreeding process, the vigor of the lines decreases. Vigor is restored when two different inbred lines are crossed to produce the hybrid. An important consequence of the homozygosity and homogeneity of the inbred lines is that the hybrid created by crossing a defined pair of inbreds will always be the same. Once the inbreds that give a superior hybrid have been identified, the hybrid seed can be reproduced indefinitely as long as the homogeneity of the inbred parents is maintained.

Plants of the present disclosure may be used to produce, e.g., a single cross hybrid, a three-way hybrid or a double cross hybrid. A single cross hybrid is produced when two inbred lines are crossed to produce the F1 progeny. A double cross hybrid is produced from four inbred lines crossed in pairs (A×B and C×D) and then the two F1 hybrids are crossed again (A×B) times (C×D). A three-way cross hybrid is produced from three inbred lines where two of the inbred lines are crossed (A×B) and then the resulting F1 hybrid is crossed with the third inbred (A×B)×C. Much of the hybrid vigor and uniformity exhibited by F1 hybrids is lost in the next generation (F2). Consequently, seed produced by hybrids is consumed rather than planted.

### Embodiments

The following numbered embodiments also form part of the present disclosure:

1. A modified plant, or a progeny, a plant part, or a plant cell thereof, with increased sitosterol or hard endosperm, the plant comprising reduced expression or activity of an endogenous CYP710A gene relative to a corresponding unmodified plant.

2. The modified plant of claim 1, wherein the endogenous CYP710A gene encodes a polypeptide comprising an amino acid sequence having at least 80%, at least 90%, at least 95%, at least 99%, or 100% sequence identity to SEQ ID NO: 2.

3. The modified plant of claim 1 or claim 2, wherein the endogenous CYP710A gene comprises a nucleotide sequence having at least 80%, at least 90%, at least 95%, at least 99%, or 100% sequence identity to SEQ ID NO: 1.

4. The modified plant of any one of claims 1-3, wherein the modified plant comprises one or more nucleotide modifications at a genomic locus comprising the CYP710A gene.

5. The modified plant of any one of claims 1-3, wherein the one or more nucleotide modifications are present within the coding region, non-coding region, regulatory sequence, or untranslated region of the endogenous CYP710A gene.

6. The modified plant of any one of claims 1-5, wherein the one or more nucleotide modifications reduce expression of the CYP710A gene, reduce transcriptional activity of the polypeptide encoded by the CYP710A gene, generate one or more alternative spliced variants of the CYP710A gene, introduce a frameshift mutation in one or more exons of the CYP710A gene, delete a substantial portion of the CYP710A gene, delete a full-length open reading frame of the CYP710A gene, repress an enhancer motif present within a regulatory region encoding the CYP710A gene, or modify one or more nucleotides of a regulatory element operably linked to the CYP710A gene, or any combination thereof.

7. The modified plant of any one of claims 1-6, wherein the modified plant comprises a silencing element that targets the endogenous CYP710A gene.

8. The modified plant of any one of claims 1-7, wherein the silencing element comprises a double stranded RNA, a siRNA, a miRNA, or a hairpin suppression element.

9. The modified plant of any one of claims 1-8, wherein the plant is a maize plant.

10. The modified plant of any one of claims 1-9, wherein the plant comprises a nucleotide sequence having at least 80%, at least 90%, at least 95%, at least 99%, or 100% sequence identity to SEQ ID NO: 3.

11. The modified plant of any one of claims 1-10, wherein the sitosterol is increased at least 1.2-fold, at least 1.3-fold, at least 1.4-fold, or at least 1.5-fold in the seed of the plant relative to a corresponding unmodified plant.

12. A seed or an asexual propagate of the modified plant of any one of claims 1-11.

13. A method for producing a plant with increased sitosterol or hard endosperm, the method comprising: reducing expression or activity of an endogenous CYP710A gene in the plant.

14. The method of claim 13, wherein the endogenous CYP710A gene encodes a polypeptide comprising an amino acid sequence having at least 80%, at least 90%, at least 95%, at least 99%, or 100% sequence identity to SEQ ID NO: 2.

15. The method of claim 13 or claim 14, wherein the endogenous CYP710A gene comprises a nucleotide sequence having at least 80%, at least 90%, at least 95%, at least 99%, or 100% sequence identity to SEQ ID NO: 1.

16. The method of any one of claims 13-15, wherein the expression or activity is reduced by introducing one or more nucleotide modifications at a genomic locus comprising the CYP710A gene.

17. The method of any one of claims 13-16, wherein the one or more nucleotide modifications are introduced through targeted DNA modification.

18. The method of any one of claims 13-17, wherein the one or more nucleotide modifications are introduced through use of a guide RNA and an RNA-guided endonuclease.

19. The method of any one of claims 13-18, wherein the one or more nucleotide modifications are introduced within the coding region, non-coding region, regulatory sequence, or untranslated region of the endogenous CYP710A gene.

20. The method of any one of claims 13-19, wherein the one or more nucleotide modifications reduce expression of the CYP710A gene, reduce transcriptional activity of the polypeptide encoded by the CYP710A gene, generate one or more alternative spliced variants of the CYP710A gene, introduce a frameshift mutation in one or more exons of the CYP710A gene, delete a substantial portion of the CYP710A gene, delete a full-length open reading frame of the CYP710A gene, repress an enhancer motif present within a regulatory region encoding the CYP710A gene, or modify one or more nucleotides of a regulatory element operably linked to the CYP710A gene, or any combination thereof.

21, The method of any one of claims 13-20, wherein the expression or activity is reduced by introducing a silencing element that targets the endogenous CYP710A gene.

22. The method of any one of claims 13-21, wherein the silencing element comprises a double stranded RNA, a siRNA, a miRNA, or a hairpin suppression element.

23. The method of any one of claims 13-22, wherein the plant is a maize plant.

24. The method of any one of claims 13-23, wherein the sitosterol is increased at least 1.2-fold, at least 1.3-fold, at least 1.4-fold, or at least 1.5-fold in the seed of the plant relative to a corresponding unmodified plant.

25. A method of producing a plant with increased sitosterol or hard endosperm, the method comprising: (a) crossing the plant of any one of claims 1-11 with itself or another plant to produce seed; and (b) growing a progeny plant from the seed to produce a plant with increased sitosterol or hard endosperm.

26. The method of claim 25, further comprising: (c) crossing the progeny plant with itself or another plant; and (d) repeating steps (b) and (c) for an additional 0-7 generations to produce a plant having increased sitosterol or hard endosperm.

27. A crop comprising a plurality of the plants of any one of claims 1-11 planted together in an agricultural field.

28. A method of producing seeds having increased sitosterol or hard endosperm from a plant crop, the method comprising: cultivating a plurality of the plants of any one of claims 1-11 as a plant crop, and harvesting seeds from the plant crop, wherein the seeds have increased sitosterol or hard endosperm relative to a corresponding unmodified plant.

29. A commodity plant product prepared from the plant, plant part, or plant cell of any one of claims 1-11, wherein the commodity plant product comprises a nucleic acid comprising one or more nucleotide modifications at a genomic locus comprising the CYP710A gene.

30. The commodity plant product of claim 29, wherein the product is fodder, seed meal, oil, or seed-treatment-coated seed.

31. A method for producing a commodity plant product, the method comprising processing the plant or plant part of any one of claims 1-11 to obtain the product.

32. The method of claim 31, wherein the commodity plant product is fodder, seed meal, oil, or seed-treatment-coated seeds

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

Although the foregoing disclosure has been described in some detail by way of illustration and example for purposes of clarity of understanding, it will be obvious that certain changes and modifications may be practiced within the scope of the appended claims.

The following examples are offered by way of illustration and not by way of limitation.

### EXAMPLES

### Example 1: Identification of a Maize Sterol C-22 Desaturase Involved in Stigmasterol Biosynthesis

Sterols are important components of plasma membranes in eukaryotic organisms. They are also precursors for synthesis of steroid hormones such as testosterone, estrogen, glucocorticoids and mineral corticoids in mammals, ecdysteroids in insects and crustaceans, antheridiol and oogoniol (mating hormones of fungi), and BR in plants. Cholesterol is the major sterol in vertebrates, while fungi and some unicellular algae synthesize ergosterol as their principal sterol. In contrast, plants produce a mixture of sterols including cholesterol, sitosterol, stigmasterol, and campesterol, with sitosterol being the most abundant.

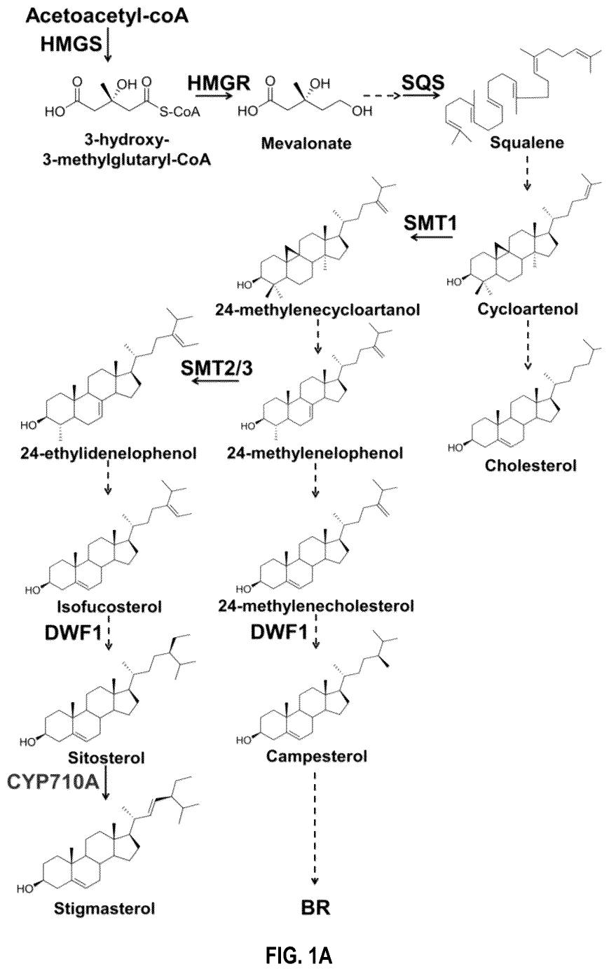

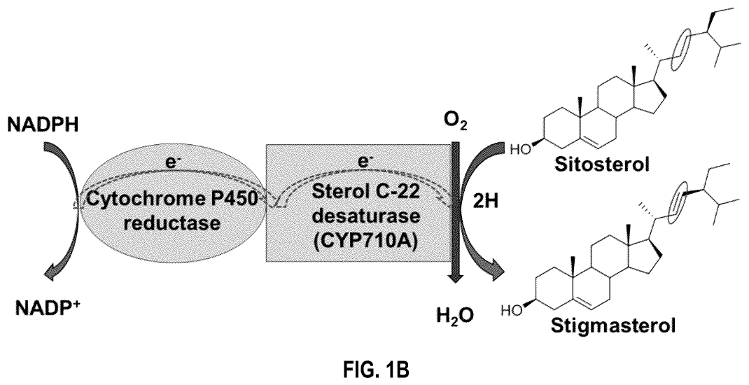

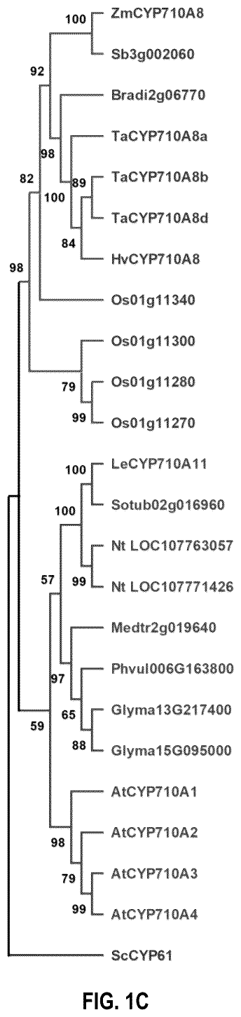

The biosynthesis of plant sterols occurs primarily through the mevalonate (MVA) pathway (FIG. 1A) and involves several enzyme-catalyzed steps. Key enzymes in the MVA pathway including HMGS, HMGR, and SQS catalyze cytosolic reactions leading to the formation of squalene. Reactions beyond squalene synthesis occur in the ER producing cycloartenol in plants or lanosterol in vertebrates and yeast. Cycloartenol is a target of SMT1 to direct carbon flow towards the plant sterol (phytosterol) pathway. Consequently, the activities of SMT1 and SMT2/3 result in methylated sterols (campesterol and crinosterol) and ethylated sterols (sitosterol and stigmasterol), respectively. Cycloartenol is also used by sterol side chain reductase (SSR) to synthesize cholesterol. Downstream of SMT1 and SMT2/3, DWF1 contributes to both the stigmasterol and campesterol branches, and campesterol is used to produce crinosterol and BR. Sitosterol can be transformed to stigmasterol via a reaction catalyzed by cytochrome P450 CYP710A sterol C-22 desaturase.

Identification of putative sterol C-22 desaturases in maize involved querying the MaizeGDB database (maizegdb.org) with AtCYP710A1 amino acid sequence. The query resulted in a single 1,551 bp intron-less gene model (Zm00001d039384) predicted to encode a 515 aa protein with 63% similarity to AtCYP710A1, which was renamed to ZmCYP710A8 as per P450 nomenclature. Multiple sequence alignment showed that the amino acid sequence of ZmCYP710A8 is highly similar to CYP61 from yeast, and previously characterized CYP710A proteins from plants. Further, conserved sites and characteristic motifs for CYP710A are also present in ZmCYP710A8.

Phylogenetic analysis suggested that other grass species may also encode single sterol C-22 desaturases (FIG. 1C). The CYP710A-like homologues cluster into two monophyletic clades, monocots, and eudicots, with ZmCYP710A8 placed in the monocot Glade. Moreover, no CYP710A-like gene from either Glade clustered together, suggesting that the plant CYP710A-like genes are from a single ancestor and developed into different branches after the lineages diverged. Within the clades, some species have more than one representative, suggesting these paralogs might have resulted from recent genome duplication events. These findings suggested that ZmCYP710A8 may encode the maize sterol C-22 desaturase.

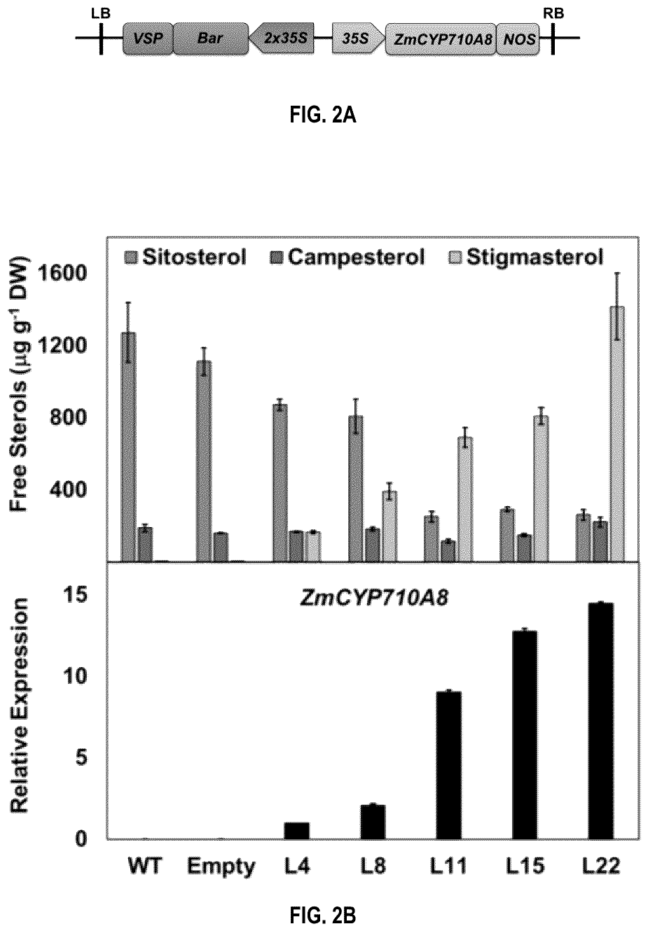

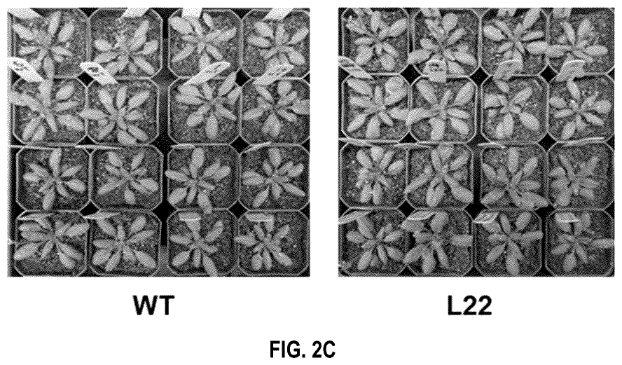

To validate the function of ZmCYP710A8 in stigmasterol biosynthesis, the full-length coding sequence was transformed into Arabidopsis (FIG. 2A) and positive transformants were confirmed by PCR. Sterols were analyzed by GC-MS from rosette leaves of transgenic Arabidopsis overexpressing ZmCYP710A8 with single insertion in the T3 generation. Wild-type (Col-0) produced trace amounts of stigmasterol, while its concentration in transgenic lines ranged from 166.3 to 1415.2 idg g⁻¹ DW (FIG. 2B). The stigmasterol increase in transgenic plants was concomitant with a reduction in sitosterol, suggesting a direct substrate-product relationship. Further, stigmasterol content strongly correlated with ZmCYP710A8 mRNA levels (FIG. 2B), suggesting transcriptional regulation of stigmasterol biosynthesis. The concentration of campesterol, however, remained relatively constant in transgenic lines. The apparent morphology of ZmCYP710A8 overexpressing Arabidopsis plants did not differ from that of wild type plants (FIG. 2C). Taken together, ZmCYP710A8 encodes a sterol C-22 desaturase in maize.

### Example 2: Expression Pattern of ZmCYP710A8 During Development and Hormone Treatments

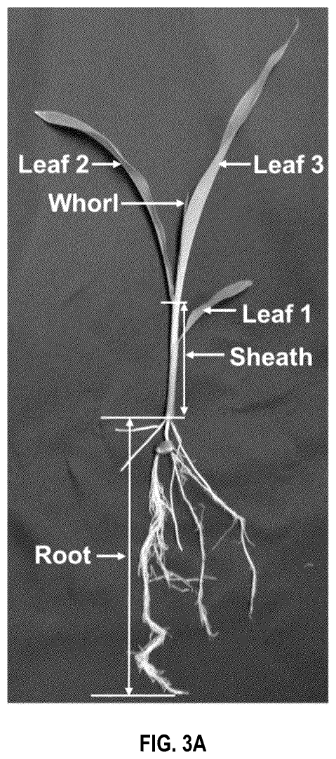

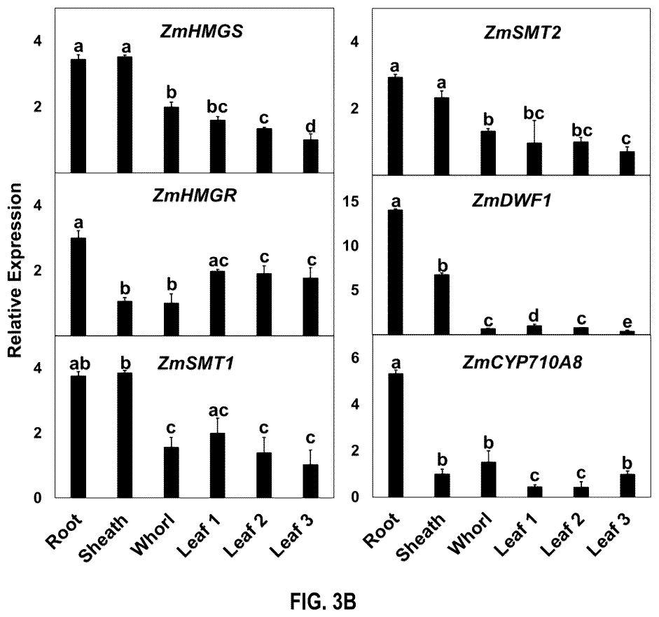

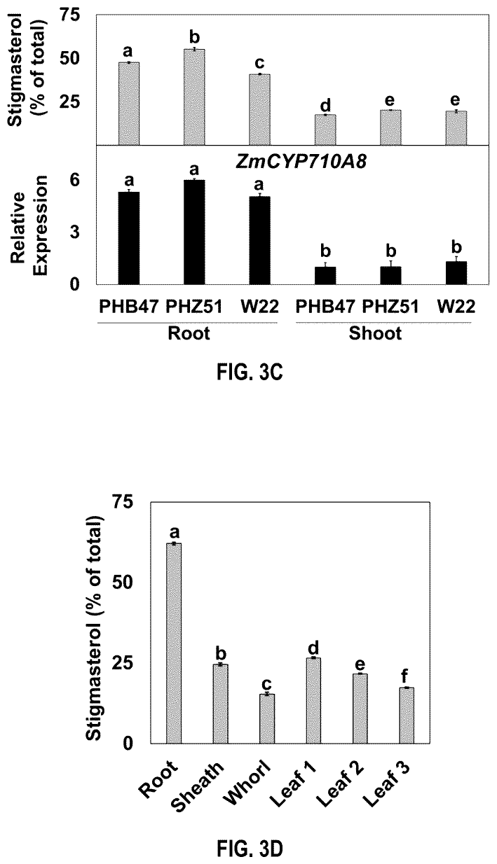

To investigate the developmental expression of ZmCYP710A8, its mRNA was measured in various tissues of maize seedlings (FIG. 3A), along with mRNAs for several key plant sterol biosynthesis genes including HMGS, HMGR, SQS, SMT1, SMT2. Except for DWF1 and ZmCYP710A8, maize encodes multiple copies for HMGS, HMGR, SQS, SMT1, and SMT2. Therefore, PCR primers were designed to capture all molecular transcripts from a given gene family as previously described in N. benthamiana.

Sterol profile of maize seedlings was also measured in addition to mRNA expression (FIG. 3, Table 1). Stigmasterol content was highest in roots compared to aerial tissues in B73 (FIG. 3D) and a similar trend was observed in roots and shoots of additional maize genotypes (FIG. 3C). In contrast, sitosterol concentration displayed an inverse pattern than that of stigmasterol Like stigmasterol, total sterol content was highest in roots followed by aerial tissues (Table 1). The mRNA levels for ZmCYP710A8 and other sterol biosynthesis genes were highest in roots compared to aerial tissues (FIG. 3B), correlating with stigmasterol content (FIG. 3D). ZmCYP710A8 mRNA levels strongly correlated (R²=0.94) with stigmasterol but showed an inverse correlation (R 2=−0.92) with sitosterol in B73 and other maize genotypes (FIG. 3C). The results suggest that the control of stigmasterol biosynthesis is at the transcriptional level, and as suggested (FIG. 2B), a single sterol C-22 desaturase may exist in the maize genome.

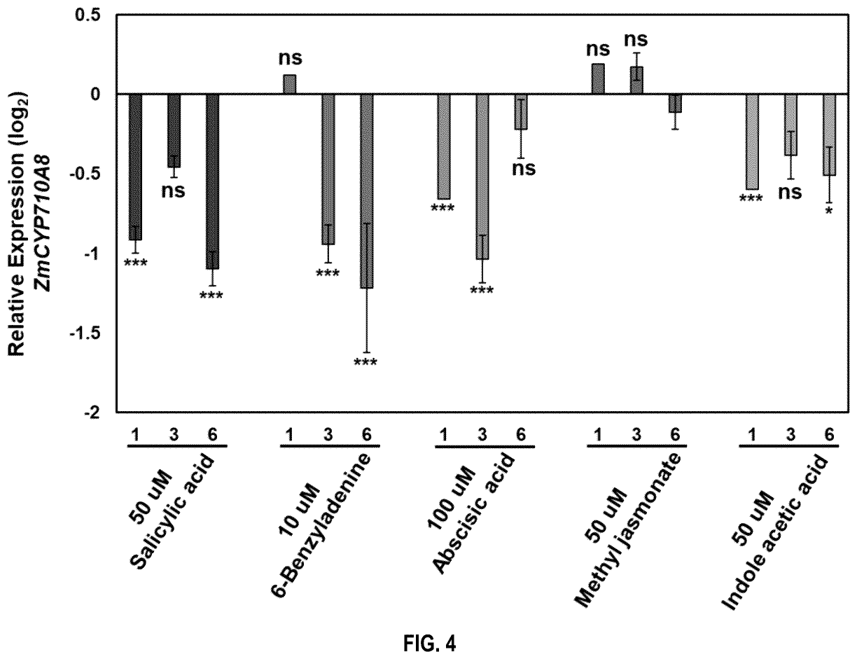

The expression pattern of ZmCYP710A8 in response to phytohormone treatments was examined to study stigmasterol regulation by hormone treatment. All hormone treatments used except methyl jasmonate suppressed the expression of ZmCYP710A8 at indicated time points and concentrations (FIG. 4).

TABLE 1
Free sterols content (μg g⁻¹ DW) in the various maize (B73) organs at V1 stage.

| Tissue | Cholesterol | Campesterol | Sitosterol | Stigmasterol | Total |
|---|---|---|---|---|---|
| Root | Trace | 584.02 ± 19.44 | 492.38 ± 32.97 | 1759.72 ± 58.47 | 2837.88 ± 109.92 |
| Sheath | Trace | 452.29 ± 13.12 | 1215.47 ± 42.99 | 544.26 ± 12.59 | 2213.28 ± 65.02 |
| Whorl | Trace | 242.55 ± 8.84 | 1430.92 ± 98.71 | 304.83 ± 10.00 | 1981.18 ± 115.29 |
| Leaf 1 | Trace | 299.50 ± 7.61 | 720.59 ± 29.16 | 345.48 ± 11.03 | 1296.13 ± 47.37 |
| Leaf 2 | Trace | 222.56 ± 8.68 | 782.48 ± 32.96 | 278.48 ± 9.95 | 1285.09 ± 51.70 |
| Leaf 3 | Trace | 240.16 ± 7.09 | 1081.26 ± 40.1 | 278.72 ± 7.06 | 1601.29 ± 54.23 |

Tissue samples were pooled from 3 individual seedlings in one experiment. Sterols were measured in 4 technical replicates for each experiment. Data are means ± SE from two independent experiments.

### Example 3: ZmCYP710A8 is the Sole Maize Sterol C-22 Desaturase Involved in Stigmasterol Biosynthesis

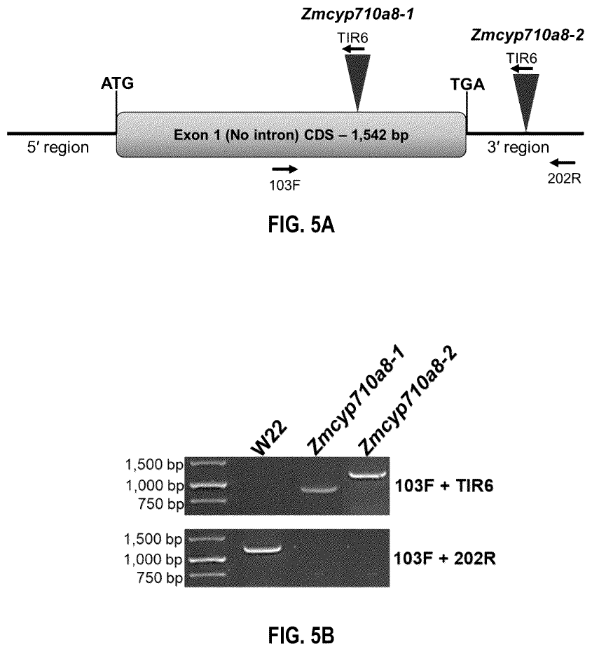

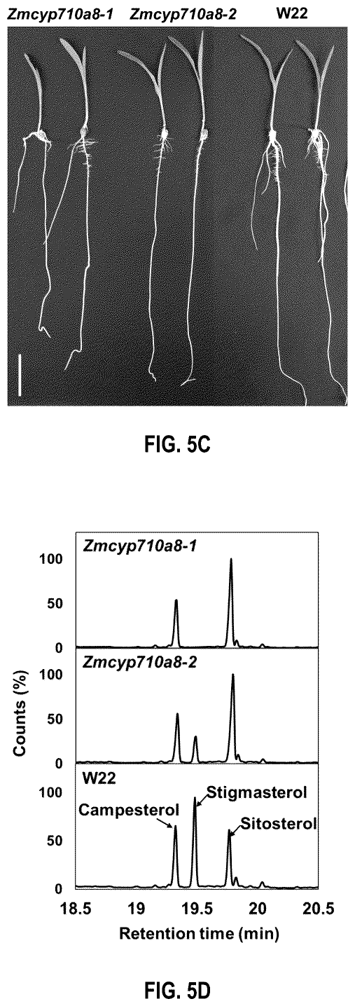

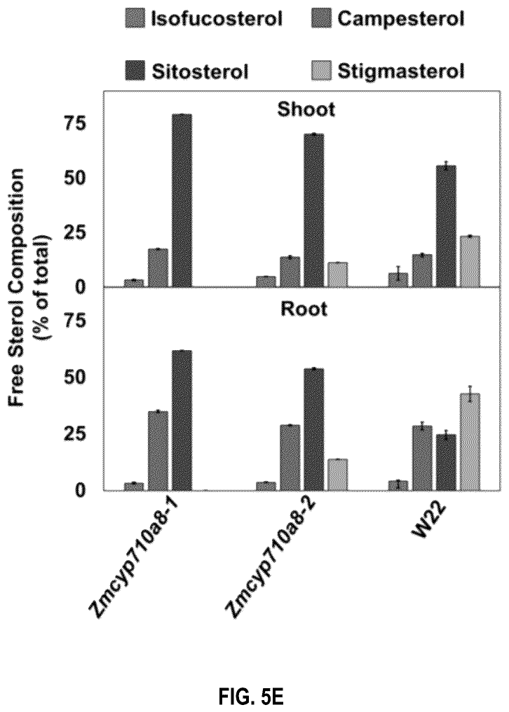

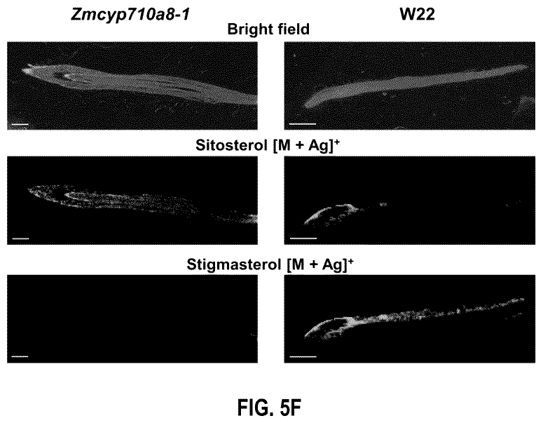

To generate a stigmasterol free system for studying its biological function in maize, plants compromised in stigmasterol production were identified from the maize UniformMu population. PCR analysis led to the identification of transposon insertions in two independent alleles, Zmcyp710a8-1 (mu1034077, UFMu-03461) and Zmcyp710a8-2 (mu1019883, UFMu-00809), within and near the ZmCYP710A8 open-reading frame (FIG. 5A, FIG. 5B). Zmcyp710a8 plants did not show any observable phenotypic defects compared to wild type and they were able to grow and produce seeds under controlled conditions and in the field (FIG. 5C).

Sterol analysis by GC-MS showed the Zmcyp710a8-1 mutant did not produce stigmasterol, but Zmcyp710a8-2 produced 0.3- to 0.5-fold stigmasterol as the W22 wild type (FIG. 5D). Compared to W22, the F₁ progeny from a cross between W22 and Zmcyp710a8-1 (W22/Zmcyp710a8-1) produced 0.8- and 0.6-fold less stigmasterol in roots and shoots, respectively. These results support the phylogenetic analysis and heterologous expression data (FIG. 1, FIG. 2) allowing us to conclude that ZmCYP710A8 is the sole sterol C-22 desaturase in maize.

The composition of free sterols and their intermediates were altered in the Zmcyp710a8-1 and Zmcyp710a8-2 mutants. For instance, intermediate sterols including cycloartenol and isofucosterol were reduced in roots and shoots of Zmcyp710a8-2 relative to W22. In roots, sitosterol was 25% of the total sterols in W22, 54% in Zmcyp710a8-2, and 62% in Zmcyp710a8-1, while in shoots, it was 56% in W22, 70% in Zmcyp710a8-2 and 80% in Zmcyp710a8-1 (FIG. 3C).

Further, campesterol composition was significantly different between W22 and Zmcyp710a8-1; the difference in roots being 28% of total sterols in W22 and 35% in shoots, while in shoots W22 had 14% and Zmcyp710a8-1 had 18%. There was no appreciable difference, however, in campesterol proportions between W22 and Zmcyp710a8-2 (FIG. 5E). Nonetheless, it is noteworthy that the composition of steryl glucosides was proportional to those of free sterols in roots and shoots. Mass spectrometry imaging of wild type and Zmcyp710a8-1 roots indicate that sitosterol is localized to the root tip region while stigmasterol is more broadly distributed (FIG. 5F). Taken together, the disruption of ZmCYP710A8 eliminates stigmasterol production and affects the composition of free sterols and steryl glucosides, and the distribution of sitosterol in roots.

### Example 4: Exogenous Stigmasterol Affects Sterol Gene Transcripts

The positive correlation between stigmasterol content and mRNA for key sterol biosynthesis genes (FIG. 3) suggests stigmasterol may have a stimulatory effect on gene expression. Identification of a stigmasterol-free background in Zmcyp710a8-1 (FIG. 5) provided a system to test whether genes such as HMGR and SMT whose expression in various tissues correlates with stigmasterol content (FIG. 3) might be responsive to exogenously supplied stigmasterol.

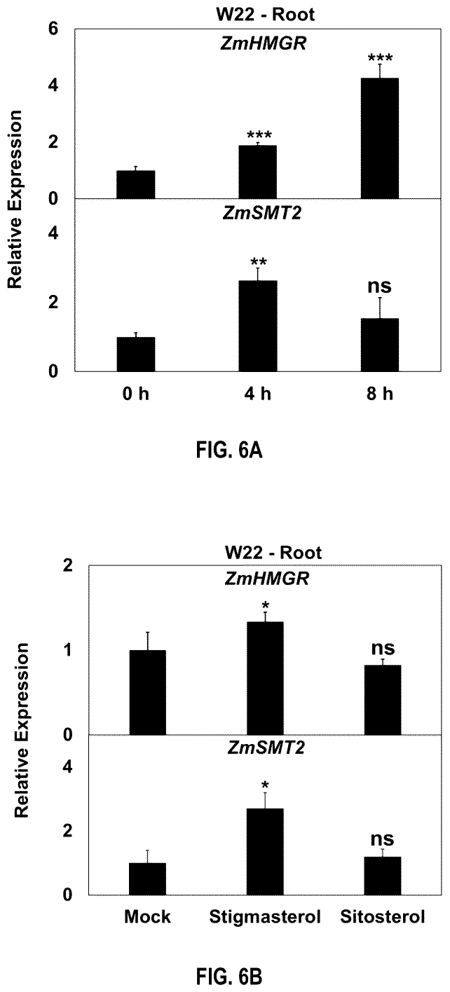

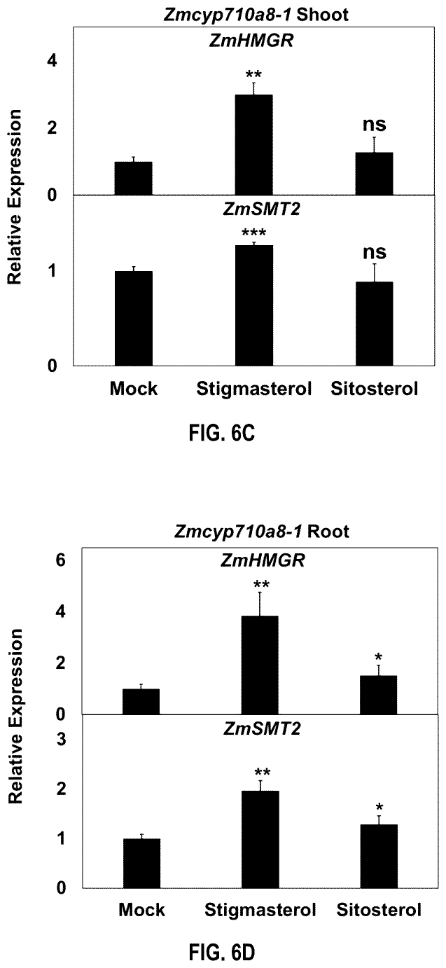

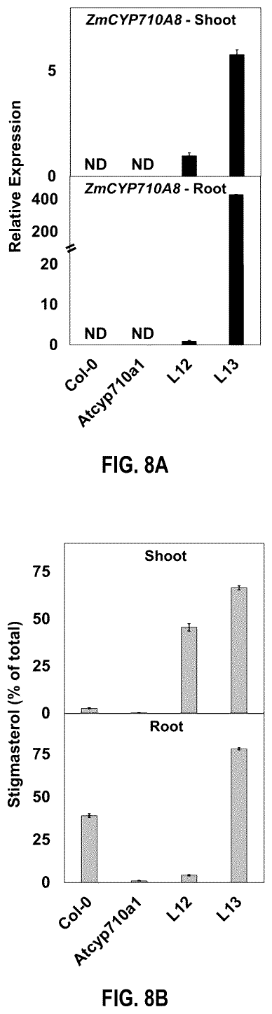

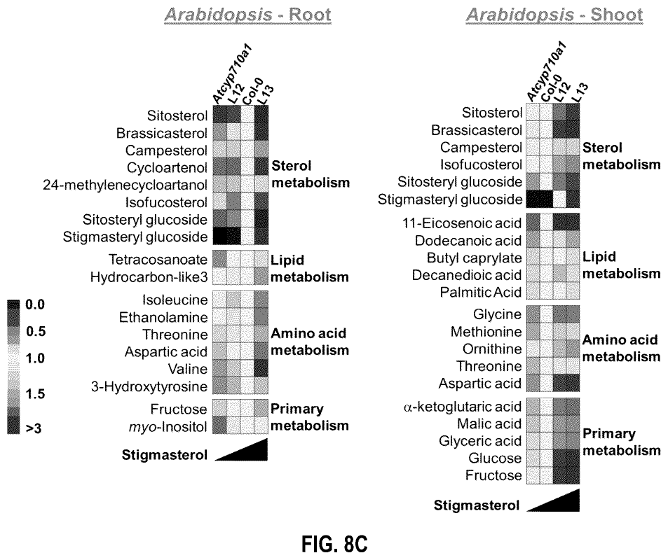

Treatment of W22 roots with exogenous stigmasterol resulted in the induction of ZmHMGR and ZmSMT2 mRNA after 4 and 8 h (FIG. 6A). Although ZmHMGR mRNA remained elevated, ZmSMT2 mRNA slightly decreased after 8 h (FIG. 6A). Exogenous sitosterol, in contrast, did not have a significant impact on ZmHMGR and ZmSMT2 mRNA after 4 h (FIG. 6B) and 8 h.

Similar to the observation in W22, ZmHMGR and ZmSMT2 mRNA levels were higher in roots and shoots of Zmcyp710a8-1 treated with stigmasterol (FIG. 6C and FIG. 6D). Sitosterol, however, had a modest effect on ZmHMGR and ZmSMT2 mRNA in roots of Zmcyp710a8-1 (FIG. 6C and FIG. 6D). The impact of stigmasterol on mRNA for ZmHMGR and ZmSMT2 suggests that stigmasterol might modulate transcripts for plant sterol biosynthesis genes.

### Example 5: Stigmasterol Impacts Global Cellular Metabolites in Maize

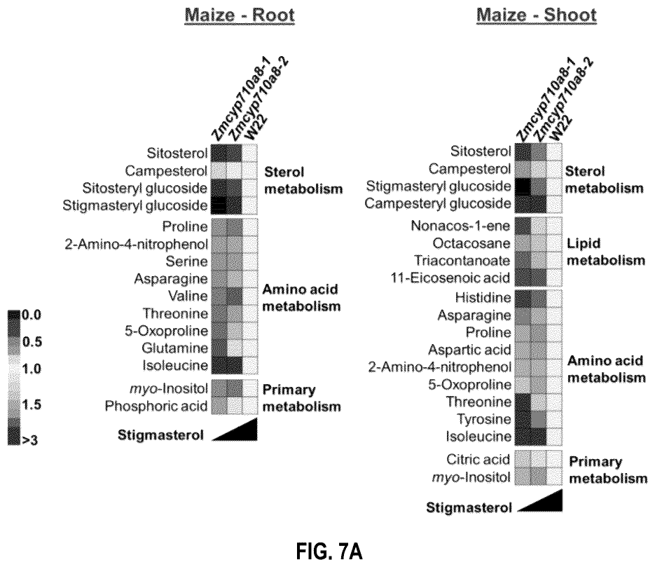

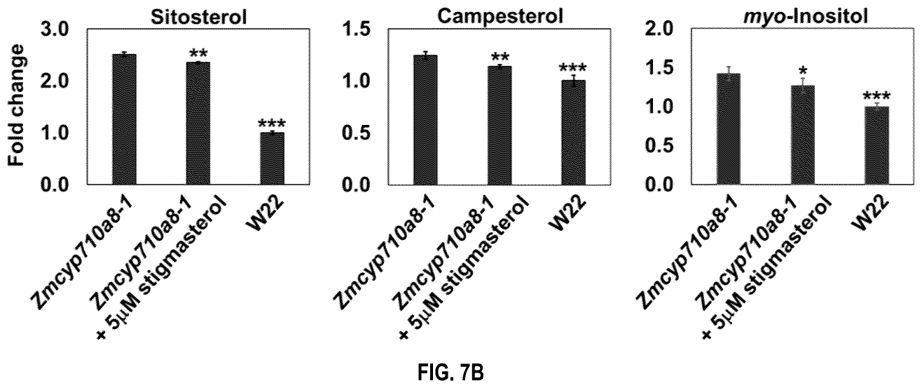

To identify cellular metabolites influenced by stigmasterol, non-targeted GC/MS metabolite analysis of roots and shoots of maize Zmcyp710a8 and wild type was used. A total of 29 molecular features (13 known and 16 unknown molecular features) were identified in roots and 31 features (17 known and 14 unknown molecular features) in shoots (FIG. 7). The concentration of molecular features correlated with stigmasterol content among genotypes in maize and the features belonged to similar classes including sterol, amino acid, primary metabolism, and unknown features in roots and shoots (FIG. 7).

Metabolites, such as glutamine and an unknown feature with retention index (RI) 1796.8 in Zmcyp710a8-1 roots were 1.6- and 1.9-fold higher, respectively, compared to W22 (FIG. 7A). Threonine and tyrosine in shoots were 1.6- and 1.7-fold higher in Zmcyp710a8-1 compared to W22 (FIG. 7A), respectively. Among genotypes, changes in metabolites correlated with stigmasterol content (FIG. 7A). For instance, threonine and myo-inositol showed a negative correlation with stigmasterol content in roots and shoots. Other metabolites that negatively correlated with stigmasterol were asparagine, 5-oxoproline and glutamine in roots, and tyrosine and triacontanoate in shoots (FIG. 7A). Furthermore, unknown features with RI 1286.4 in roots and shoots; 1475.7, 1796.8, 1178.2 and 1600.6 in roots, and 2097.6 and 3074.5 in shoots were among the metabolites inversely correlated with stigmasterol. Similarly, sitosteryl and campesteryl glucosides in roots and shoots, respectively, negatively correlated with stigmasterol (FIG. 7A). Compounds that positively correlated with stigmasterol were stigmasteryl glucoside in roots and shoots and histidine, asparagine and nonacos-1-ene in shoots (FIG. 7A). Unknown features with RIs of 1969.8 in roots and shoots; 2062.7, 1621.8 and 1720.0 in roots; 1545.3 and 1483.0 in shoots were also directly correlated with stigmasterol.

Since changes in metabolites among the genotypes correlated with stigmasterol content, it was reasoned that exogenous stigmasterol may reverse the metabolite levels in Zmcyp710a8-1 mutant to wild type levels. Treatment of the Zmcyp710a8-1 null mutant with exogenous stigmasterol shifted the levels of sitosterol, campesterol, and myo-inositol toward W22 levels (FIG. 7B), suggesting a complementation effect.

### Example 6: Stigmasterol Modification Impacts Global Cellular Metabolites in Arabidopsis

To test whether stigmasterol impacts cellular metabolism in a dicot species, the study was extended to Arabidopsis. The Atcyp710a1 mutant produces trace amounts of stigmasterol in roots (44.74 μg g⁻¹ DW) and shoots (12.29 μg g⁻¹ DW) providing a good system to test the impact of restoring or increasing stigmasterol production on cellular metabolism (Table 2). The full-length ZmCYP710A8 ORF with the 35S viral promoter (FIG. 2A) was transformed into Atcyp710a1 to obtain several lines expressing ZmCYP710A8. Two lines (L12 and L13) with single insertion of the transgene in T4 generation were selected for further analysis. There was a direct correlation between stigmasterol content and ZmCYP710A8 mRNA in each of the transgenic lines (FIG. 8A, FIG. 8B). In line L13, ZmCYP710A8 mRNA and stigmasterol were high in roots (3417.28 μg g⁻¹ DW) and shoots (2766.02 μg g⁻¹ DW), while their sitosterol levels were reduced consistent with the presence of CYP710A1 in Col-0 and CYP710A8 in L13. In contrast, ZmCYP710A8 mRNA and stigmasterol concentration of line L12 in roots (182.76 μg g⁻¹ DW) and shoots (1824.12 μg g⁻¹ DW) was reciprocal to that of the wild type (FIG. 8A, FIG. 8B; Table 2). Line L12 had a higher sitosterol content in roots as in Atcyp710a1 consistent with low CYP710A8 mRNA in L12 and reduced CYP710A1 activity in Atcyp710a1. In roots and shoots, sitosterol and campesterol followed an inverse trend with stigmasterol while brassicasterol positively correlated with stigmasterol (Table 2). The campesterol content in shoots was also similar between Atcyp710a1 and Col-0 but lower in L12 and L13. Brassicasterol levels were comparable among Atcyp710a1, Col-0 and L12 (69.44 to 95.96 μg g⁻¹ DW) but were considerably higher in L13 (136.07 μg g⁻¹ DW) (Table 2).

Non-targeted GC/MS metabolite analysis was also used to study cellular metabolites in the Atcyp710a1 mutant, Col-0, L12 and L13. Molecular features with significant changes among Arabidopsis genotypes were identified as in maize. A correlation between Arabidopsis genotypes and the concentration of molecular features was observed. Metabolite concentrations among the Arabidopsis genotypes correlated with their stigmasterol content like maize (FIG. 8C). A total of 39 molecular features (15 known and 24 unknown molecular features) were identified in roots and 28 features (17 known and 11 unknown molecular features) in shoots (FIG. 8C). Sitosteryl glucoside levels in roots and shoots were negatively correlated with stigmasterol, while stigmasteryl glucoside was positively correlated (FIG. 8C). Methionine was 0.7-fold and RI 2288.9 was 2.4-fold in shoots of Atcyp710a1 as Col-0 (FIG. 8C), and myo-inositol in roots and threonine were affected in both Arabidopsis and maize shoots. It is noteworthy that, Zmcyp710a8-1 and Zmcyp7108-2 have higher level of several amino acids in roots and shoots. Conversely, the Atcyp710a1 mutant produces comparable levels of several amino acids in roots and shoots, but there is an upward shift in amino acid levels when stigmasterol is increased as in L13 (FIG. 8C).

Metabolites such as tetracosanoate and molecular feature RI 2996.5 were 1.4- and 1.6-fold higher, respectively, in Atcyp710a1 roots compared to Col-0 and L13 (FIG. 8C). Several other metabolites were also affected in Arabidopsis correlating with stigmasterol, fructose and aspartic acid contents having negative correlations with stigmasterol in roots and shoots (FIG. 8C). Other compounds that negatively correlated with stigmasterol in roots were 24-methylenecycloartanol, tetracosanoate, and several unknown features in root with retention indices (RI) 1743.4, 1853.8, 2430.4, 2686.9, 2815.5, 2996.5, 3313.5, 3436.2, 3574.1, and 3616.1 (FIG. 8C). The metabolites positively correlated with stigmasterol content in roots were cycloartenol, hydrocarbon-like3 (an unknown molecular feature with spectral similarity to known normal-chain hydrocarbons), isoleucine and 3-hydroxytyrosine, and unknown features with RIs 1496.8, 1578.5, and 2382.9 (FIG. 8C).

In shoots, isofucosterol, ornithine, α-ketoglutaric acid, malic acid, glyceric acid and a few unknown features with RIs 1965.6, 2288.9, and 2430.4 were inversely correlated with stigmasterol (FIG. 8C). On the other hand, there was a direct correlation between stigmasterol and several metabolites such as 11-eicosenoic acid, methionine, glucose, and unknown features with RI 1525.6, and 1945.7 in shoots (FIG. 8C).

TABLE 2
Free sterols content (μg g⁻¹ DW) in Arabidopsis seedlings.

| Tissue | Free Sterols | Atcyp710a1 | L12 | WT (Col-0) | L13 |
|---|---|---|---|---|---|
| Root | Campesterol | 502.81 ± 55.67 | 498.35 ± 44.94 | 592.99 ± 62.90 | 383.37 ± 40.46 |
| | Brassicasterol | 44.13 ± 11.25 | 67.33 ± 19.70 | 72.28 ± 8.62 | 230.06 ± 28.19 |
| | Sitosterol | 3612.07 ± 123.24 | 3493.94 ± 97.72 | 2165.25 ± 55.60 | 339.73 ± 18.37 |
| | Stigmasterol* | 44.74 ± 5.61 | 182.76 ± 11.78 | 1826.08 ± 129.59 | 3417.28 ± 128.40 |
| | Total | 4203.76 ± 191.29 | 4242.38 ± 136.35 | 4656.6 ± 201.53 | 4370.44 ± 165.5 |
| | | Atcyp710a1 | WT (Col-0) | L12 | L13 |
| Shoot | Campesterol | 585.30 ± 19.09 | 603.50 ± 5.78 | 529.78 ± 41.48 | 518.37 ± 12.78 |
| | Brassicasterol | 69.44 ± 17.08 | 72.76 ± 5.90 | 95.96 ± 15.47 | 136.07 ± 21.93 |
| | Sitosterol | 3301.29 ± 107.65 | 3371.78 ± 172.62 | 1553.63 ± 79.86 | 736.19 ± 58.47 |
| | Stigmasterol* | 12.29 ± 5.96 | 109.73 ± 13.44 | 1824.12 ± 156.02 | 2766.02 ± 171.34 |
| | Total | 3968.31 ± 146.3 | 4157.78 ±154.21 | 4003.49 ± 232.39 | 4156.65 ± 217.06 |

### Example 7: Zmcyp710a8 Mutation Increases Healthy Phytosterol Content in Maize Seeds

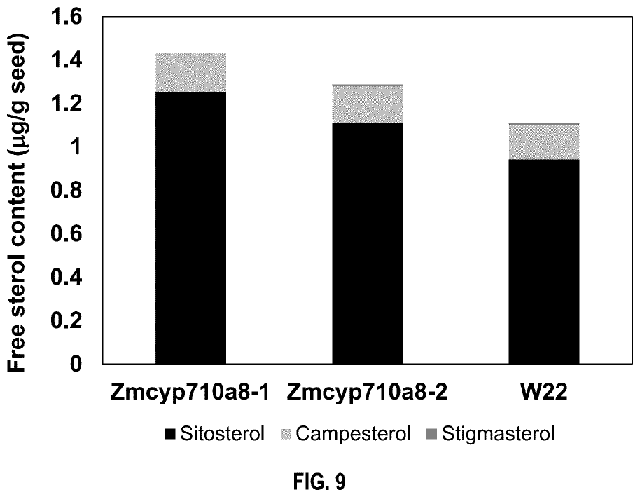

Healthy phytosterol (sitosterol) is increased by Zmcyp710a8 mutation in maize seeds (FIG. 9). Free sterol content in seeds were measured using Gas Chromatography-Mass Spectrometry method. Total sterol content is increased in Zmcyp710a8 mutants compared to wild type (W22). Specifically, sitosterol is increased to 1.5 and 1.2 folds in Zmcyp710a8-1 and Zmcyp710a8-2, respectively, compared to wildtype. Stigmasterol is absent in Zmcyp710a8-1 and is present only in trace amounts in Zmcyp710a8-2. Campesterol levels remain the same in all three genotypes.

### Example 8: Zmcyp710a8 Mutation Increases Hard Endosperm (Food Grade) in Maize Seeds

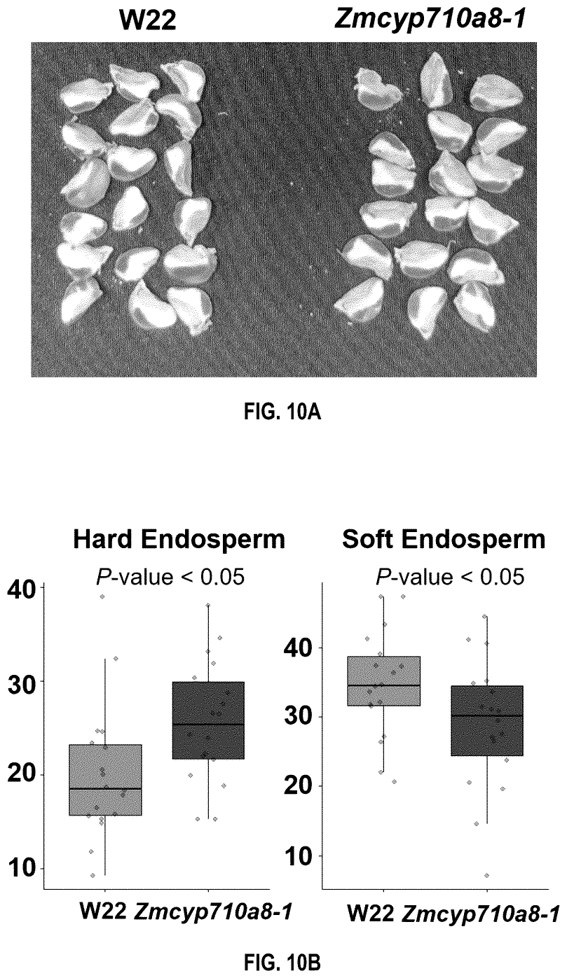

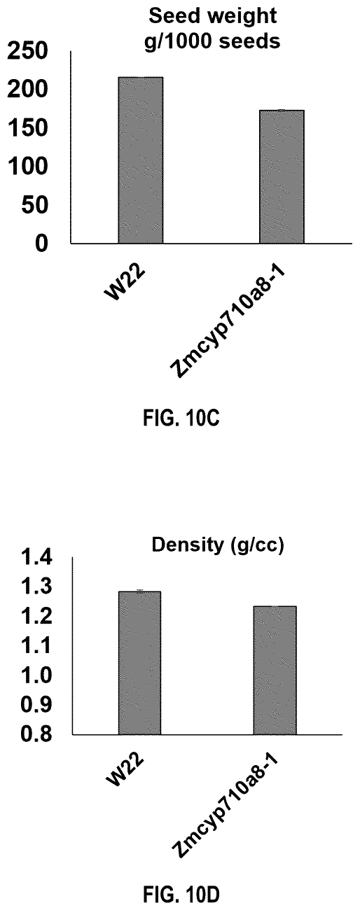

Hard endosperm (improved food grade) of maize seeds is increased by Zmcyp710a8-1 mutation (FIG. 10). Zmcyp710a8-1 kernels display more hard endosperm than soft endosperm compared to W22 (wild type) kernels. Seed weight and density are reduced in Zmcyp710a8-1 kernels compared to wild type indicative of increased hard endosperm.

### REFERENCES

Aboobucker, S. I., and Suza, W. P. (2019). Why do plants convert sitosterol to stigmasterol? Frontiers in Plant Science 10, 354. doi: 10.3389/fpls.2019.00354.
Aboobucker, S. I., Suza, W. P., and Lorence, A. (2017). Characterization of two Arabidopsis L-gulono-1,4-lactone oxidases, AtGulLO3 and AtGulLO5, involved in ascorbate biosynthesis. Reactive Oxygen Species 4(12), 389-417. doi: 10.20455/ros.2017.861.
Arnqvist, L., Persson, M., Jonsson, L., Dutta, P. C., and Sitbon, F. (2008). Overexpression of CYP710A1 and CYP710A4 in transgenic Arabidopsis plants increases the level of stigmasterol at the expense of sitosterol. Planta 227(2), 309-317. doi: 10.1007/s00425-007-0618-8.
Atsumi, G., Kagaya, U., Tabayashi, N., and Matsumura, T. (2018). Analysis of the mechanisms regulating the expression of isoprenoid biosynthesis genes in hydroponically-grown Nicotiana benthamiana plants using virus-induced gene silencing. Scientific Reports 8(1), 14804-14811. doi: 10.1038/s41598-018-32901-5.
Bach, T. J. (2016). Secondary metabolism: High cholesterol in tomato. Nature Plants 3(1), 16213. doi: 10.1038/nplants.2016.213.
Benveniste, P. (2002). Sterol Metabolism. The Arabidopsis Book 1, e0004. doi: 10.1199/tab.0004.
Best, N. B., Hartwig, T., Budka, J., Fujioka, S., Johal, G., Schulz, B., et al. (2016). nana plant2 encodes a maize ortholog of the Arabidopsis brassinosteroid biosynthesis gene DWARF1, identifying developmental interactions between brassinosteroids and gibberellins. Plant Physiology 171(4), 2633-2647. doi: 10.1104/pp. 16.00399.
Betts, H., and Moore, I. (2003). Plant cell polarity: The ins-and-outs of sterol transport. Current Biology 13(19), R781-R783. doi: 10.1016/j.cub.2003.09.023.
Bhatt, P. N., and Bhatt, D. P. (1984). Regulation of sterol biosynthesis in Solanum species. Journal of Experimental Botany 35(155), 890-896.
Bishop, G. J., and Koncz, C. (2002). Brassinosteroids and plant steroid hormone signaling. The Plant Cell 14 (Suppl), S97-S110. doi: 10.1105/tpc.001461.
Boyes, D. C., Zayed, A. M., Ascenzi, R., McCaskill, A. J., Hoffman, N. E., Davis, K. R., et al. (2001). Growth stage-based phenotypic analysis of Arabidopsis: a model for high throughput functional genomics in plants. The Plant Cell 13(7), 1499-1510. doi: 10.1105/TPC.010011.
Cabianca, A., Müller, L., Pawlowski, K., and Dahlin, P. (2021). Changes in the plant β-sitosterol/stigmasterol ratio caused by the plant parasitic nematode Meloidogyne incognita. Plants (Basel) 10(2), 292. doi: 10.3390/plants10020292.
Carland, F., Fujioka, S., and Nelson, T. (2010). The sterol methyltransferases SMT1, SMT2, and SMT3 influence Arabidopsis development through nonbrassinosteroid products. Plant Physiology 153(2), 741-756. doi: 10.1104/pp. 109.152587.
Carland, F. M., Fujioka, S., Takatsuto, S., Yoshida, S., and Nelson, T. (2002). The identification of CVP1 reveals a role for sterols in vascular patterning. The Plant Cell 14(9), 2045-2058.
Chappell, J. (1995). Biochemistry and Molecular Biology of the Isoprenoid Biosynthetic Pathway in Plants. Annual Review of Plant Physiology and Plant Molecular Biology 46(1), 521-547. doi: 10.1146/annurev.pp. 46.060195.002513.
Chappell, J. (2002). The genetics and molecular genetics of terpene and sterol origami. Current Opinion Plant Biology 5(2), 151-157.
Choe, S., Noguchi, T., Fujioka, S., Takatsuto, S., Tissier, C. P., Gregory, B. D., et al. (1999). The Arabidopsis dwf7/stel mutant is defective in the delta? sterol C-5 desaturation step leading to brassinosteroid biosynthesis. The Plant Cell 11(2), 207-221.
Clough, S. J., and Bent, A. F. (1998). Floral dip: a simplified method for Agrobacterium-mediated transformation of Arabidopsis thaliana. The Plant Journal 16(6), 735-743. doi: 10.1046/j.1365-313x.1998.00343.x.
Clouse, S. D. (2002). Arabidopsis mutants reveal multiple roles for sterols in plant development. The Plant Cell 14(9), 1995-2000. doi: 10.1105/tpc.140930.
Cooper, M. K., Wassif, C. A., Krakowiak, P. A., Taipale, J., Gong, R., Kelley, R. I., et al. (2003). A defective response to Hedgehog signaling in disorders of cholesterol biosynthesis. Nature Genetics 33(4), 508-513. doi: 10.1038/ng1134.
Dalal, J., Lewis, D. R., Tietz, O., Brown, E. M., Brown, C. S., Palme, K., et al. (2016). ROSY1, a novel regulator of gravitropic response is a stigmasterol binding protein. Journal of Plant Physiology 196-197, 28-40. doi: 10.1016/j.jplph.2016.03.011.
Devarenne, T. P., Ghosh, A., and Chappell, J. (2002). Regulation of squalene synthase, a key enzyme of sterol biosynthesis, in tobacco. Plant Physiology 129(3), 1095-1106. doi: 10.1104/pp. 001438.
Diener, A. C., Li, H., Zhou, W., Whoriskey, W. J., Nes, W. D., and Fink, G. R. (2000). Sterol methyltransferase 1 controls the level of cholesterol in plants. The Plant Cell 12(6), 853-870.
Dufresne, M., Thomas, A. I., Breault-Turcot, J., Masson, J.-F.o., and Chaurand, P. (2013). Silver-assisted laser desorption ionization for high spatial resolution imaging mass spectrometry of olefins from thin tissue sections. Analytical Chemistry 85(6), 3318-3324. doi: 10.1021/ac3037415.
Edgar, R. C. (2004). MUSCLE: multiple sequence alignment with high accuracy and high throughput. Nucleic Acids Research 32(5), 1792-1797. doi: 10.1093/nar/gkh340.
Edwards, P. A., and Ericsson, J. (1999). Sterols and isoprenoids: signaling molecules derived from the cholesterol biosynthetic pathway. Annual Review of Biochemistry 68, 157.
Fiehn, O., Kopka, J., Trethewey, R. N., and Willmitzer, L. (2000). Identification of uncommon plant metabolites based on calculation of elemental compositions using gas chromatography and quadrupole mass spectrometry. Analytical Chemistry 72(15), 3573-3580. doi: 10.1021/ac991142i.
Friedrichsen, D., and Chory, J. (2001). Steroid signaling in plants: from the cell surface to the nucleus. Bioessays 23(11), 1028-1036. doi: 10.1002/bies.1148.
Fujioka, S., Li, J., Choi, Y. H., Seto, H., Takatsuto, S., Noguchi, T., et al. (1997). The Arabidopsis deetiolated2 mutant is blocked early in brassinosteroid biosynthesis. The Plant Cell 9(11), 1951-1962. doi: 10.1105/tpc.9.11.1951.
Fukuda, H. (2004). Signals that control plant vascular cell differentiation. Nature Reviews Molecular Cell Biology 5(5), 379-391. doi: 10.1038/nrm1364.
Garcia, L. G. (2019). Unveiling the biological role of stigmasterol biosynthesis in tomato plants. Doctoral PhD thesis, Universitat de Barcelona.
Goldstein, J. L., and Brown, M. S. (1990). Regulation of the mevalonate pathway. Nature 343(6257), 425-430. doi: 10.1038/343425a0.
Grandmougin-Ferjani, A., Schuler-Muller, I., and Hartmann, M. A. (1997). Sterol modulation of the plasma membrane H⁺-ATPase activity from corn roots reconstituted into soybean lipids. Plant Physiology 113(1), 163-174. doi: 10.1104/pp. 113.1.163.
Griebel, T., and Zeier, J. (2010). A role for β-sitosterol to stigmasterol conversion in plant—pathogen interactions. The Plant Journal 63(2), 254-268. doi: 10.1111/j.1365-313X.2010.04235.x.
Grosjean, K., Mongrand, S., Beney, L., Simon-Plas, F., and Gerbeau-Pissot, P. (2015). Differential effect of plant lipids on membrane organization: specificities of phytosphingolipids and phytosterols. The Journal of Biological Chemistry 290(9), 5810-5825. doi: 10.1074/jbc.M114.598805.
Hartmann, M.-A. (1998). Plant sterols and the membrane environment. Trends in Plant Science 3(5), 170-175. doi: 10.1016/S1360-1385(98)01233-3.
Hartmann, M.-A. (2004). “Sterol metabolism and functions in higher plants,” in Lipid metabolism and membrane biogenesis, ed. G. Daum. (Berlin, Heidelberg: Springer Berlin Heidelberg), 183-211.
He, J.-X., Fujioka, S., Li, T.-C., Kang, S. G., Seto, H., Takatsuto, S., et al. (2003). Sterols regulate development and gene expression in Arabidopsis. Plant Physiology 131(3), 1258-1269. doi: 10.1104/pp. 014605.
Heupel, R. C., Sauvaire, Y., Le, P. H., Parish, E. J., and Nes, W. D. (1986). Sterol composition and biosynthesis in sorghum: Importance to developmental regulation. Lipids 21(1), 69-75. doi: 10.1007/BF02534305.
Holsters, M., de Waele, D., Depicker, A., Messens, E., van Montagu, M., and Schell, J. (1978). Transfection and transformation of Agrobacterium tumefaciens. Molecular and General Genetics 163(2), 181-187. doi: 10.1007/bf00267408.
Jang, J. C., Fujioka, S., Tasaka, M., Seto, H., Takatsuto, S., Ishii, A., et al. (2000). A critical role of sterols in embryonic patterning and meristem programming revealed by the fackel mutants of Arabidopsis thaliana. Genes and development 14(12), 1485-1497. doi: 10.1101/gad.14.12.1485.

Jones, D. T., Taylor, W. R., and Thornton, J. M. (1992). The rapid generation of mutation data matrices from protein sequences. Computer Application in the Biosciences 8(3), 275-282. doi: 10.1093/bioinformatics/8.3.275.
Joshi, V., Joung, J.-G., Fei, Z., and Jander, G. (2010). Interdependence of threonine, methionine and isoleucine metabolism in plants: accumulation and transcriptional regulation under abiotic stress. Amino Acids 39(4), 933-947. doi: 10.1007/s00726-010-0505-7.
Jun, J. H., Song, Z., Liu, Z., Nikolau, B., Yeung, E. S., and Lee, Y. J. (2010). High-spatial and high-mass resolution imaging of surface metabolites of Arabidopsis thaliana by laser desorption-ionization mass spectrometry using colloidal silver. Analytical Chemistry 82(8), 3255-3265. doi: 10.1021/ac902990p.
Kemp, R. J., Goad, L. J., and Mercer, E. I. (1967). Changes in the levels and composition of the esterified and unesterified sterols of maize seedlings during germination. Phytochemistry 6(12), 1609-1615. doi: 10.1016/S0031-9422(00)82892-7.
Koek, M. M., Muilwijk, B., van der Werf, M. J., and Hankemeier, T. (2006). Microbial metabolomics with gas chromatography/mass spectrometry. Analytical Chemistry 78(4), 1272-1281. doi: 10.1021/ac051683+.
Kumar, B., Abdel-Ghani, A. H., Reyes-Matamoros, J., Hochholdinger, F., and Lübberstedt, T. (2012). Genotypic variation for root architecture traits in seedlings of maize (Zea mays L.) inbred lines. Plant Breeding 131(4), 465-478. doi: 10.1111/j.1439-0523.2012.01980.x.
Kumar, S., Stecher, G., Li, M., Knyaz, C., and Tamura, K. (2018). MEGA X: Molecular evolutionary genetics analysis across computing platforms. Molecular Biology and Evolution 35(6), 1547-1549. doi: 10.1093/molbev/msy096.
Li, Z., Cheng, B., Yong, B., Liu, T., Peng, Y., Zhang, X., et al. (2019). Metabolomics and physiological analyses reveal β-sitosterol as an important plant growth regulator inducing tolerance to water stress in white clover. Planta 250(6), 2033-2046. doi: 10.1007/s00425-019-03277-1.
Lindsey, K., Pullen, M. L., and Topping, J. F. (2003). Importance of plant sterols in pattern formation and hormone signalling. Trends in Plant Science 8(11), 521-525. doi: 10.1016/j.tplants.2003.09.012.
Liu, P., McCarty, D. R., and Koch, K. E. (2016). Transposon mutagenesis and analysis of mutants in UniformMu maize (Zea mays). Current Protocols in Plant Biology 1(3), 451-465.
Loewus, F. A., and Murthy, P. P. N. (2000). myo-Inositol metabolism in plants. Plant science (Limerick) 150(1), 1-19. doi: 10.1016/s0168-9452(99)00150-8.
Lorence, A., Chevone, B. I., Mendes, P., and Nessler, C. L. (2004). myo-Inositol oxygenase offers a possible entry point into plant ascorbate biosynthesis. Plant Physiology 134(3), 1200-1205. doi: 10.1104/pp. 103.033936.
Louis, J., Basu, S., Varsani, S., Castano-Duque, L., Jiang, V., Williams, W. P., et al. (2015). Ethylene contributes to maize insect resistance1-mediated maize defense against the phloem sap-sucking corn leaf aphid. Plant Physiology 169(1), 313-324. doi: 10.1104/pp. 15.00958.
Marigo, V., and Tabin, C. J. (1996). Regulation of patched by sonic hedgehog in the developing neural tube. Proceedings of the National Academy of Sciences 93(18), 9346-9351. doi: 10.1073/pnas.93.18.9346.
Masucci, J. D., Rerie, W. G., Foreman, D. R., Zhang, M., Galway, M. E., Marks, M. D., et al. (1996). The homeobox gene GLABRA2 is required for position-dependent cell differentiation in the root epidermis of Arabidopsis thaliana. Development 122(4), 1253-1260.
McCarty, D. R., Mark Settles, A., Suzuki, M., Tan, B. C., Latshaw, S., Porch, T., et al. (2005). Steady-state transposon mutagenesis in inbred maize. The Plant Journal 44(1), 52-61. doi: 10.1111/j.1365-313x.2005.02509.x.
McConnell, J. R., Emery, J., Eshed, Y., Bao, N., Bowman, J., and Barton, M. K. (2001). Role of PHABULOSA and PHAVOLUTA in determining radial patterning in shoots. Nature 411(6838), 709. doi: 10.1038/35079635.
Morikawa, T., Mizutani, M., Aoki, N., Watanabe, B., Saga, H., Saito, S., et al. (2006). Cytochrome P450 CYP710A encodes the sterol C-22 desaturase in Arabidopsis and tomato. The Plant Cell 18(4), 1008-1022. doi: 10.1105/tpc.105.037012.
Morikawa, T., Saga, H., Hashizume, H., and Ohta, D. (2009). CYP710A genes encoding sterol C22-desaturase in Physcomitrella patens as molecular evidence for the evolutionary conservation of a sterol biosynthetic pathway in plants. Planta 229(6), 1311-1322. doi: 10.1007/s00425-009-0916-4.
Murashige, T., and Skoog, F. (1962). A revised medium for rapid growth and bio assays with tobacco tissue cultures. Physiologia Plantarum 15(3), 473-497. doi: 10.1111/j.1399-3054.1962.tb08052.x.
Nakamoto, M., Schmit, A. C., Heintz, D., Schaller, H., and Ohta, D. (2015). Diversification of sterol methyltransferase enzymes in plants and a role for β-sitosterol in oriented cell plate formation and polarized growth. The Plant Journal 84(5), 860-874. doi: 10.1111/tpj.13043.
Nelson, D. R. (2009). The cytochrome P450 homepage. Human Genomics 4(1), 59-65. doi: 10.1186/1479-7364-4-1-59.
Noguchi, T., Fujioka, S., Takatsuto, S., Sakurai, A., Yoshida, S., Li, J., et al. (1999). Arabidopsis det2 is defective in the conversion of (24R)-24-methylcholest-4-En-3-one to (24R)-24-Methyl-5α-cholestan-3-one in brassinosteroid biosynthesis. Plant Physiology 120(3), 833-840. doi: 10.1104/pp. 120.3.833.
Nomura, T., Takatsuto, S., Reid, J. B., and Yokota, T. (1999). Brassinosteroid/sterol synthesis and plant growth as affected by Ika and Ikb mutations of pea. Plant Physiology 119(4), 1517-1526. doi: 10.1104/pp. 119.4.1517.
Ortiz-Masia, D., Perez-Amador, M. A., Carbonell, J., and Marcote, M. J. (2007). Diverse stress signals activate the C1 subgroup MAP kinases of Arabidopsis. FEBS Letters 581(9), 1834-1840. doi: 10.1016/j.febslet.2007.03.075.
Ostlund, R. E. (2002). Phytosterols in human nutrition. Annual Review of Nutrition 22(1), 533-549. doi: 10.1146/annurev.nutr.22.020702.075220.
Otsuga, D., Deguzman, B., Prigge, M. J., Drews, G. N., and Clark, S. E. (2001). REVOLUTA regulates meristem initiation at lateral positions. The Plant Journal 25(2), 223-236. doi: 10.1111/j.1365-313X.2001.00959.x.
Pfaffl, M. W. (2001). A new mathematical model for relative quantification in real-time RT-PCR. Nucleic Acids Research 29(9), e45. doi: 10.1093/nar/29.9.e45.
Phillips, K. M., Ruggio, D. M., and Ashraf-Khorassani, M. (2005). Analysis of steryl glucosides in foods and dietary supplements by solid-phase extraction and GC. Journal of Food Lipids 12(2), 124-140. doi: 10.1111/j.1745-4522.2005.00011.x.
Porter, F. D. (2002). Malformation syndromes due to inborn errors of cholesterol synthesis. The Journal of Clinical Investigation 110(6), 715-724. doi: 10.1172/JCI200216386.
Pratelli, R., and Pilot, G. (2014). Regulation of amino acid metabolic enzymes and transporters in plants. Journal of Experimental Botany 65(19), 5535-5556. doi: 10.1093/jxb/eru320.
Ritchie, S. W., Hanway, J. J., and Benson, G. O. (2005). How a corn plant develops. Ames, Iowa: Iowa State University of Science and Technology, Cooperative Extension Service.
Sawai, S., Ohyama, K., Yasumoto, S., Seki, H., Sakuma, T., Yamamoto, T., et al. (2014). Sterol Side Chain Reductase 2 Is a Key Enzyme in the Biosynthesis of Cholesterol, the Common Precursor of Toxic Steroidal Glycoalkaloids in Potato. The Plant Cell 26(9), 3763-3774. doi: 10.1105/tpc.114.130096.
Schaeffer, A., Bronner, R., Benveniste, P., and Schaller, H. (2001). The ratio of campesterol to sitosterol that modulates growth in Arabidopsis is controlled by STEROL METHYLTRANSFERASE 2;1. The Plant Journal 25(6), 605-615. doi: 10.1046/j.1365-313x.2001.00994.x.
Schaller, H. (2003). The role of sterols in plant growth and development. Progress in Lipid Research 42(3), 163-175. doi: 10.1016/S0163-7827(02)00047-4.
Schaller, H., Bouvier-Nave, P., and Benveniste, P. (1998). Overexpression of an Arabidopsis cDNA encoding a sterol-C24(1)-methyltransferase in tobacco modifies the ratio of 24-methyl cholesterol to sitosterol and is associated with growth reduction. Plant Physiology 118(2), 461-469. doi: 10.1104/pp. 118.2.461.
Schrick, K., Bruno, M., Khosla, A., Cox, P. N., Marlatt, S. A., Roque, R. A., et al. (2014). Shared functions of plant and mammalian StAR-related lipid transfer (START) domains in modulating transcription factor activity. BMC Biology 12(1), 70. doi: 10.1186/s12915-014-0070-8.
Schrick, K., Cordova, C., Li, G., Murray, L., and Fujioka, S. (2011). A dynamic role for sterols in embryogenesis of Pisum sativum. Phytochemistry 72(6), 465-475. doi: 10.1016/j.phytochem.2011.01.009.
Schrick, K., Mayer, U., Horrichs, A., Kuhnt, C., Bellini, C., Dangl, J., et al. (2000). FACKEL is a sterol C-14 reductase required for organized cell division and expansion in Arabidopsis embryogenesis. Genes and Development 14(12), 1471-1484. doi: 10.1101/gad.14.12.1471.
Schrick, K., Mayer, U., Martin, G., Bellini, C., Kuhnt, C., Schmidt, J., et al. (2002). Interactions between sterol biosynthesis genes in embryonic development of Arabidopsis. The Plant Journal 31(1), 61-73. doi: 10.1046/j.1365-313X.2002.01333.x.
Schrick, K., Nguyen, D., Karlowski, W. M., and Mayer, K. F. (2004). START lipid/sterol-binding domains are amplified in plants and are predominantly associated with homeodomain transcription factors. Genome Biology 5(6), R41. doi: 10.1186/gb-2004-5-6-r41.
Sekula, J., Niziol, J., Rode, W., and Ruman, T. (2015). Silver nanostructures in laser desorption/ionization mass spectrometry and mass spectrometry imaging. The Analyst 14(18), 6195-6209. doi: 10.1039/c5an00943j.
Senthil-Kumar, M., Wang, K., and Mysore, K. S. (2013). AtCYP710A1 gene-mediated stigmasterol production plays a role in imparting temperature stress tolerance in Arabidopsis thaliana. Plant Signaling and Behavior 8(2), e23142. doi: 10.4161/psb.23142.
Sonawane, P. D., Pollier, J., Panda, S., Szymanski, J., Massalha, H., Yona, M., et al. (2016). Plant cholesterol biosynthetic pathway overlaps with phytosterol metabolism. Nature Plants 3(1), 16205. doi: 10.1038/nplants.2016.205.
Souter, M., Topping, J., Pullen, M., Friml, J., Palme, K., Hackett, R., et al. (2002). hydra Mutants of Arabidopsis are defective in sterol profiles and auxin and ethylene signaling. The Plant Cell 14(5), 1017-1031.
Suza, W. P., and Chappell, J. (2016). Spatial and temporal regulation of sterol biosynthesis in Nicotiana benthamiana. Physiologia Plantarum 157(2), 120-134. doi: 10.1111/pp1.12413.
Suza, W. P., and Staswick, P. E. (2008). The role of JAR1 in Jasmonoyl-L-isoleucine production during Arabidopsis wound response. Planta 227(6), 1221-1232. doi: 10.1007/s00425-008-0694-4.
Valitova, J. N., Sulkarnayeva, A. G., and Minibayeva, F. V. (2016). Plant sterols: Diversity, biosynthesis, and physiological functions. Biochemistry (Moscow)81(8), 819-834. doi: 10.1134/s0006297916080046.
Wang, K., Senthil-Kumar, M., Ryu, C.-M., Kang, L., and Mysore, K. S. (2012). Phytosterols play a key role in plant innate immunity against bacterial pathogens by regulating nutrient efflux into the apoplast. Plant Physiology 158(4), 1789-1802. doi: 10.1104/pp. 111.189217.
Whitaker, B. D., and Gapper, N. (2008). Ripening-specific stigmasterol increase in tomato fruit is associated with increased sterol C-22 desaturase (CYP710A11) gene expression. Journal of Agricultural and Food Chemistry 56(10), 3828-3835. doi: 10.1021/jf7037983.
Willemsen, V., Friml, J., Grebe, M., van den Toorn, A., Palme, K., and Scheres, B. (2003). Cell polarity and PIN protein positioning in Arabidopsis require STEROL METHYLTRANSFERASE1 function. The Plant Cell 15(3), 612-625.
Xia, J., Psychogios, N., Young, N., and Wishart, D. S. (2009). MetaboAnalyst: a web server for metabolomic data analysis and interpretation. Nucleic Acids Research 37 (Web Server), W652-W660. doi: 10.1093/nar/gkp356.
Xu, L., Kliman, M., Forsythe, J. G., Korade, Z., Hmelo, A. B., Porter, N. A., et al. (2015). Profiling and imaging ion mobility-mass spectrometry analysis of cholesterol and 7-dehydrocholesterol in cells via sputtered silver MALDI. Journal of the American Society for Mass Spectrometry 26(6), 924-933. doi: 10.1007/s13361-015-1131-0.
Yan, J., Lipka, A. E., Schmelz, E. A., Buckler, E. S., and Jander, G. (2015). Accumulation of 5-hydroxynorvaline in maize (Zea mays) leaves is induced by insect feeding and abiotic stress. Journal of Experimental Botany 66(2), 593-602. doi: 10.1093/jxb/eru385.
Yang, Q., Zhao, D., and Liu, Q. (2020). Connections between amino acid metabolisms in plants: Lysine as an example. Frontiers in Plant Science 11, 928. doi: 10.3389/fpls.2020.00928.

What is claimed is:

1. A modified plant, or a progeny, a plant part, or a plant cell thereof, with increased sitosterol or hard endosperm, the plant comprising reduced expression or activity of an endogenous CYP710A gene relative to a corresponding unmodified plant.

2. The modified plant of claim 1, wherein the endogenous CYP710A gene encodes a polypeptide comprising an amino acid sequence having at least 80%, at least 90%, at least 95%, at least 99%, or 100% sequence identity to SEQ ID NO: 2.

3. The modified plant of claim 1, wherein the endogenous CYP710A gene comprises a nucleotide sequence having at least 80%, at least 90%, at least 95%, at least 99%, or 100% sequence identity to SEQ ID NO: 1.

4. The modified plant of claim 1, wherein the modified plant comprises one or more nucleotide modifications at a genomic locus comprising the CYP710A gene.

5. The modified plant of claim 4, wherein the one or more nucleotide modifications are present within the coding region, non-coding region, regulatory sequence, or untranslated region of the endogenous CYP710A gene.

6. The modified plant of claim 4, wherein the one or more nucleotide modifications reduce expression of the CYP710A gene, reduce transcriptional activity of the polypeptide encoded by the CYP710A gene, generate one or more alternative spliced variants of the CYP710A gene, introduce a frameshift mutation in one or more exons of the CYP710A gene, delete a substantial portion of the CYP710A gene, delete a full-length open reading frame of the CYP710A gene, repress an enhancer motif present within a regulatory region encoding the CYP710A gene, or modify one or more nucleotides of a regulatory element operably linked to the CYP710A gene, or any combination thereof.

7. The modified plant of claim 1, wherein the modified plant comprises a silencing element that targets the endogenous CYP710A gene.

8. The modified plant of claim 7, wherein the silencing element comprises a double stranded RNA, a siRNA, a miRNA, or a hairpin suppression element.

9. The modified plant of claim 1, wherein the plant is a maize plant.

10. The modified plant of claim 1, wherein the plant comprises a nucleotide sequence having at least 80%, at least 90%, at least 95%, at least 99%, or 100% sequence identity to SEQ ID NO: 3.

11. The modified plant of claim 1, wherein the sitosterol is increased at least 1.2-fold, at least 1.3-fold, at least 1.4-fold, or at least 1.5-fold in the seed of the plant relative to a corresponding unmodified plant.

12. A seed or an asexual propagate of the modified plant of claim 1.

13. A method for producing a plant with increased sitosterol or hard endosperm, the method comprising:
reducing expression or activity of an endogenous CYP710A gene in the plant.

14. The method of claim 13, wherein the endogenous CYP710A gene encodes a polypeptide comprising an amino acid sequence having at least 80%, at least 90%, at least 95%, at least 99%, or 100% sequence identity to SEQ ID NO: 2.

15. The method of claim 13, wherein the endogenous CYP710A gene comprises a nucleotide sequence having at least 80%, at least 90%, at least 95%, at least 99%, or 100% sequence identity to SEQ ID NO: 1.

16. The method of claim 13, wherein the expression or activity is reduced by introducing one or more nucleotide modifications at a genomic locus comprising the CYP710A gene.

17. The method of claim 16, wherein the one or more nucleotide modifications are introduced through targeted DNA modification.

18. The method of claim 16, wherein the one or more nucleotide modifications are introduced through use of a guide RNA and an RNA-guided endonuclease.

19. The method of claim 16, wherein the one or more nucleotide modifications are introduced within the coding region, non-coding region, regulatory sequence, or untranslated region of the endogenous CYP710A gene.

20. The method of claim 16, wherein the one or more nucleotide modifications reduce expression of the CYP710A gene, reduce transcriptional activity of the polypeptide encoded by the CYP710A gene, generate one or more alternative spliced variants of the CYP710A gene, introduce a frameshift mutation in one or more exons of the CYP710A gene, delete a substantial portion of the CYP710A gene, delete a full-length open reading frame of the CYP710A gene, repress an enhancer motif present within a regulatory region encoding the CYP710A gene, or modify one or more nucleotides of a regulatory element operably linked to the CYP710A gene, or any combination thereof.

21. The method of claim 13, wherein the expression or activity is reduced by introducing a silencing element that targets the endogenous CYP710A gene.

22. The method of claim 21, wherein the silencing element comprises a double stranded RNA, a siRNA, a miRNA, or a hairpin suppression element.

23. The method of claim 13, wherein the plant is a maize plant.

24. The method of claim 13, wherein the sitosterol is increased at least 1.2-fold, at least 1.3-fold, at least 1.4-fold, or at least 1.5-fold in the seed of the plant relative to a corresponding unmodified plant.

25. A method of producing a plant with increased sitosterol or hard endosperm, the method comprising:
(a) crossing the plant of claim 1 with itself or another plant to produce seed; and
(b) growing a progeny plant from the seed to produce a plant with increased sitosterol or hard endosperm.

26. The method of claim 25, further comprising:
(c) crossing the progeny plant with itself or another plant; and
(d) repeating steps (b) and (c) for an additional 0-7 generations to produce a plant having increased sitosterol or hard endosperm.

27. A crop comprising a plurality of the plants of claim 1 planted together in an agricultural field.

28. A method of producing seeds having increased sitosterol or hard endosperm from a plant crop, the method comprising:
cultivating a plurality of the plants of claim 1 as a plant crop, and harvesting seeds from the plant crop, wherein the seeds have increased sitosterol or hard endosperm relative to a corresponding unmodified plant.

29. A commodity plant product prepared from the plant, plant part, or plant cell of claim 1, wherein the commodity plant product comprises a nucleic acid comprising one or more nucleotide modifications at a genomic locus comprising the CYP710A gene.

30. The commodity plant product of claim 29, wherein the product is fodder, seed meal, oil, or seed-treatment-coated seed.

31. A method for producing a commodity plant product, the method comprising processing the plant or plant part of claim 1 to obtain the product.

32. The method of claim 31, wherein the commodity plant product is fodder, seed meal, oil, or seed-treatment-coated seeds.


SEQUENCE LISTING (3 sequences)

SEQ ID NO: 1 - DNA, 1542 bp, Zea mays
  source 1..1542: mol_type = genomic DNA
atggcggcgcagtcactgtccgtgcccgtgcccgtgctcagctccttggacctccgcgcggcggcgccgttcctggttgccgcggtggccttgtacttcctgatggagcaggtgtcctaccaccggaagaaggggcccctgcccgggccggcgctcgtggtgccgttcctcggcagcgtggcgcacatgatccgcgacccgacggggttctgggacgcgcaggcggcgcgcgccaagcggtccggcgcgggcctcgccgccgacttcctggtggggcggttcgtggtgttcatccgcgactcggagctgtcccaccgcgtgttcgccaacgtccgccccgacgcgttccacctcatcggccaccccttcggcaagaagctgttcggcgaccacaacctcatctacatgttcggcgaggaccacaaggacctgcggcggcggatcgcgcccaacttcaccccgcgcgcgctgtccacctacgccgcgctccagcagcgcgtcatcctggcgcacctccggcggtggctcgaccggagcggcggcggggcgttccccatccgcgtgccctgccgcgacatgaacctggagacctcgcagacggtgttcgtggggccgtacctcgccggggaggcccggcggcggttcgagcgggactacaacctcttcaacgtcgggctcatggcgctgcccatcgacctgccctgcttcgcgttccggcgcgccagggagggcgtggcgcggctggtgcgcacgctgggcgagtgcgcgcggcagagcaaggcgcggatgcgcgccggcggcgagcccgagtgcctggtggacttctggatgcaggacacgctgcgggagatggacgaggcggcggcggcggggcgcccgccccccgcgcacaccgacgacgaggagatcggcgggttcatgttcgacttcctgttcgcggcgcaggacgcgtccacctcgtccctctgctgggcggtgtcggcgctggactcccacccggaggtgctggcccgcgtgcgcgccgaggtgtcggccgcctggtcgccggactccggcgagccgatgacggcggagacgatccaggggatgcggtacacgcaggcggtggcgcgcgaggtggtccggcaccggccccccgcgacgctggtgccgcacatcgcgggcgaggcgttccagctgacggagtggtacacggtgcccaagggcgccatcgtgttcccgtccgtgtacgagtcgtcgttccaggggttcccggacgcggaggcgttcgacccggagcgcttcttctcggaggcgcggcgggaggacgtggcgtacaggcgcaacttcctggcattcggcgccgggccgcaccagtgcgtggggcagcggtacgcgctgaaccacctcgtcctgttcatggcgctgttcgtgtcggtggtggacttccggcgggagaggacgcccgggtgtgatgacccggtgtacatgcccaccatcgtgcccaaggacggctgctccgtgtacctcaagcagcgatgcgccaagttgccgtcgttctga

SEQ ID NO: 2 - protein, 513 aa, Zea mays
MAAQSLSVPVPVLSSLDLRAAAPFLVAAVALYFLMEQVSYHRKKGPLPGPALVVPFLGSVAHMIRDPTGFWDAQAARAKRSGAGLAADFLVGRFVVFIRDSELSHRVFANVRPDAFHLIGHPFGKKLFGDHNLIYMFGEDHKDLRRRIAPNFTPRALSTYAALQQRVILAHLRRWLDRSGGGAFPIRVPCRDMNLETSQTVFVGPYLAGEARRRFERDYNLFNVGLMALPIDLPCFAFRRAREGVARLVRTLGECARQSKARMRAGGEPECLVDFWMQDTLREMDEAAAAGRPPPAHTDDEEIGGFMFDFLFAAQDASTSSLCWAVSALDSHPEVLARVRAEVSAAWSPDSGEPMTAETIQGMRYTQAVAREVVRHRPPATLVPHIAGEAFQLTEWYTVPKGAIVFPSVYESSFQGFPDAEAFDPERFFSEARREDVAYRRNFLAFGAGPHQCVGQRYALNHLVLFMALFVSVVDFRRERTPGCDDPVYMPTIVPKDGCSVYLKQRCAKLPSF

SEQ ID NO: 3 - DNA, 1680 bp, synthetic construct
  source 1..1680: mol_type = other DNA
  misc_feature 1436: note = n indicates an unknown length of sequence present within the transposon that was not determined
atggcggcgcagtcactgtccgtgcccgtgcccgtgctcagctccttggacctccgcgcggcggcgccgttcctggttgccgcggtggccttgtacttcctgatggagcaggtgtcctaccaccggaagaaggggcccctgcccgggccggcgctcgtggtgccgttcctcggcagcgtggcgcacatgatccgcgacccgacggggttctgggacgcgcaggcggcgcgcgccaagcggtccggcgcgggcctcgccgccgacttcctggtggggcggttcgtggtgttcatccgcgactcggagctgtcccaccgcgtgttcgccaacgtccgccccgacgcgttccacctcatcggccaccccttcggcaagaagctgttcggcgaccacaacctcatctacatgttcggcgaggaccacaaggacctgcggcggcggatcgcgcccaacttcaccccgcgcgcgctgtccacctacgccgcgctccagcagcgcgtcatcctggcgcacctccggcggtggctcgaccggagcggcggcggggcgttccccatccgcgtgccctgccgcgacatgaacctggagacctcgcagacggtgttcgtggggccgtacctcgccggggaggcccggcggcggttcgagcgggactacaacctcttcaacgtcgggctcatggcgctgcccatcgacctgccctgcttcgcgttccggcgcgccagggagggcgtggcgcggctggtgcgcacgctgggcgagtgcgcgcggcagagcaaggcgcggatgcgcgccggcggcgagcccgagtgcctggtggacttctggatgcaggacacgctgcgggagatggacgaggcggcggcggcggggcgcccgccccccgcgcacaccgacgacgaggagatcggcgggttcatgttcgacttcctgttcgcggcgcaggacgcgtccacctcgtccctctgctgggcggtgtcggcgctggactcccacccggaggtgctggcccgcgtgcgcgccgaggtgtcggccgcctggtcgccggactccggcgagccgatgacggcggagacgatccaggggatgcggtacacgcaggcggtggcgcgcgaggtggtccggcaccggccccccgcgacgctggtgccgcacatcgcgggcgaggcgttccagctgacggagtggtacacggtgcccaagggcgccatcgtgttcccgtccgtgtacgagtcgtcgttccaggggttcccggacgcggaggcgttcgacccggagcgcttcttctcggaggcgcggcgggaggacgtggcgtacaggcgcaacttcctggcattcggcgccgggccgcaccagtgcgtggggcagcggtacgcggagataattgtcattatagacgaagagcggacgggattcgacgaaatagaggcgatggngagaagccaacgccatcgcctccatttcgtcgaatcccgtccgctcttcgtctataatggcaattatctccggtacgcgctgaaccacctcgtcctgttcatggcgctgttcgtgtcggtggtggacttccggcgggagaggacgcccgggtgtgatgacccggtgtacatgcccaccatcgtgcccaaggacggctgctccgtgtacctcaagcagcgatgcgccaagttgccgtcgttctga